SEQUENCE LISTING (62 sequences)

SEQ ID NO: 1 - protein, 13 aa, Synthetic construct
  source 1..13: note = LCDR1
SGSSSNIGNHYVN

SEQ ID NO: 2 - protein, 7 aa, Synthetic construct
  source 1..7: note = LCDR2
RNNHRPS

SEQ ID NO: 3 - protein, 11 aa, Synthetic construct
  source 1..11: note = LCDR3
QSWDYSGFSTV

SEQ ID NO: 4 - protein, 13 aa, Synthetic construct
  source 1..13: note = LCDR1
SGSSSNIGNHYVN

SEQ ID NO: 5 - protein, 7 aa, Synthetic construct
  source 1..7: note = LCDR2
RNNHRPS

SEQ ID NO: 6 - protein, 11 aa, Synthetic construct
  source 1..11: note = LCDR3
QSWDYSGFSTV

SEQ ID NO: 7 - protein, 9 aa, Synthetic construct
  source 1..9: note = LCDR1
SSSNIGNHY

SEQ ID NO: 8 - skipped

SEQ ID NO: 9 - protein, 8 aa, Synthetic construct
  source 1..8: note = LCDR3
WDYSGFST

SEQ ID NO: 10 - protein, 8 aa, Synthetic construct
  source 1..8: note = LCDR1
SSNIGNHY

SEQ ID NO: 11 - skipped

SEQ ID NO: 12 - protein, 11 aa, Synthetic construct
  source 1..11: note = LCDR3
QSWDYSGFSTV

SEQ ID NO: 13 - protein, 110 aa, Synthetic construct
  source 1..110: note = VL
DIVLTQPPSVSGAPGQRVTISCSGSSSNIGNHYVNWYQQLPGTAPKLLIYRNNHRPSGVPDRFSGSKSGTSASLAITGLQSEDEADYYCQSWDYSGFSTVFGGGTKLTVL

SEQ ID NO: 14 - protein, 216 aa, Synthetic construct
  source 1..216: note = Light chain 1
DIVLTQPPSVSGAPGQRVTISCSGSSSNIGNHYVNWYQQLPGTAPKLLIYRNNHRPSGVPDRFSGSKSGTSASLAITGLQSEDEADYYCQSWDYSGFSTVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 15 - protein, 15 aa, Synthetic construct
  source 1..15: note = LCDR1
RASKSVDSYGNSFMH

SEQ ID NO: 16 - protein, 7 aa, Synthetic construct
  source 1..7: note = LCDR2
LASNLES

SEQ ID NO: 17 - protein, 9 aa, Synthetic construct
  source 1..9: note = LCDR3
QQNNEDPRT

SEQ ID NO: 18 - protein, 15 aa, Synthetic construct
  source 1..15: note = LCDR1
RASKSVDSYGNSFMH

SEQ ID NO: 19 - protein, 7 aa, Synthetic construct
  source 1..7: note = LCDR2
LASNLES

SEQ ID NO: 20 - protein, 9 aa, Synthetic construct
  source 1..9: note = LCDR3
QQNNEDPRT

SEQ ID NO: 21 - protein, 11 aa, Synthetic construct
  source 1..11: note = LCDR1
SKSVDSYGNSF

SEQ ID NO: 22 - skipped

SEQ ID NO: 23 - protein, 6 aa, Synthetic construct
  source 1..6: note = LCDR3
NNEDPR

SEQ ID NO: 24 - protein, 10 aa, Synthetic construct
  source 1..10: note = LCDR1
KSVDSYGNSF

SEQ ID NO: 25 - skipped

SEQ ID NO: 26 - protein, 9 aa, Synthetic construct
  source 1..9: note = LCDR3
QQNNEDPRT

SEQ ID NO: 27 - protein, 111 aa, Synthetic construct [sequence omitted]

SEQ ID NO: 28 - protein, 218 aa, Synthetic construct
  source 1..218: note = Light chain 2
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 10 aa, Synthetic construct
  source 1..10: note = HCDR1
GGTFKSYAIS

SEQ ID NO: 30 - protein, 17 aa, Synthetic construct
  source 1..17: note = HCDR2
NIIPMTGQTYYAQKFQG

SEQ ID NO: 31 - protein, 10 aa, Synthetic construct
  source 1..10: note = HCDR3
AAYHPLVFDN

SEQ ID NO: 32 - protein, 5 aa, Synthetic construct
  source 1..5: note = HCDR1
SYAIS

SEQ ID NO: 33 - protein, 17 aa, Synthetic construct
  source 1..17: note = HCDR2
NIIPMTGQTYYAQKFQG

SEQ ID NO: 34 - protein, 10 aa, Synthetic construct
  source 1..10: note = HCDR3
AAYHPLVFDN

SEQ ID NO: 35 - protein, 7 aa, Synthetic construct
  source 1..7: note = HCDR1
GGTFKSY

SEQ ID NO: 36 - protein, 6 aa, Synthetic construct
  source 1..6: note = HCDR2
IPMTGQ

SEQ ID NO: 37 - protein, 10 aa, Synthetic construct
  source 1..10: note = HCDR3
AAYHPLVFDN

SEQ ID NO: 38 - protein, 8 aa, Synthetic construct
  source 1..8: note = HCDR1
GGTFKSYA

SEQ ID NO: 39 - protein, 8 aa, Synthetic construct
  source 1..8: note = HCDR2
IIPMTGQT

SEQ ID NO: 40 - protein, 12 aa, Synthetic construct
  source 1..12: note = HCDR3
ARAAYHPLVFDN

SEQ ID NO: 41 - protein, 119 aa, Synthetic construct
  source 1..119: note = VH
EVQLVQSGAEVKKPGSSVKVSCKASGGTFKSYAISWVRQAPGQGLEWMGNIIPMTGQTYYAQKFQGRVTITADESTSTAYMELSSLRSEDTAVYYCARAAYHPLVFDNWGQGTLVTVSS

SEQ ID NO: 42 - protein, 449 aa, Synthetic construct
  source 1..449: note = Heavy Chain 1
EVQLVQSGAEVKKPGSSVKVSCKASGGTFKSYAISWVRQAPGQGLEWMGNIIPMTGQTYYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 10 aa, Synthetic construct
  source 1..10: note = HCDR1
GFSLSAYSVN

SEQ ID NO: 44 - protein, 16 aa, Synthetic construct
  source 1..16: note = HCDR2
MIWGDGKIVYNSALKS

SEQ ID NO: 45 - protein, 10 aa, Synthetic construct
  source 1..10: note = HCDR3
DGYYPYAMDN

SEQ ID NO: 46 - protein, 5 aa, Synthetic construct
  source 1..5: note = HCDR1
AYSVN

SEQ ID NO: 47 - protein, 16 aa, Synthetic construct
  source 1..16: note = HCDR2
MIWGDGKIVYNSALKS

SEQ ID NO: 48 - protein, 10 aa, Synthetic construct
  source 1..10: note = HCDR3
DGYYPYAMDN

SEQ ID NO: 49 - protein, 7 aa, Synthetic construct
  source 1..7: note = HCDR1
GFSLSAY

SEQ ID NO: 50 - protein, 5 aa, Synthetic construct
  source 1..5: note = HCDR2
WGDGK

SEQ ID NO: 51 - protein, 10 aa, Synthetic construct
  source 1..10: note = HCDR3
DGYYPYAMDN

SEQ ID NO: 52 - protein, 8 aa, Synthetic construct
  source 1..8: note = HCDR1
GFSLSAYS

SEQ ID NO: 53 - protein, 7 aa, Synthetic construct
  source 1..7: note = HCDR2
IWGDGKI

SEQ ID NO: 54 - protein, 12 aa, Synthetic construct
  source 1..12: note = HCDR3
AGDGYYPYAMDN

SEQ ID NO: 55 - protein, 118 aa, Synthetic construct [sequence omitted]

SEQ ID NO: 56 - protein, 448 aa, Synthetic construct
  source 1..448: note = Heavy Chain 2
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 449 aa, Synthetic construct
  source 1..449: note = Heavy chain 1
EVQLVQSGAEVKKPGSSVKVSCKASGGTFKSYAISWVRQAPGQGLEWMGNIIPMTGQTYYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 448 aa, Synthetic construct
  source 1..448: note = Heavy chain 2
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 59 - protein, 227 aa, Synthetic construct
  source 1..227: note = Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 227 aa, Synthetic construct
  source 1..227: note = Fc-hole
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 61 - protein, 227 aa, Synthetic construct
  source 1..227: note = Fc-key
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 193 aa, Synthetic construct
  source 1..193: note = IL-18
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

(19) United States
(12) Patent Application Publication
(10) Pub. No.: US 2026/0297177 A1
(43) Pub. Date: Oct. 1, 2026

(54) PHARMACEUTICAL COMPOSITIONS COMPRISING MULTISPECIFIC ANTIBODIES

(71) Applicant: NOVARTIS AG, Basel (CH)

(72) Inventors: Shwetha IYER, Watertown, MA (US); Artemiza Sorina MORAR-MITRICA, Basel (CH); Ankita PANDEY, Indore (IN); Dario SPIGOLON, Lugano (CH); Jacqueline ZUCKETTO, Würselen (DE)

(21) Appl. No.: 19/633,231

(22) Filed: Mar. 30, 2026

(30) Foreign Application Priority Data
Apr. 1, 2025 (IN) 202511032373

(51) Int. Cl. C07K 16/24; A61K 39/00; A61K 47/18; A61K 47/20; A61K 47/22; A61K 47/26

(52) U.S. Cl. CPC C07K 16/244; A61K 47/183; A61K 47/20; A61K 47/22; A61K 47/26; A61K 2039/54; A61K 2039/545; C07K 2317/31; C07K 2317/52; C07K 2317/565; C07K 2317/94

(57) ABSTRACT

The invention relates to pharmaceutical compositions comprising multispecific antibodies, pharmaceutical products comprising said pharmaceutical compositions, and methods of using said pharmaceutical compositions.

### FIELD

The disclosure relates to pharmaceutical compositions comprising multispecific antibodies, e.g., anti-IL-18/IL-13 multispecific antibodies, pharmaceutical products comprising the pharmaceutical compositions, and methods of using the pharmaceutical compositions.

### SEQUENCE LISTING

The instant application contains a Sequence Listing which has been submitted electronically in XML format and is hereby incorporated by reference in its entirety. The XML copy, created on Mar. 12, 2026, is named “PAT060029-PCT-SEC01_Sequence listing.xml) and is 106,496 bytes in size.

### INCORPORATION BY REFERENCE

This application claims priority to Indian Patent Application number 202511032373, filed on Apr. 1, 2025, the entire contents of which are incorporated herein by reference. In addition, all publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present disclosure are intended.

### BACKGROUND

Interleukin-13 (IL-13) is a cytokine associated with the pathogenesis of inflammatory disorders such as atopic dermatitis (AD). Serum levels of IL-13 have been shown to correlate with disease severity. IL-18 is another cytokine associated with pathogenesis of inflammatory disorders and is further considered to be involved in pathogenesis of atopic dermatitis by inducing super Th1 cells to produce and secrete IFNγ and IL-13 (Terada et al. (2006) PNAS 103:8816-8821). IL-18 is released by keratinocytes and inflammatory dendritic cells, and serum IL-18 levels in patients with atopic dermatitis has shown to be significantly correlated with skin scores of AD lesions (lkezawa et al. (2010) AAIR 2 (4): 235-246).

Typical treatments of atopic dermatitis include topical lotions and moisturizers, topical corticosteroid ointments, creams or injections. Most treatment options, however, offer only temporary, incomplete, symptom relief. Moreover, many patients with moderate-to-severe atopic dermatitis become resistant to treatment by topical corticosteroids or by calcineurin inhibitors. Recently, a number of biologics have become available for atopic dermatitis treatment. Dupilumab, available under the commercial name Ebglyss®, is a monoclonal anti-IL-4Rα antibody that is approved for the treatment of moderate-to-severe atopic dermatitis, with other biologics such as the anti-IL-13 monoclonal antibodies lebrikizumab (Ebglyss®) and tralokinumab (Adtralza®) also being available. WO2023/209568 describes multispecific, such as bispecific, antibodies binding to IL-18 and IL-13 that can be used in the treatment of atopic dermatitis.

Liquid pharmaceutical compositions formulated to contain high concentrations of an antibody often have short shelf lives and the formulated antibodies may lose biological activity resulting from chemical and physical instability during storage. In particular, aggregation, deamidation and oxidation are known to be the most common causes of antibody degradation. Further, aggregation can potentially lead to increased immune response in patients, leading to safety concerns. Thus, antibody aggregation in pharmaceutical compositions must be minimized or prevented. A commercial ready-to-use liquid antibody composition should provide sufficient physical and chemical stability of the antibody during shipping and handling to ensure that the dose and product safety claims are met when the molecule is administered to a patient. Moreover, the composition must also be of acceptable osmolality and pH value for subcutaneous application and have low viscosity as a prerequisite for manufacturing (compounding, filtration, filling) and syringeability, which can be challenging, particularly in the case of multispecific antibodies. Balancing these requirements is difficult, making the production of a commercially viable liquid pharmaceutical compositions a technical challenge.

Accordingly, there is still a need for improved pharmaceutical compositions comprising antibodies, particularly multispecific antibodies, that can be used in the treatment of inflammatory and/or immune conditions such as atopic dermatitis.

### SUMMARY

The present invention provides novel pharmaceutical compositions comprising a multispecific antibody or an antigen-binding fragment thereof. The pharmaceutical compositions described herein allow formulation of the multispecific antibody or antigen-binding fragment thereof at high concentrations while maintaining low viscosity and excellent properties, such as one or more properties selected from the group consisting of conformational (thermal) stability, physical stability, colloidal stability, chemical stability, and biological activity. The pharmaceutical compositions described herein are particularly suitable for the treatment of inflammatory and/or immune conditions. The pharmaceutical compositions described herein are suitable for subcutaneous administration. Thus, the pharmaceutical compositions described herein solve one or more of the current needs in the art.

Accordingly, in an aspect, there is provided a pharmaceutical composition comprising a multispecific antibody, wherein the multispecific antibody comprises:
i) a first part that binds to IL-18 comprising:
   an HCDR1 comprising SEQ ID NO: 32;
   an HCDR2 comprising SEQ ID NO: 33;
   an HCDR3 comprising SEQ ID NO: 34;
   an LCDR1 comprising SEQ ID NO: 4;
   an LCDR2 comprising SEQ ID NO: 5, and;
   an LCDR3 comprising SEQ ID NO: 6, and;
ii) a second part that binds to IL-13 comprising:
   an HCDR1 comprising SEQ ID NO: 46;
   an HCDR2 comprising SEQ ID NO: 47;
   an HCDR3 comprising SEQ ID NO: 48;
   an LCDR1 comprising SEQ ID NO: 18;
   an LCDR2 comprising SEQ ID NO: 19, and;
   an LCDR3 comprising SEQ ID NO: 20,
and wherein the pharmaceutical composition comprises from about 15 mg/mL to about 200 mg/mL of the multispecific antibody.

In preferred embodiments, the pharmaceutical composition is a liquid pharmaceutical composition, e.g., an aqueous pharmaceutical composition. In preferred embodiments, the multispecific antibody is bispecific.

In some embodiments, the pharmaceutical composition has a viscosity of about ≤20 cP, e.g., about ≤15 cP, measured at a temperature of about 25° C.

In some embodiments, the pharmaceutical composition comprises from about 100 mM to about 250 mM of a viscosity reducing agent. In some embodiments, the viscosity reducing agent is selected from the group consisting of lysine, arginine, glycinamide hydrochloride, MgCl₂, NaCl, and a combination thereof. In some embodiments, the pharmaceutical composition comprises arginine (e.g., L-arginine) or lysine (e.g., L-lysine), or a pharmaceutically acceptable salt thereof.

In some embodiments, the pharmaceutical composition comprises from about 100 mM to about 175 mM arginine (e.g., L-arginine) or lysine (e.g., L-lysine), or a pharmaceutically acceptable salt thereof. In some embodiments, the pharmaceutical composition comprises about 150 mM arginine (e.g., L-arginine) or lysine (e.g., L-lysine), preferably about 150 mM lysine (e.g., L-lysine). In some embodiments, the lysine is L-lysine or a pharmaceutically acceptable salt thereof, optionally L-lysine HCl.

In some embodiments, the pharmaceutical composition has a pH of from about 5 to about 8. In some embodiments, the pharmaceutical composition has a pH of from about 5.5 to about 7.4. In some embodiments, the pharmaceutical composition has a pH of from about 6 to about 7. In some embodiments, a preferred pharmaceutical composition has a pH of about 6.5.

In some embodiments, the pharmaceutical composition comprises from about 50 mg/mL to about 175 mg/mL, from about 75 mg/mL to about 175 mg/mL, or from about 100 mg/mL to about 175 mg/mL of the multispecific antibody. In some embodiments, a preferred pharmaceutical composition comprises about 150 mg/mL of the multispecific antibody.

In some embodiments, the pharmaceutical composition comprises methionine or a pharmaceutically acceptable salt thereof. In some embodiments, the pharmaceutical composition comprises from about 2.5 mM to about 20 mM methionine or a pharmaceutically acceptable salt thereof. In some embodiments, the pharmaceutical composition comprises about 5 mM methionine or a pharmaceutically acceptable salt thereof. In some embodiments, the methionine is L-methionine or a pharmaceutically acceptable salt thereof.

In some embodiments, the pharmaceutical composition comprises a sugar stabilizer. In some embodiments, the pharmaceutical composition comprises from about 50 mM to about 220 mM of the sugar stabilizer. In some embodiments, a preferred sugar stabilizer is trehalose or sucrose. In some embodiments, the pharmaceutical composition comprises from about 100 mM to about 150 mM trehalose or sucrose, e.g., about 120 mM trehalose or sucrose. In some embodiments, the pharmaceutical composition comprises about 120 mM trehalose. In some embodiments, the trehalose is trehalose dihydrate.

In some embodiments, the pharmaceutical composition comprises a buffering agent. In some embodiments, the pharmaceutical composition comprises from about 5 mM to about 50 mM of the buffering agent. In some embodiments, the buffering agent is selected from the group consisting of an acetate buffer, a citrate buffer, a phosphate buffer, a Tris buffer, a histidine buffer, and a combination thereof.

In some embodiments, a preferred pharmaceutical composition comprises from about 10 mM to about 25 mM of a histidine buffer, preferably about 20 mM of a L-histidine buffer. In some embodiments, the histidine buffer is an L-His/L-His HCl buffer. In some embodiments, the pharmaceutical composition comprises about 20 mM L-His/L-His HCl buffer.

In some embodiments, the pharmaceutical composition comprises a surfactant. In some embodiments, the pharmaceutical composition comprises from about 0.01% to about 0.1% (w/v) of the surfactant. In some embodiments, the surfactant is selected from the group consisting of polysorbate 80 (PS80), polysorbate 20 (PS20), Polox188, and a combination thereof. In some embodiments, a preferred pharmaceutical composition comprises from about 0.02% to 0.08% (w/v) polysorbate 80 (PS80) or polysorbate 20 (PS20). In some embodiments, the pharmaceutical composition comprises about 0.04% (w/v) polysorbate 80 (PS80).

In some embodiments, the first part that binds to IL-18 comprises a first variable heavy region (VH1) comprising SEQ ID NO: 41 and a first variable light region (VL1) comprising SEQ ID NO: 13, and the second part that binds to IL-13 comprises a second variable heavy region (VH2) comprising SEQ ID NO: 55 and a second variable light region (VL2) comprising SEQ ID NO: 27. In some embodiments, the first part that binds to IL-18 comprises a first light chain (LC1) that is a lambda light chain and the second part that binds to IL-13 comprises a second light chain (LC2) that is a kappa light chain. In some embodiments, a preferred first part that binds to IL-18 comprises a first light chain (LC1) comprising SEQ ID NO: 14 and a preferred second part that binds to IL-13 comprises a second light chain (LC2) comprising SEQ ID NO: 28.

In some embodiments, the first part that binds to IL-18 comprises a first Fc polypeptide, and the second part that binds to IL-13 comprises a second Fc polypeptide, and the first and second Fc polypeptides are associated forming an Fc region. In some embodiments, the first and/or second Fc polypeptide comprises the amino acid sequence SEQ ID NO: 59 or a variant thereof.

In some embodiments, the first and/or second Fc polypeptide comprises a variant of SEQ ID NO: 59 comprising one or more amino acid substitutions selected from the group consisting of:
a. one or more half-life extension substitutions, e.g., an M252Y, an S254T, and a T256E substitution (“YTE”), and;
b one or more K-i-H substitutions, e.g., an S354C and/or a T366W substitution or a Y349C, a T366S, an L368A, and/or a Y407V substitution, according to EU numbering.

In preferred embodiments,
the first part that binds to IL-18 comprises a first Fc polypeptide comprising a variant of SEQ ID NO: 59 comprising:
a) an M252Y, an S254T, and a T256E substitution (“YTE”), and;
b) a Y349C, a T366S, an L368A, and a Y407V substitution, and;
   the second part that binds to IL-13 comprises a second Fc polypeptide comprising a variant of SEQ ID NO: 59 comprising:
c) an M252Y, an S254T, and a T256E substitution (“YTE”), and;
d) an S354C and a T366W substitution, or
   the first part that binds to IL-18 comprises a first Fc polypeptide comprising a variant of SEQ ID NO: 59 comprising:
a) an M252Y, an S254T, and a T256E substitution (“YTE”), and;
b) an S354C and a T366W substitution, and;
   the second part that binds to IL-13 comprises a second Fc polypeptide comprising a variant of SEQ ID NO: 59 comprising:
c) an M252Y, an S254T, and a T256E substitution (“YTE”), and;
d) a Y349C, a T366S, an L368A, and a Y407V substitution,
according to EU numbering.

In some embodiments, the multispecific antibody is bispecific and comprises a first part that binds to IL-18 comprising a first heavy chain (HCl) comprising SEQ ID NO: 42 and a first light chain (LC1) comprising SEQ ID NO: 14, and a second part that binds to IL-13 comprising a second heavy chain (HC2) comprising SEQ ID NO: 56 and a second light chain (LC2) comprising SEQ ID NO: 28.

In some embodiments, the multispecific antibody is bispecific and comprises a first part that binds to IL-18 comprising a first heavy chain (HCl) comprising SEQ ID NO: 57 and a first light chain (LC1) comprising SEQ ID NO: 14, and a second part that binds to IL-13 comprising a second heavy chain (HC2) comprising SEQ ID NO: 58 and a second light chain (LC2) comprising SEQ ID NO: 28.

In preferred embodiments, the IL-18 and IL-13 are human.

In some embodiments, the pharmaceutical composition is a stable pharmaceutical composition, preferably a stable liquid pharmaceutical composition. In some embodiments, the pharmaceutical composition is stable at a temperature of about 5° C. for at least 6 months. In some embodiments, the pharmaceutical composition is stable at a temperature of about 25° C. for at least 3 months. In some embodiments, the pharmaceutical composition is stable at a temperature of about 40° C. for at least 4 weeks.

In some embodiments, the pharmaceutical composition is suitable for subcutaneous administration.

In some embodiments, the pharmaceutical composition has a pH of from about 5 to about 8, and the multispecific antibody is bispecific and comprises:
i) a first part that binds to IL-18 comprising a first heavy chain (HCl) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28, and the pharmaceutical composition is a liquid pharmaceutical composition comprising:
   from about 100 mg/mL to about 175 mg/mL of the multispecific antibody;
   from about 100 mM to about 175 mM lysine (e.g., L-lysine) or a pharmaceutically acceptable salt thereof (e.g., L-lysine HCl).

In some embodiments, the pharmaceutical composition has a pH of from about 5 to about 8, and the multispecific antibody is bispecific and comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28, and the pharmaceutical composition is a liquid pharmaceutical composition comprising:
   from about 100 mg/mL to about 175 mg/mL of the multispecific antibody;
   from about 100 mM to about 175 mM lysine (e.g., L-lysine) or a pharmaceutically acceptable salt thereof (e.g., L-lysine HCl).

In some embodiments, the pharmaceutical composition comprises about 150 mg/mL of the bispecific antibody. In some embodiments, the pharmaceutical composition comprises from about 2.5 mM to about 20 mM methionine (e.g., L-methionine) or a pharmaceutically acceptable salt thereof. In some embodiments, the pharmaceutical composition comprises about 5 mM methionine (e.g., L-methionine) or a pharmaceutically acceptable salt thereof. In some embodiments, the pharmaceutical composition comprises from about 100 mM to about 150 mM trehalose (e.g., trehalose dihydrate). In some embodiments, the pharmaceutical composition comprises about 120 mM trehalose (e.g., trehalose dihydrate). In some embodiments, the pharmaceutical composition comprises from about 10 mM to about 25 mM of a histidine buffer (e.g., L-His/L-His HCl buffer). In some embodiments, the pharmaceutical composition comprises about 20 mM of a histidine buffer (e.g., L-His/L-His HCl buffer). In some embodiments, the pharmaceutical composition comprises from about 0.02% to about 0.08% (w/v) polysorbate 80 (PS80). In some embodiments, the pharmaceutical composition comprises about 0.04% (w/v) polysorbate 80. In some embodiments, the pharmaceutical composition has a pH of about 6.5.

In some embodiments, the pharmaceutical composition comprises:
about 150 mg/mL of a bispecific antibody comprising:
i) a first part that binds to IL-18 comprising a first heavy chain (HCl) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28;
   about 20 mM of a histidine buffer, e.g., L-His/L-His HCl buffer;
   about 120 mM trehalose, e.g., trehalose dihydrate;
   about 150 mM lysine (e.g., L-lysine) or a pharmaceutically acceptable salt thereof (e.g., L-lysine HCl);
   about 5 mM methionine (e.g., L-methionine) or a pharmaceutically acceptable salt thereof;
   about 0.04% (w/v) polysorbate 80 (PS80), and has a pH of about 6.5.

In some embodiments, the pharmaceutical composition comprises:
about 150 mg/mL of a bispecific antibody comprising:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) SEQ ID NO: 58 and a second light chain (LC2) comprising SEQ ID NO: 28;
   about 20 mM of a histidine buffer, e.g., L-His/L-His HCl buffer;
   about 120 mM trehalose, e.g., trehalose dihydrate;
   about 150 mM lysine (e.g., L-lysine) or a pharmaceutically acceptable salt thereof (e.g., L-lysine HCl);
   about 5 mM methionine (e.g., L-methionine) or a pharmaceutically acceptable salt thereof;
   about 0.04% (w/v) polysorbate 80 (PS80),
and has a pH of about 6.5.

In a further aspect, there is provided a pharmaceutical product comprising a container comprising a pharmaceutical composition described herein, optionally wherein the container is selected from the group consisting of a vial, an injection device, an injection pen, a vial and syringe, a cartridge, a pre-filled syringe, and an autoinjector. In some embodiments, the container comprises from about 1 mL to about 2 mL, e.g., about 1 mL or about 2 mL, injectable volume of the pharmaceutical composition.

In a further aspect, there is provided a kit comprising a pharmaceutical composition or a pharmaceutical product described herein, optionally further comprising instructions for use.

In a further aspect, there is provided a lyophilisate obtained by or obtainable by lyophilisation of a pharmaceutical composition described herein.

In a further aspect, there is provided a method of preventing and/or treating an IL-18 and/or IL-13 related disease or disorder in a subject in need thereof, comprising administering a therapeutically effective amount of a pharmaceutical composition described herein to the subject. In a further aspect, there is provided a pharmaceutical composition described herein for use in preventing and/or treating an IL-18 and/or IL-13 related disease or disorder in a subject in need thereof. In a further aspect, there is provided a use of a pharmaceutical composition described herein for the manufacture of a medicament for preventing and/or treating an IL-18 and/or IL-13 related disease or disorder in a subject in need thereof.

In some embodiments, the IL-18 and/or IL-13 related disease or disorder is an inflammatory and/or immune disease or disorder. In some embodiments, the IL-18 and/or IL-13 related disease or disorder is a skin disease or disorder. In some embodiments, the IL-18 and/or IL-13 related disease or disorder is atopic dermatitis or a related condition. In some embodiments, the atopic dermatitis is moderate-to-severe atopic dermatitis. In some embodiments, a preferred subject is a human.

### BRIEF DESCRIPTION OF DRAWINGS

FIG. 1 shows measured anti-IL-18/IL-13 bispecific antibody kD (mL/g), Tₒₙₛₑₜ and Tₘ (° C.) values in tested formulations of Example 1, with different pH values, buffer types and concentrations, and presence or absence of trehalose and lysine. Parameters were measured using an UNCLE instrument (Unchained Labs, CA, USA). Excipient concentrations shown in mM.

FIG. 2 shows measured anti-IL-18/IL-13 bispecific antibody Tₒₙₛₑₜ and Tₘ (° C.) in tested formulations of Example 2 vs theoretical pH and comprised sugar, as well as presence or absence of NaCl. FIG. 3 shows measured anti-IL-18/IL-13 bispecific antibody Tₒₙₛₑₜ and Tₘ (° C.) vs tested formulations. FIG. 4 shows measured anti-IL-18/IL-13 bispecific antibody Tₒₙₛₑₜ and Tₘ (° C.) vs buffer type and pH values. Parameters were measured using an UNCLE instrument (Unchained Labs, CA, USA).

FIG. 5 shows measured viscosity (cP) of tested formulations of Example 2 vs. pH, sugar, buffer type, and presence or absence of NaCl. FIG. 6 shows measured viscosity (cP) vs. theoretical pH, amino acid, buffer type, and presence or absence of NaCl. Viscosity measurements were performed using an Anton-Paar rheometer (Anton-Paar, Graz, AT), at 25° C. and a shear rate of 1000 s⁻¹.

FIG. 7 shows change of anti-IL-18/IL-13 bispecific antibody melting temperature in each formulation (ΔTₒₙₛₑₜ; ΔTₘ (C)) in formulations of Example 3 (histidine buffer, pH 5). Parameters were measured using an UNCLE instrument (Unchained Labs, CA, USA). Change in melting temperature is shown vs. minimal formulation (MF, 3-F1, buffer only).

FIG. 8 shows change in viscosity (cP) of tested formulations of Example 3 (histidine buffer, pH 5). Viscosity was measured using an UNCLE instrument (Unchained Labs, CA, USA). Change in viscosity is shown vs. minimal formulation (MF, 3-F1, buffer only).

FIG. 9 shows measured anti-IL-18/IL-13 bispecific antibody kD (mL/g) values, and FIG. 10 and FIG. 11 show measured anti-IL-18/IL-13 bispecific antibody mean Tₒₙₛₑₜ (° C.) and mean Tₘ (Tₘ1; Tₘ2; (° C.)) values, in tested formulations of Example 4 (pH 5.5-7). Parameters were measured using an UNCLE instrument (Unchained Labs, CA, USA).

FIG. 12 shows measured formulation osmolality values (mOsm/kg) of tested formulations of Example 5. Osmolality was measured using freezing point depression (FPD) in an Advanced Instruments Model 2020 osmometer (Advanced Instruments, MA, USA). MF: minimal formulation (buffer only); FF: full formulation.

FIG. 13 shows measured formulation viscosity values (cP) of tested formulations of Example 5. Viscosity measurements were performed using an Anton-Paar rheometer (Anton-Paar, Graz, AT), at 25° C. and a shear rate of 1000 s⁻¹. MF: minimal formulation (buffer only); FF: full formulation.

FIG. 14 shows measured anti-IL-18/IL-13 bispecific antibody Tₒₙₛₑₜ (° C.) and Tₘ; ° C.) values in tested formulations of Example 5. Parameters were measured using an UNCLE instrument (Unchained Labs, CA, USA). MF: minimal formulation (buffer only); FF: full formulation.

FIG. 15 shows measured turbidity (NTU) values of tested formulations of Example 5. For each formulation (left to right): TO, T2W25C (2 weeks at 25° C.), T4W25C (4 weeks at 25° C.), T3M25C (3 months at 25° C.), T2W40C (2 weeks at 40° C.), T4W40C (4 weeks at 40° C.), T3M5C (3 months at 5° C.), T6M5C (6 months at 5° C.). MF: minimal formulation (buffer only); FF: full formulation.

FIG. 16 shows measured change from TO in main purity peak (%) in tested formulations of Example 5, measured with SEC. For each formulation (left to right): Shaking stress, F/T (freeze-thaw), T2W25C (2 weeks at 25° C.), T4W25C (4 weeks at 25° C.), T3M25C (3 months at 25° C.), T2W40C (2 weeks at 40° C.), T4W40C (4 weeks at 40° C.), T3M5C (3 months at 5° C.), T6M5C (6 months at 5° C.). MF: minimal formulation (buffer only); FF: full formulation.

FIG. 17 shows measured change from TO in sum of fragments (%) in tested formulations of Example 5, measured with SEC. For each formulation (left to right): Shaking stress, F/T (freeze-thaw), T2W25C (2 weeks at 25° C.), T4W25C (4 weeks at 25° C.), T3M25C (3 months at 25° C.), T2W40C (2 weeks at 40° C.), T4W40C (4 weeks at 40° C.), T3M5C (3 months at 5° C.), T6M5C (6 months at 5° C.). MF: minimal formulation (buffer only); FF: full formulation.

FIG. 18 shows measured change from TO in sum of aggregates (%) in tested formulations of Example 5, measured with SEC. For each formulation (left to right): Shaking stress, F/T (freeze-thaw). MF: minimal formulation (buffer only); FF: full formulation.

FIG. 19 shows measured change from TO in basic variants (%) in tested formulations of Example 5, measured with CZE. For each formulation (left to right): T2W25C (2 weeks at 25° C.), T4W25C (4 weeks at 25° C.), T3M25C (3 months at 25° C.), T2W40C (2 weeks at 40° C.), T3M5C (3 months at 5° C.), T6M5C (6 months at 5° C.). MF: minimal formulation (buffer only); FF: full formulation.

FIG. 20 shows measured change from TO in acidic variants (%) in tested formulations of Example 5, measured with CZE. For each formulation (left to right): T2W25C (2 weeks at 25° C.), T4W25C (4 weeks at 25° C.), T3M25C (3 months at 25° C.), T2W40C (2 weeks at 40° C.), T4W40C (4 weeks at 40° C.), T3M5C (3 months at 5° C.), T6M5C (6 months at 5° C.). MF: minimal formulation (buffer only); FF: full formulation.

FIG. 21 shows relative change in deamidation (%) in the anti-IL-18/IL-13 bispecific antibody from TO (average) in tested formulations of Example 5 after 4 weeks storage at 40° C., measured with MAM-MS. Deamidation was monitored in amino acid residues N386, N391 of SEQ ID NO: 42; N385, N390 of SEQ ID NO: 56; N28, N31 of SEQ ID NO: 14.

FIG. 22 shows relative change in isomerization and succinimide formation (%) in the anti-IL-18/IL-13 bispecific antibody from TO (average) in tested formulations of Example 5 after 4 weeks storage at 40° C., measured with MAM-MS. Isomerization and succinimide formation was monitored in amino acid residues D54, D86, D89, D98, D106 of SEQ ID NO: 56.

FIG. 23 shows relative change in pyroglutamate formation (%) in the anti-IL-18/IL-13 bispecific antibody from TO (average) in tested formulations of Example 5 after 4 weeks storage at 40° C., measured with MAM-MS. Pyroglutamate formation was monitored in the N-terminal glutamic acid (E) residue of SEQ ID NO: 42.

FIG. 24 shows relative change anti-IL-18/IL-13 bispecific antibody oxidation from TO (average) in tested formulations of Example 5 after application of forced oxidation with H2O2. Oxidation was monitored in amino acid residue M37 of SEQ ID NO: 28.

FIG. 25 shows relative change in anti-IL-18/IL-13 bispecific antibody potency from TO in formulations of Example 5 comprising lysine, arginine, or high trehalose. MF: minimal formulation (buffer only); FF: full formulation.

FIG. 26 shows aggregation rate (s⁻¹) of anti-IL-18/IL-13 bispecific antibody under shear stress in formulations of Example 5 comprising lysine or arginine. Aggregation rate (AR) was measured utilizing an ARGEN (Aggregate Rate GENerator) instrument (Yokogawa Fluence Analytics, TX, USA), by applying 5 different stirring speed rpm values (250, 500, 750, 1000, and 1250 rpm) at 25° C. MF: minimal formulation (buffer only); FF: full formulation.

### DETAILED DESCRIPTION

Unless defined otherwise, all technical and scientific terms used herein have the same meaning as commonly understood by those of ordinary skill in the art to which this disclosure pertains.

The term “IL-18”, “IL18”, “IL-18 polypeptide”, or “IL-18 protein”, refers to interleukin-18, which is a pro-inflammatory cytokine associated with the induction of Th1 responses, enhanced type I macrophage activation and NK/CD8+ T-cell cytotoxicity (Okamura et al. (1995) Nature; 378:88-91; Yoshimoto et al. (1998) J Immunol; 161 (7): 3400-7; Arend et al. (2008) Immunol Rev.; 223:20-38). IL-18 was originally described in 1989 as interferon-gamma (IFNγ) inducing factor (IGIF). IL-18 is related to the IL-1 family and is structurally related to IL-1β (Okamura et al. (1995) Nature; 378:88-91). IL-18 is primarily produced by macrophages and T-cells as a precursor protein (pro-IL-18) and secreted as an active protein following cleavage by caspase-1 (Dinarello C A et al (1999) J Allergy Clin Immunol; 103:11-24). In addition to macrophages and T-cells, pro-IL-18 is produced by a wide variety of other cells, including keratinocytes, intestinal epithelial cells, and osteoblasts. Throughout this specification, the term IL-18 encompasses both pro-IL-18 (precursor of mature IL-18 prior to protease cleavage) and mature IL-18 (post protease cleavage) interchangeably unless it is specified that the pro- or mature form is meant. The term “IL-18” refers in particular to human IL-18, e.g., to human IL-18 comprising amino acids 37 to 193 of SEQ ID NO: 62.

IL-18 binds with high affinity and signals through the IL-18 receptor (IL-18R), a heteromeric complex of alpha and beta chains encoded by the genes IL18R1 and IL18RAP, respectively (Torigoe K et al (1997) J Biol Chem; 272 (41): 25737-42). The bioactivity of IL-18 is negatively regulated by the IL-18 binding protein (IL-18BP), a naturally occurring and highly specific inhibitor. This soluble protein forms a complex with free IL-18 preventing its interaction with the IL-18 receptor, thus neutralizing and inhibiting its biological activity (Dinarello CA (2000) Ann Rheum Dis; 59 Suppl 1:117-20). IL-18BP is a constitutively secreted protein with high affinity binding to IL-18. Alternate mRNA splicing variants of IL-18BP result in four isoforms. The prominent ‘a’ isoform is present in the serum of healthy humans at 20-fold molar excess compared with IL-18 (Dinarello and Kaplanski (2005) Expert Rev Clin Immunol, 1 (4), 619-632).

The term “IL-13”, “IL13” “IL-13 polypeptide”, or “IL-13 protein”, refers to interleukin 13, a cytokine involved in regulating inflammatory and immune responses. The human and murine amino acid and nucleic acid sequences can be found in public databases, such as GenBank, UniProt and Swiss-Prot. For example, the amino acid sequence of human IL-13 can be found under UniProt Accession No. P35225. The term “IL-13” refers in particular to human IL-13.

The anti-IL-18/IL-13 multispecific, e.g., bispecific, antibodies described herein and their antigen-binding fragments preferably specifically bind to IL-18 and IL-13, more preferably to human IL-18 and human IL-13.

As used herein, the singular forms “a,” “an” and “the” include plural references unless the content clearly dictates otherwise. Thus, for example, reference to “an antibody” includes a mixture of two or more such antibodies. Thus, unless stated otherwise, the terms are to be understood as “at least one”.

Unless otherwise specifically stated or clear from context, as used herein, the term “about” in relation to a numerical value is understood as being within the normal tolerance in the art, e.g., within two standard deviations of the mean. Thus, “about” can e.g., be within +/−20%, 15%, 10%, 9%, 8%, 7%, 6%, 5%, 4%, 3%, 2%, 1%, 0.1%, 0.05%, or 0.01% of the stated value, e.g., +/−20% or +/−10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The term “comprise”, “comprises”, “comprising”, and the like, encompasses “including” as well as “consisting of”, e.g., a composition “comprising” X may consist exclusively of X or may include something additional, e.g., X+Y. Similarly, a polypeptide or nucleic acid molecule “comprising” an amino acid sequence or a nucleotide sequence respectively, such as the ones described herein, may consist exclusively of the amino acid sequence or nucleotide sequence, or may include additional amino acid or nucleotide sequences. As used herein, the phrase “consisting essentially of” refers to the genera or species of active pharmaceutical agents included in a method or composition, as well as any excipients inactive for the intended purpose of the method or composition. The phrase “consisting essentially of” does not exclude the presence of additional excipients that do not materially affect the basic and novel characteristics of a pharmaceutical composition. In some cases, the phrase “consisting essentially of” expressly excludes the inclusion of one or more additional active agents other than an antibody of the present disclosure. In some cases, the phrase “consisting essentially of” expressly excludes the inclusion of one or more additional active agents other than an antibody of the present disclosure and a second co-administered active agent.

The term “binding specificity” as used herein refers to the ability of an individual antibody binding site to react with the greatest affinity to the specified antigenic determinant; e.g., an antibody may bind other antigens besides the specified antigen at a lower affinity (e.g., at a 10×, 100×, 1000× lower affinity).

The term “specifically binds” or “selectively binds”, when used in the context of describing the interaction of an antigen and a multispecific antibody or antigen-binding fragment thereof described herein, refers to a binding reaction that is determinative of the presence of a cognate antigen (e.g., IL-18 and/or IL-13) in a heterogeneous population of proteins and other biologics, e.g., in a biological sample (such as a blood, serum, cell, cell lysate, cell supernatant, plasma or tissue sample). Binding may also be called specific when the antibody or antigen-binding fragment thereof binds to its target(s) with a binding affinity that is at least two-fold, at least five-fold, or at least ten-fold greater than its affinity for binding to an antigen other than its target(s), or against the background in an immunoassay. Binding affinity can be measured according to standard methods in the art, for example with surface plasmon resonance using commercially available instruments and protocols (e.g., using a Biacore instrument (Cytiva Life Sciences, Uppsala, Sweden)) or solution equilibrium titration (SET). Other examples of suitable methods include enzyme-linked immunosorbent assay (ELISA), radioimmunoassay (REA), FACS analysis, bioassays (e.g., growth inhibition), and Western Blotting.

The term “KD” or “K_{D}” in the context of binding affinity refers to the dissociation equilibrium constant of a particular antibody-antigen interaction and provides a measurement of an antibody's affinity to its target. Comparison of affinity and/or binding properties between two antibodies can also be established without actually determining the KD value for each antibody, but based on a quantitative measurement of binding (e.g. by ELISA or FACS analysis) that is proportional to KD or on a qualitative measurement of affinity or on an inference of affinity (e.g., in functional assay or in vitro or in vivo assay with respect to the antibody exerting an effect associated with binding to its target).

In some embodiments, “specifically binds to IL-18” is intended to refer to a multispecific (e.g., bispecific) antibody or antigen-binding fragment thereof that binds to IL-18 with a KD of 100 nM or less, 10 nM or less, 1 nM or less, 100 μM or less, for example 10 μM or less, in particular as measured by SET. In some embodiments, “specifically binds to IL-13” is intended to refer to a multispecific (e.g., bispecific) antibody or antigen-binding fragment thereof that binds to IL-13 with a KD of 100 nM or less, 10 nM or less, 1 nM or less, 100 μM or less, for example 10 μM or less, in particular as measured by SET.

Preferably, the multispecific (e.g., bispecific) antibodies and antigen-binding fragments thereof described herein do not bind to the IL-18/IL-18 binding protein (IL-18BP) complex, i.e., the multispecific antibodies and antigen-binding fragments thereof preferably bind to “free” IL-18. In some embodiments, “does not bind to the IL-18/IL-18BP” complex refers to the antibody or antigen-binding fragment thereof binding to the IL-18/IL-18BP complex with a KD of 1×10⁻⁵ M or greater.

The term “specifically binds” does not exclude cross-species reactivity. For example, an antibody that “specifically binds” to an antigen from one species may also “specifically bind” to that antigen in one or more other species. Thus, such cross-species reactivity does not itself alter the classification of an antibody as a “specific” binder. In certain embodiments, an antibody that specifically binds to human IL-18 and human IL-13 has cross-species reactivity with IL-18 and/or IL-13 from one or more non-human mammalian species, e.g., a primate species (including but not limited to one or more of Macaca fascicularis, Macaca mulatta, and Macaca nemestrina) or a rodent species, e.g., Mus musculus.

The term “antibody” as used herein refers to a polypeptide (or set of polypeptides) of the immunoglobulin family that is capable of binding an antigen non-covalently, reversibly, and typically, specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The variable regions of both the light (VL) and heavy (VH) chain portions determine antigen recognition and specificity. Conversely, the constant regions of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus of the molecule contains the variable region, and the C-terminus contains the constant region; the CH3 and CL domains comprise the carboxy-terminus of the heavy and light chain, respectively. The constant regions typically have a more conserved amino acid sequence relative to the other portion of the immunoglobulin.

The term “antibody” includes, but is not limited to, monoclonal antibodies, human antibodies, humanized antibodies, camelid antibodies, chimeric antibodies, multispecific, e.g., bispecific antibodies and anti-idiotypic (anti-Id) antibodies (including, e.g., anti-Id antibodies to antibodies described herein), as well as parts, such as antigen-binding fragments, thereof. The antibodies can be of any isotype/class (e.g., IgG, IgE, IgM, IgD, IgA, or IgY) or subclass (e.g., IgG1, IgG2, IgG3, IgG4, IgA1, or IgA2). Preferred antibodies are of the IgG1 class. The term “antibody” also encompasses antibody fragments and antibody-like molecules i.e., molecules comprising an antibody or an antigen-binding fragment thereof e.g., a molecule comprising a first half antibody having a typical antibody structure (comprising two polypeptides, the first comprising a light chain, and the second comprising a heavy chain) and a second half antibody having e.g., an scFv-Fc format.

The term “antibody fragment” as used herein refers to one or more portions of an antibody. In some embodiments, these portions are part of the constant domain(s) of an antibody, e.g., fragment crystallizable (Fc), constant (C) domains, etc. In some cases, these portions are antigen-binding fragments that retain the ability of binding an antigen. Thus, the term “antigen-binding fragment” as used herein refers to one or more portions of an antibody that retain the ability to bind to IL-18 and/or IL-13 (e.g., to IL-18 and IL-13), preferably specifically. Examples of antigen-binding fragments include, but are not limited to, a Fab fragment (comprising the VL, VH, CL and CH1 domains); a F (ab) 2 fragment, a bivalent fragment comprising two Fab fragments linked by a disulfide bridge at the hinge region; an Fd fragment comprising the VH and CH1 domains; an Fv fragment comprising the VL and VH domains of a single arm of an antibody, a single chain Fv fragment (scFv); an (scFv′) 2 (e.g., produced from scFv containing an additional C terminal cysteine by chemical coupling (Adams et al (1993) Can Res 53:4026-4034 and McCartney et al (1995) Protein Eng, 8:301-314) or by spontaneous site-specific dimerization of scFv containing an unpaired C terminal cysteine residue (Kipriyanov et al (1995) Cell. Bio Phys 26:187-204)); a single domain antibody (dAb) fragment (Ward et al., 1989 Nature 341:544-546), that comprises a VH domain or a VL domain; an isolated complementarity determining region (CDR), or other epitope-binding fragments of an antibody. ScFv-sc-Fv tandems ((scFv) 2) may also be produced by linking two scFv units by a third peptide linker, (see Kurucz et al (1995) J Immunol, 154:4576-4582).

Antigen-binding antibody fragments can be incorporated into e.g., single variable domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be incorporated into single chain molecules comprising a pair of tandem Fv segments (for example, VH-CH1-VH-CH1) which, together with complementary light chain polypeptides (for example, VL-VC-VL-VC), form a pair of antigen binding regions (Zapata et al., (1995) Protein Eng. 8:1057-1062; and U.S. Pat. No. 5,641,870). A single variable domain antibody may e.g. comprise a VH, VL, or VHH (variable region of heavy-chain antibody, e.g., a camelid or shark antibody), and may be present in a format (e.g., homo- or hetero-multimer) with other, different variable regions or variable domains where the other regions or domains are not required for antigen binding by the single chain variable domain.

As used herein, an “Fd” or “Fd region” comprises a VH and a CH1, and, optionally, any portion of an antibody hinge region.

The term “diabody” as used herein refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404097; WO 93/11161; and Hollinger et al., (1993) Proc. Natl. Acad. Sci. USA 90:6444-6448.

The term “single chain antibody”, “single chain Fv” or “scFv” refers to a molecule comprising an antibody heavy chain variable domain (or region; VH) and an antibody light chain variable domain (or region; VL) connected by a linker. Such scFv molecules can have the general structures: NH2-VL-linker-VH-COOH or NH2-VH-linker-VL-COOH. ScFvs can be prepared according to methods known in the art (see, for example, Bird et al., (1988) Science 242:423-426 and Huston et al., (1988) Proc. Natl. Acad. Sci. USA 85:5879-5883). The scFv molecules typically comprise a linker (e.g., a Ser-Gly linker). Exemplary suitable linkers are described in, e.g., Holliger et al. (1993), Proc. Natl. Acad. Sci. USA 90:6444-6448; Alfthan et al. (1995), Protein Eng. 8:725-731; Choi et al. (2001), Eur. J. Immunol. 31:94-106; Hu et al. (1996), Cancer Res. 56:3055-3061; Kipriyanov et al. (1999), J. Mol. Biol. 293:41-56, and Roovers et al. (2001), Cancer Immunol.

The term “complementarity determining region” or “CDR” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The positions of the CDRs and framework regions can be determined using various known definitions in the art, e.g., Kabat (“Kabat numbering scheme”), Chothia, IMGT, AbM, and combined definitions (see, e.g., Kabat et al. (1991), “Sequences of Proteins of Immunological Interest,” 5th Ed. Public Health Service, National Institutes of Health, Bethesda, MD; Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Lefranc, M. P., Nucleic Acids Res., 29:207-209 (2001); Al-Lazikani et al., J.Mol.Biol., 273:927-748 (1997)). Definitions of antigen combining sites are also described in the following: Ruiz et al., Nucleic Acids Res., 28:219-221 (2000); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). Under IMGT, the CDR regions of an antibody can be determined using the program IMGT/DomainGap Align.

Under the Kabat numbering scheme, the CDR amino acid residues in the heavy chain variable domain (VH) are typically numbered 31-35 (HCDR1), 50-65 (HCDR2), and 95-102 (HCDR3); and the CDR amino acid residues in the light chain variable domain (VL) are typically numbered 24-34 (LCDR1), 50-56 (LCDR2), and 89-97 (LCDR3). Under the Chothia numbering scheme, the CDR amino acids in the VH are typically numbered 26-32 (HCDR1), 52-56 (HCDR2), and 95-102 (HCDR3); and the CDR amino acid residues in the VL are typically numbered 26-32 (LCDR1), 50-52 (LCDR2), and 91-96 (LCDR3). In a Combined Kabat and Chothia numbering scheme, the CDRs typically correspond to the amino acid residues that are part of a Kabat CDR, a Chothia CDR, or both. For instance, under the Combined numbering scheme, the CDRs typically correspond to amino acid residues 26-35 (HCDR1), 50-65 (HCDR2), and 95-102 (HCDR3) in a human VH; and amino acid residues 24-34 (LCDR1), 50-56 (LCDR2), and 89-97 (LCDR3) in a human VL.

As used herein, the term “EU numbering” or “EU numbering scheme” refers to the EU numbering convention for the constant regions of an antibody, as e.g., described in Edelman, G. M. et al., Proc. Natl. Acad. USA, 63, 78-85 (1969) and Kabat et al., in “Sequences of Proteins of Immunological Interest”, U.S. Dept. Health and Human Services, 5th edition, 1991.

The term “antibody framework” or “framework region” as used herein refers to the part of the variable domain, either VL or VH, which serves as a scaffold for the antigen binding loops (CDRs) of this variable domain.

The term “part antibody” or “half antibody” refers to a portion of an antibody, antibody fragment, antibody-like molecule or multispecific antibody that comprises an antigen-binding domain. In some embodiments, a part or half antibody refers to a heavy and a light chain pair of, for example, an IgG antibody (e.g., IgG1 or IgG4 antibody). In some embodiments, a part or half antibody comprises a polypeptide comprising a VL and a CL domain and a polypeptide comprising a VH domain, a CH1 domain, a hinge domain, a CH2 domain, and a CH3 domain, wherein said VH and VL domains comprise an antigen-binding domain. In some embodiments, a part or half antibody refers to, e.g. a polypeptide comprising a scFv domain and an Fc polypeptide chain (comprising a CH2 domain and a CH3 domain, and optionally a hinge region). In some embodiments, a part or half antibody comprises a Fab, and optionally further comprises an Fc polypeptide.

In some embodiments, a first part or half antibody may be paired with another part or half antibody, such that the first part and second will associate, e.g., heterodimerize. This can, for example, be achieved via association of Fc polypeptide chains comprised by the first and second parts forming an Fc region heterodimer, as described later herein. Advantageously, a first part of half antibody may comprise a lambda (2) light chain (e.g., a lambda 1 light chain), and the second part of half antibody may comprise a kappa light chain (e.g., a kappa 4 light chain), the presence of the lambda and kappa light chains promoting heterodimerization of the first and second part of half antibodies (e.g., as described in WO2023/209568 or WO2018/057955) to form the multispecific, e.g., bispecific, antibody.

In some embodiments, a first part or half antibody is covalently linked to a second part or half antibody. In some embodiments, either a first half antibody, a second part half antibody, or both a first and second half antibody may be linked to an additional antigen binding domain.

Preferably, a “part antibody” or “half antibody” described herein is a “half IgG antibody”. The term “part IgG antibody”, “half IgG antibody” or “half IgG antibody fragment” refers to a heavy and light chain pair of an IgG antibody. In such embodiments, the light chain comprises a VL domain and a CL domain, and the heavy chain comprises a VH domain, a CH1 domain, a hinge domain, a CH2 domain, and a CH3 domain (i.e., an Fd and an Fc), wherein said VL and VH domains comprise an antigen-binding domain. Each part or half IgG antibody described herein preferably comprises an Fc polypeptide chain as described later herein. As with all polypeptide chains, an Fc polypeptide chain contains an N-terminus and a C-terminus, each of which is capable of being linked to an antigen-binding domain. An example of a part or half IgG antibody is a part or half IgG1 antibody or a part or half IgG4 antibody.

As used herein, the term “Fc” or “Fc polypeptide” includes a polypeptide comprising a CH2 and a CH3, and, optionally, any portion of an antibody hinge region. An Fc region is comprised of two Fc polypeptide chains, each comprising a CH2 and a CH3, that dimerize to form the Fc region. In some embodiments, an antibody described herein comprises an Fc polypeptide that is a variant of a human Fc polypeptide, such as a human IgG Fc polypeptide (for example a human IgG1 or IgG4 Fc polypeptide). A variant Fc polypeptide may comprise one or more amino acid modifications (mutations), preferably one or more amino acid substitutions, relative to a reference (“wild-type”) Fc polypeptide, for example an Fc polypeptide comprising the amino acid sequence SEQ ID NO: 59. Examples of mutations include silencing mutations, half-life extension mutations, K-i-H (Knob-in-Hole), and FAE (Fab arm exchange) mutations, discussed later herein.

Each part or half antibody in the cases of multispecific antibodies described herein preferably comprises an Fc polypeptide e.g., a first part antibody binding to IL-18 comprises a heavy chain comprising an Fc polypeptide and a second part antibody binding to IL-13 comprises a heavy chain comprising an Fc polypeptide, allowing for the two Fc polypeptide chains to dimerize into an Fc region of a multispecific (e.g., bispecific) antibody described herein. The dimerization may be promoted by the inclusion of pair K-i-H (Knob-in-Hole) or FAE (Fab arm exchange) mutations in the Fc polypeptides, discussed later herein.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the term antibody is intended to include derivatized and otherwise modified forms of the antibodies described herein, including immuno-adhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag). One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

The term “monospecific antibody,” as used herein, refers to an antibody that binds to a single target antigen. The term “multispecific antibody” as used herein refers to an antibody that binds to two or more different (distinct) antigens or to an antibody that binds to two or more different (distinct) epitopes on the same antigen. Recognition of each antigen is generally accomplished via an antigen-binding domain (e.g., an IL-18 or IL-13 antigen-binding domain).

In some embodiments, the multispecific binding molecules comprise four polypeptide chains that together comprise a plurality, e.g., two or more, e.g., two, three or four, preferably two, antigen-binding domains that bind two or more different antigens. The term “multispecific” thus includes e.g., bispecific, trispecific, and tetraspecific molecules. Preferably, the multispecific antibodies are bispecific. Bispecific IgG molecules are typically bivalent and possess an asymmetric architecture due to the presence of, at least, different Fv regions. Depending on the method of preparation and the origin of the heavy and light chains, they may further differ in the constant regions of the heavy or light chains (e.g., via the introduction of “matching” (or pair) K-i-H or Fab arm exchange (FAE) mutations in the constant region of the heavy chains). A bispecific antibody is typically able to bind two different antigens or epitopes simultaneously or sequentially.

The term “multivalent antibody” as used herein refers to an antibody that has at least two antigen binding domains. In some embodiments, a multivalent antibody is a bivalent antibody, trivalent antibody or a tetravalent antibody. The term “bivalent antibody” as used herein refers to an antibody that has two antigen-binding domains. The terms “trivalent antibody” and “tetravalent antibody” as used herein refer to an antibody that has three antigen-binding domains, or four antigen-binding domains, respectively. The term “monovalent antibody” means that the antibody has one antigen-binding domain.

The term “monovalent with respect to binding IL-18” as used herein means that the antibody has one IL-18 binding domain. The term “monovalent with respect to binding IL-13” as used herein means that the antibody has one IL-13 binding domain. In some preferred aspects, a bispecific antibody is monovalent with respect to binding IL-18 and monovalent with respect to binding IL-13, i.e., has one IL-18 binding domain and one IL-13 binding domain.

The term “monoclonal antibody” as used herein refers to polypeptides, including antibodies, antibody parts, and antigen-binding fragments, that have substantially identical amino acid sequence and/or are derived from the same genetic source. This term also includes preparations of antibody molecules of single molecular composition. A monoclonal antibody composition displays a single binding specificity and affinity for a particular epitope or antigen. Methods for generation of monoclonal antibodies using phage display technology are known in the art (Proetzel, G., Ebersbach, H. (Eds.) Antibody Methods and Protocols. Humana Press ISBN 978-1-61779-930-3; 2012).

The term “humanized” antibody as used herein, refers to an antibody that retains the reactivity of a non-human antibody or antibody variable domain while being less immunogenic in humans. The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik, et al., (2000) J Mol Biol; 296:57-86). The human antibodies may include amino acid residues not encoded by human sequences (e.g., mutations introduced by random or site-specific mutagenesis in vitro or by somatic mutation in vivo).

The term “co-expression” means that different polypeptides are expressed together in a single host cell, common for all the polypeptides. Co-expression of a multispecific, e.g., bispecific, antibody means that the different parts forming the functional multispecific, e.g., bispecific, antibody are expressed in a single, common, host cell. Co-expression may be achieved by incorporating several expression vectors in the expression host cell, such as one for each of the parts of a bispecific antibody, or by incorporating one expression vector encoding all parts of the multispecific antibody.

The term “Fab-arm” refers to one heavy chain-light chain pair of an antibody.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

A “modification” or “mutation” of an amino acid residue/position, as used herein, refers to a change of a primary amino acid sequence as compared to a starting amino acid sequence, wherein the change results from a sequence alteration involving said amino acid residue/positions. The modification may e.g., be a substitution of the residue (or at said position) with another amino acid (e.g., a conservative or non-conservative substitution), an insertion, or a deletion of a residue. A “variant” or “mutant” of a given sequence comprises at least one amino acid modification relative to said sequence. In some embodiments, the amino acid modification is an amino acid substitution.

In some embodiments, an amino acid modification is a conservative amino acid substitution. For polypeptide sequences, “conservatively modified variants” include individual substitutions, deletions or insertions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosed sequences. The following exemplary eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)). In some embodiments, the term “conservative sequence modifications” or “conservative modifications” is used to refer to amino acid modifications that do not significantly affect or alter the binding characteristics of the antibody containing the amino acid sequence.

The term “half-life” as used herein, refers to the time required for the concentration of an antibody to fall by one-half (Rowland M and Towzer T N: Clinical Pharmacokinetics. Concepts and Applications. Third edition (1995); and Bonate P L and Howard D R (Eds): Pharmacokinetics in Drug Development, Volume 1 (2004)). Further details may also be found in Kenneth, A et al: Chemical Stability of Pharmaceuticals: A Handbook for Pharmacists and in Peters et al, Pharmacokinetic analysis: A Practical Approach (1996). Reference is also made to “Pharmacokinetics”, M Gibaldi & D Perron, published by Marcel Dekker, 2nd Rev. ex edition (1982), which describes pharmacokinetic parameters such as alpha half-life and beta half-life and area under the curve (AUC). Preferably, all pharmacokinetic parameters and values quoted herein are to be read as being values in a human. Preferably, the term “half-life” as used herein refers to “in vivo half-life”, i.e., the half-life to the antibody circulating in the blood or serum of a given mammal, preferably a human.

Multispecific, e.g., bispecific, antibodies comprising a first part that binds to IL-18 and a second part that binds to IL-13, and antigen-binding fragments thereof described herein, are heterodimers, which means that the first part binding to IL-18 comes from a first antibody (binding to IL-18) and the second part binding to IL-13 comes from a second antibody (binding to IL-13). A “heterodimerization modification” is a modification to one or both parts of the antibodies forming the antibody, intended to facilitate such formation. Examples of heterodimerization modifications are the K-i-H and FAE mutations discussed above, and further described later herein.

As used herein, “identity” refers to the sequence matching between two polypeptides, molecules or between two nucleic acids. When a respective position in both of the two compared sequences is occupied by the same base or amino acid (for instance, if a position in each of two polypeptides is occupied by a lysine), then the respective molecules are identical at that position.

The “percentage identity” or “percent identity” between two sequences is a function of the number of identical positions shared by the sequences, taking into account the number of gaps, and the length of each gap, which need to be introduced for optimal alignment of the two sequences. Generally, a comparison is made when two sequences are aligned to give maximum identity. Such alignment can be provided using, for instance, the method of the Needleman and Wunsch (J. Mol. Biol. (48): 444-453 (1970)) algorithm which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. As another example, the percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. One suitable set of parameters are a Blossum 62 scoring matrix with a gap penalty of 12, a gap extend penalty of 4, and a frameshift gap penalty of 5.

The percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm. Two sequences are “identical” if they have the same sequence of amino acids or nucleotides over the region being compared. Two sequences are “substantially identical” if two sequences have a specified percentage of amino acid residues or nucleotides that are the same (i.e., 80% identity, optionally 85%, 90%, 95%, 98% or 99% identity over a specified region, or, when not specified, over the entire sequence), when compared and aligned for maximum correspondence over a comparison window, or designated region as measured using one of the sequence comparison algorithms discussed herein or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A “comparison window,” as used herein, includes reference to a segment corresponding to a number of contiguous positions in which a sequence may be compared to a reference sequence of the same number of contiguous positions after the two sequences are optimally aligned. The percent identity is typically calculated by comparing sequences of similar lengths.

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information, and be e.g., run using their default parameters. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. Other than percentage of sequence identity noted above, another indication that two nucleic acid sequences or polypeptides are substantially identical may be that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions.

In some embodiments, sequence identity is calculated over the entire length of two sequences that are being compared.

The term “isolated antibody” refers to an antibody that is removed from its original or native environment (e.g., the natural environment if it is naturally occurring). Moreover, an isolated antibody may be substantially free of other cellular material and/or chemicals, for example, an antibody isolated from a cell culture supernatant. Such an antibody may be part of a composition (e.g., pharmaceutical composition), and still be considered isolated in that such a composition is not part of the environment in which it was originally or natively found. Methods of isolation are known in the art and include electrophoretic (e.g., SDS-PAGE, isoelectric focusing, capillary electrophoresis), chromatographic (e.g., ion-exchange, reverse phase HPLC), and filtration methods. In some embodiments, the antibody has been isolated and purified to greater than 95% or than about 95%, greater than 96% or than about 96%, greater than 97% or than about 97%, greater than 98% or than about 98%, or greater than 99% or than about 99% purity as determined by any suitable method in the art (e.g., the electrophoretic or chromatographic methods discussed above; see also Flatman et al., 2007, J Chromatogr B Analyt Technol Biomed Life Sci, 848:79-87). Purity may also be e.g., determined using an intact UPLC-MS mass screening approach, e.g., following protein-A purification, or using an LC-MS approach. Purity may also be e.g., determined using size exclusion chromatography (SEC), e.g., on commercial columns like the BEH200 SEC column (Waters #186005225). Purity may also be e.g., determined using CE-SDS capillary electrophoresis, e.g., on commercial systems like the Beckman Coulter PA 800 system (Beckman Coulter, CA, USA).

The term “epitope” refers to a site on IL-18 and/or IL-13 to which an antibody of the disclosure interacts with and includes linear, discontinuous, or conformational epitopes. Epitopes can be formed both from contiguous amino acids and/or non-contiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, 15, 16, 17, 18, 19, 20, 21, 22, 23, 24, 25, 26, 27, 28, 29, or 30 amino acids in a unique spatial conformation. Methods of determining spatial conformation of epitopes include techniques in the art, for example, x-ray crystallography and 2-dimensional nuclear magnetic resonance (see, e.g., Epitope Mapping Protocols in Methods in Molecular Biology, Vol. 66, G. E. Morris, Ed. (1996)), or electron microscopy. A “paratope” is the part of the antibody which recognizes the epitope of the antigen.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. Degenerate codon substitutions may e.g., be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994). The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as a retroviral vector, a lentiviral vector, an adenoviral vector, or an adeno-associated viral vector (AAV), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, lentiviruses, adenoviruses and adeno-associated viruses), which serve equivalent functions.

The term “host cell” refers to a cell into which a recombinant nucleic acid molecule or expression vector has been introduced, e.g., a nucleic acid molecule or expression vector encoding a multispecific, e.g., bispecific, antibody or part thereof described herein. Such a cell may alternatively be referred to as “recombinant host cell” or “engineered host cell”.

As used herein, the term “protein” refers to any organic compound made of amino acids arranged in one or more linear chains and folded into a globular form. The amino acids in a polymer chain are joined together by the peptide bonds between the carboxyl and amino groups of adjacent amino acid residues. The term “protein” further includes, without limitation, peptides, polypeptides, single chain polypeptides or any complex molecules consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation and the like, incorporation of non-natural amino acids, and amino acid modification for chemical conjugation with another molecule. Unless otherwise indicated, a particular polypeptide sequence also implicitly encompasses conservatively modified variants thereof.

The term “subject in need” includes human and non-human animals. Non-human animals include all vertebrates (e.g.: mammals and non-mammals) such as, non-human primates (e.g.: cynomolgus monkey), sheep, dog, cow, chickens, amphibians, and reptiles. Except when noted, the terms “patient” or “subject in need” are used herein interchangeably. In a preferred embodiment, the “subject in need” or “patient” is a human. As used herein, the terms “cyno” or “cynomolgus” refer to the cynomolgus monkey (Macaca fascicularis). The term “subject in need thereof” with respect to the therapies of the invention refers to a subject, preferably a human, that could benefit from administration of a therapy described herein, e.g., that exhibits one or more symptoms or indicia of atopic dermatitis or a related condition and/or that has been diagnosed with atopic dermatitis or a related condition. In some embodiments, the therapies described herein may be used to treat patients that show elevated levels of one or more AD-associated biomarkers (described elsewhere herein). In some embodiments, the methods and uses provided herein comprise administering a pharmaceutical composition comprising a multispecific, e.g., bispecific, antibody binding to IL-18 and IL-13 (or antigen-binding fragment thereof) to patients with elevated levels of CCL17 (TARC), IL-13, IL-18, IL-18BP, pSTAT6, and/or IFNγ, as compared to a subject not diagnosed with atopic dermatitis or a related condition. In some embodiments, the methods and uses provided herein comprise administering a pharmaceutical composition comprising a multispecific, e.g., bispecific, antibody binding to IL-18 and IL-13 (or antigen-binding fragment thereof) to patients with abnormal or aberrant levels of a biomarker selected from the group consisting of CCL17 (TARC), pSTAT6, IFNγ, IL-13 (bioactive), IL-18 (bioactive) (e.g., serum free IL-18), IL-18BP (e.g., serum IL-18BP), IgE (e.g., serum IgE), CCL26 (eotaxin-3), CCL22 (MDC2), hsCRP, CD40, IL-24, and IL-22, as compared to a subject not diagnosed with atopic dermatitis or a related condition.

The terms “therapy”, “treat”, “treating” or “treatment” include therapeutic treatments, prophylactic treatments and applications in which one reduces the risk that a subject will develop a disorder or other risk factor. Treatment does not require the complete curing of a disorder and encompasses the reduction of the symptoms or underlying risk factors. The invention further relates to uses or methods of treatment of AD, e.g., mild, moderate-to-severe, or AD, wherein the treatment comprises treating or alleviating one or more symptoms of AD. Thus, as used herein, the terms “therapy”, “treat”, “treatment” and “treating” refer to the reduction or amelioration of the progression, severity and/or duration of the disease, or the amelioration or alleviation of one or more symptoms, suitably of one or more discernible symptoms of the disease, resulting from the administration of the pharmaceutical composition comprising a multispecific, e.g., bispecific, antibody binding to IL-18 and IL-13 or antigen-binding fragment thereof. In specific embodiments, the terms “therapy”, “treat”, “treatment” and “treating” refer to the amelioration or alleviation of at least one measurable physical parameter of a disease, e.g., AD, wherein the physical parameter is not necessarily discernible by the patient. Subjects in which the abovementioned reduction, amelioration, or alleviation is obtained can be considered to have a positive response to the treatment. “Prevention” as used in this context refers to methods which aim to prevent the onset of a disease or its symptoms or which delay the onset of a disease or its symptoms.

The term “dose” refers to the quantity of a pharmaceutical composition or multispecific, such as bispecific, antibody (or antigen-binding fragment thereof) described herein, which is administered to a subject.

The term “therapeutically effective” or “therapeutically effective amount” or “therapeutically efficacious dose” means a sufficient amount, e.g. of an antibody or composition described herein, or other pharmaceutically active agent, to provide the desired therapeutic effect (i.e., a reduction in disease activity, reduction in disease progression, reduction in disease signs and/or symptoms, etc.). A therapeutically effective amount is also one in which any toxic or detrimental effects resulting from administration are outweighed by the therapeutically beneficial effects.

In some embodiments, “therapeutically effective amount” may refer to the amount of multispecific, e.g., bispecific, antibody binding to IL-18 and IL-13 (or antigen-binding fragment thereof) described herein, the administration of which achieves a specified result, for example, an improvement in an AD-associated parameter, e.g., a decrease in Investigator's Global Assessment (IGA) score, a decrease in Body Surface Area Involvement of Atopic Dermatitis (BSA) score, a decrease in Eczema Area and Severity Index (EASI) score, a decrease in Dermatology Life Quality Index (DLQI) score, and/or a decrease in Peak Pruritus Numeric Rating scale (PP-NRS) score. In some embodiments, “therapeutically effective amount” may refer to the amount of multispecific, e.g., bispecific, antibody binding to IL-18 and IL-13 (or antigen-binding fragment thereof) described herein, the administration of which achieves, for example, an improvement in the level of one or more AD-associated biomarkers, e.g., one or more AD-associated biomarkers selected from the group consisting of CCL17 (TARC), pSTAT6, IFNγ, IL-13 (bioactive), IL-18 (bioactive) (e.g., serum free IL-18), and IL-18BP (e.g., serum IL-18BP), as compared to the level prior to treatment with the multispecific antibody or antigen-binding fragment thereof (baseline). In some embodiments, the one or more AD-associated biomarkers are selected from the group consisting of CCL17 (TARC), pSTAT6, IFNγ, IL-13 (bioactive), IL-18 (bioactive) (e.g., serum free IL-18), IL-18BP (e.g., serum IL-18BP), IgE (e.g., serum IgE), CCL26 (eotaxin-3), CCL22 (MDC2), hsCRP, CD40, IL-24, and IL-22.

The term “prophylactically effective” amount means a sufficient amount to provide a desired prophylactic effect. Typically, since a prophylactic dose is used in subjects prior to or at an earlier stage of disease, the prophylactically effective amount will be less than the therapeutically effective amount. Therapeutically effective doses of the multispecific, e.g., bispecific, antibodies (or antigen-binding fragments thereof) of the disclosure are described later herein.

The term “atopic dermatitis”, “AD”, or “eczema”, as used herein, means an inflammatory skin disease characterized by intense pruritus (e.g., severe itch) and by scaly and dry eczematous lesions. The term “atopic dermatitis” or “eczema” includes, but is not limited to, AD (eczema) caused by or associated with epidermal barrier dysfunction, allergy (e.g., skin allergy, allergy to certain foods, pollen, mold, dust mite, animals, etc.), radiation exposure, and/or asthma. The present disclosure encompasses methods to treat patients with mild, moderate-to-severe, or severe AD. As used herein, “moderate-to-severe AD”, is characterized by intensely pruritic, widespread skin lesions that are often complicated by persistent bacterial, viral or fungal infections. Moderate-to-severe AD also includes chronic AD in patients. In many cases, the chronic lesions include thickened plaques of skin, lichenification and fibrous papules. Patients affected by moderate-to-severe AD also, in general, have 10% or more, 15% or more, or 20% or more of the body's skin surface area (BSA) affected, or at least 10% of skin area in addition to involvement of the eyes, hands and body folds. Patients affected by moderate-to-severe AD, in general, have (i) an Investigator's Global Assessment (IGA) score of at least 3, for example 3 or 4, (ii) an Eczema Area and Severity Index (EASI) score of at least 10, for example at least 12 or at least 16, and/or (iii) itch. Moderate-to-severe AD is also considered to be present in patients who require frequent treatment with topical corticosteroids. In some embodiments, atopic dermatitis is classified as moderate-to-sever by having a Rajka/Langeland criteria score (e.g., described in Rajka & Langeland, 1989, Acta Derm Venereol Suppl 144:13-14) from 4.5 to 9.

In certain embodiments provided herein are methods to treat both the extrinsic and the intrinsic forms of AD. The extrinsic form of AD is associated with IgE-mediated sensitization and increased levels of Th2 cytokines any typically involves 70% to 80% of patients with AD. The intrinsic form without IgE-mediated sensitization typically involves 20% to 30% of patients with AD; these patients have lower levels of IL-4 and IL-13 than extrinsic AD.

Individuals with atopic dermatitis have an increased risk of developing one or more related conditions, typically related to inflammation. Thus, the term “related condition”, as used herein, refers particularly to conditions including those related to inflammation, such as allergy (e.g., skin allergy, allergy to certain foods, pollen, mold, dust mite, animals, etc.), asthma (such as allergic and non-allergic asthma); infectious disorders, skin infection, cutaneous infection, eczema herpeticum, erysipelas, cellulitis, extra-cutaneous infection, encephalitis, endocarditis, infectious arthropathy, enterocolitis, septicemia, respiratory infection, upper respiratory tract infection, lower respiratory tract infection, lung infection, cardiac infection, brain infection, bone infection, gastrointestinal infection, increased colonization with Staphylococcus aureus in lesional and non-lesional skin, autoimmune disorders, respiratory disorders, neuropsychiatric disorders, musculoskeletal disorders, and cardiovascular disorders associated with atopic dermatitis.

A skin infection, in particular a bacterial skin infection, is common in AD and is in part due to breaks in the skin from very dry, split skin and from scratching the itchy areas. In certain embodiments provided herein are methods to treat AD, which is accompanied by an infection, in particular a skin infection, more particularly a skin superinfection. In certain embodiments provided herein are methods to treat AD related conditions, wherein the related condition is an infection, in particular a skin infection, more particularly a skin superinfection. In certain embodiments, the infection is (i) a bacterial infection, e.g., caused by staphylococcal bacteria, such as Staphylococcus aureus or Staphylococcus epidermidis, or streptococcal bacteria, and/or (ii) a viral infection, e.g., herpes viral infection. The term “superinfection” as used herein refers to a secondary infection superimposed on an already affected lesional tissue. Superinfection complicates skin lesions and/or leads to colonization or infection by bacteria including commensals. Superinfection could be a viral or a bacterial infection, which is in part due to breaks in the skin from very dry, split skin and from scratching the itchy areas.

The terms “pruritus” and “itch”, which are used synonymously herein, refer to a sensation that causes the desire or reflex to scratch the skin. Scratching can be problematic particularly in the case of chronic skin diseases that induce itching, such as atopic dermatitis, because the permanent itching stimulates patients to scratch the affected skin areas constantly and/or excessively, which often leads to skin injuries and further deterioration of the skin surface, as well as sleep deprivation, depression and low quality of life.

Various aspects of the invention are described in further detail in the following sections and subsections.

### Multispecific Antibody and Antigen-Binding Fragments Thereof

The pharmaceutical compositions described herein comprise multispecific, e.g., bispecific, antibodies or antigen-binding fragments thereof as an active ingredient.

In an aspect, there is provided a pharmaceutical composition comprising a multispecific, e.g., bispecific, antibody or antigen binding fragment thereof, wherein the multispecific antibody comprises:
i) a first part that binds to IL-18, and;
ii) a second part that binds to a target other than IL-18,
wherein the pharmaceutical composition comprises from about 15 mg/mL to about 200 mg/mL of the multispecific antibody or antigen-binding fragment thereof.

In an aspect, there is provided a pharmaceutical composition comprising a multispecific, e.g., bispecific, antibody or antigen binding fragment thereof, wherein the multispecific antibody comprises:
i) a first part that binds to a target other than IL-13, and;
ii) a second part that binds to IL-13,
wherein the pharmaceutical composition comprises from about 15 mg/mL to about 200 mg/ml of the multispecific antibody or antigen-binding fragment thereof.

A preferred multispecific, e.g., bispecific, antibody comprises a first part that binds to IL-18 and a second part that binds to IL-13.

Accordingly, in an aspect, there is provided a pharmaceutical composition comprising a multispecific, preferably bispecific, antibody or antigen-binding fragment thereof, wherein the multispecific antibody comprises:
i) a first part that binds to IL-18, and;
ii) a second part that binds to IL-13,
wherein the pharmaceutical composition comprises from about 15 mg/mL to about 200 mg/mL of the multispecific antibody or antigen-binding fragment thereof.

In some embodiments, a pharmaceutical composition described herein has a pH from about 5 to about 8. In some embodiments, a pharmaceutical composition described herein has a pH of from about 5.5 to about 7.4. In some embodiments, a pharmaceutical composition described herein has a pH from about 6 to about 7, e.g., about 6.5. Preferably, the pharmaceutical composition is a liquid (e.g., stable liquid) pharmaceutical composition, e.g., an aqueous pharmaceutical composition.

A first part that binds to IL-18 can comprise an IL-18 binding domain, and a second part that binds to IL-13 can comprise an IL-13 binding domain. More information on IL-18 and IL-13 binding sequences is provided later herein.

Preferably, the IL-18 and IL-13 are human.

Multispecific antibodies and antigen-binding fragments thereof described herein may be multivalent and/or monovalent with respect to binding each antigen. Bispecific antibodies, for example, include but are not limited to bivalent (monovalent with respect to binding each antigen), trivalent (bivalent with respect to binding one antigen and monovalent with respect to binding the other antigen), or tetravalent (bivalent with respect to binding both antigens). In preferred embodiments, the multispecific antibody is bispecific. In other preferred embodiments, the bispecific antibody is bivalent and is monovalent with respect to binding IL-18 and monovalent with respect to binding IL-13.

Non-limiting exemplary multispecific antibody formats include, but are not limited to, bivalent bispecific F (ab) 2 format, bivalent bispecific Fab-scFv format, bivalent bispecific scFv-scFv format, bivalent bispecific IgG format, trivalent bispecific IgG format e.g., a whole antibody molecule specific to one antigen having e.g., a single chain Fab (scFab) linked to the C-terminal end of the molecule specific to another antigen, tetravalent bispecific IgG format, trivalent bispecific scFv3 format, trivalent bispecific (scFv) 2-Fab format, trivalent bispecific F (ab) 3 format, trivalent trispecific scFv3 format, trivalent trispecific Fab-scFv2 format (e.g., a Fab fragment specific to one antigen C-terminally linked to two scFvs specific to another antigen, one on each chain), trivalent trispecific F (ab) 3 format, or trivalent trispecific IgG format, or tetravalent trispecific IgG format. Other exemplary formats incorporate sdAbs or fragments thereof such as a VHH, for example a Fab-VHH or scFv-VHH format. Optionally, the multispecific antibody may be incorporated into an antibody-drug conjugate.

In some embodiments, the multispecific, preferably bispecific, antibody comprises a part antibody, scFv, Fab, or half IgG antibody, or antigen-binding fragments thereof, having binding specificity for IL-18; and one or more of a part antibody, scFv, Fab, or half IgG antibody, or antigen-binding fragments thereof, having binding specificity for IL-13. An anti-IL-18 half IgG antibody comprises a polypeptide comprising a VL domain and a CL domain, and a second polypeptide comprising a VH domain, a CH1 domain, a hinge domain, a CH2 domain, and a CH3 domain (i.e., an Fd and an Fc), wherein said VL and VH domains comprise an anti-IL-18 antigen-binding domain. An anti-IL-13 half IgG antibody comprises a polypeptide comprising a VL domain and a CL domain, and a second polypeptide comprising a VH domain, a CH1 domain, a hinge domain, a CH2 domain, and a CH3 domain (i.e., an Fd and an Fc), wherein said VL and VH domains comprise an anti-IL-13 antigen-binding domain.

In preferred embodiments, the multispecific, preferably bispecific, antibody is an IgG1 antibody comprising an anti-IL-18 half antibody paired with an anti-IL-13 half antibody.

Protocols for generating multispecific e.g., bispecific/heterodimeric antibodies are available in the art; including but not limited to, for example, the “knob in a hole” approach described in, e.g., U.S. Pat. No. 5,731,168; the electrostatic steering Fc pairing as described in, e.g., WO2009/089004, WO2006/106905 and WO2010/129304; Strand Exchange Engineered Domains (SEED) heterodimer formation as described in, e.g., WO2007/110205; Fab arm exchange as described in, e.g., WO2008/119353, WO2011/131746, and WO2013/060867; double antibody conjugate, e.g., by antibody cross-linking to generate a bispecific structure using a heterobifunctional reagent having an amine-reactive group and a sulfhydryl reactive group as described in, e.g., U.S. Pat. No. 4,433,059; bispecific antibodies generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono-and oligovalent receptors, e.g., VH-CH1 regions (Fd regions) of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multi-specific binding proteins, e.g., dimer of polypeptides having first domain with binding region of Ig heavy chain variable region, and second domain with binding region of Ig light chain variable region, generally termed diabodies (higher order structures are also encompassed creating for bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VL and VH domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; string of VH domains (or VL domains in family members) connected by peptide linkages with crosslinkable groups at the C-terminus further associated with VL domains to form a series of FVs (or scFvs), as described in, e.g., U.S. Pat. No. 5,864,019; VL and VH domains, scFvs, or Fabs wherein one of the antigens is bound monovalently and one of the antigens is bound bivalently, optionally comprising heterodimeric Fc regions, as described in, e.g., WO2011/028952; and single chain binding polypeptides with both a VL and VH domain linked through a peptide linker are combined into multivalent structures through non-covalent or chemical crosslinking to form, e.g., homobivalent, heterobivalent, trivalent, and tetravalent structures using both scFv or diabody type format, as described in, e.g., U.S. Pat. No. 5,869,620. A multispecific, e.g., bispecific, antibody or antigen-binding fragment thereof binding to IL-18 and IL-13 may preferably be generated as described in WO2023/209568.

The amino acid sequences shown in Table 1 are non-limiting examples of sequences that can be comprised by the multispecific, preferably bispecific, antibodies described herein, as well as by antigen-binding fragments thereof.

In some embodiments, the multispecific, preferably bispecific, antibody or antigen-binding fragment thereof comprises:
i) a first part that binds to IL-18 comprising:
an HCDR1 comprising SEQ ID NO: 29, SEQ ID NO: 32, SEQ ID NO: 35, or SEQ ID NO: 38;
an HCDR2 comprising SEQ ID NO: 30, SEQ ID NO: 33, SEQ ID NO: 36, or SEQ ID NO: 39;
an HCDR3 comprising SEQ ID NO: 31, SEQ ID NO: 34, SEQ ID NO: 37, or SEQ ID NO: 40;
an LCDR1 comprising SEQ ID NO: 1, SEQ ID NO: 4, SEQ ID NO: 7, or SEQ ID NO: 10;
an LCDR2 comprising SEQ ID NO: 2, SEQ ID NO: 5, or amino acid sequence RNN, and;
an LCDR3 comprising SEQ ID NO: 3, SEQ ID NO: 6, SEQ ID NO: 9, or SEQ ID NO: 12, and;
ii) a second part that binds to IL-13 comprising:
an HCDR1 comprising SEQ ID NO: 43, SEQ ID NO: 46, SEQ ID NO: 49, or SEQ ID NO: 52;
an HCDR2 comprising SEQ ID NO: 44, SEQ ID NO: 47, SEQ ID NO: 50, or SEQ ID NO: 53;
an HCDR3 comprising SEQ ID NO: 45, SEQ ID NO: 48, SEQ ID NO: 51, or SEQ ID NO: 54;
an LCDR1 comprising SEQ ID NO: 15, SEQ ID NO: 18, SEQ ID NO: 21, or SEQ ID NO: 24;
an LCDR2 comprising SEQ ID NO: 16, SEQ ID NO: 19, or amino acid sequence LAS, and;
an LCDR3 comprising SEQ ID NO: 17, SEQ ID NO: 20, SEQ ID NO: 23, or SEQ ID NO: 26.

In some embodiments, the multispecific, preferably bispecific, antibody or antigen-binding fragment thereof comprises:
i) a first part that binds to IL-18 comprising:
an HCDR1 comprising SEQ ID NO: 29;
an HCDR2 comprising SEQ ID NO: 30;
an HCDR3 comprising SEQ ID NO: 31;
an LCDR1 comprising SEQ ID NO: 1;
an LCDR2 comprising SEQ ID NO: 2;
an LCDR3 comprising SEQ ID NO: 3, and;
ii) a second part that binds to IL-13 comprising:
an HCDR1 comprising SEQ ID NO: 43;
an HCDR2 comprising SEQ ID NO: 44;
an HCDR3 comprising SEQ ID NO: 45;
an LCDR1 comprising SEQ ID NO: 15;
an LCDR2 comprising SEQ ID NO: 16, and;
an LCDR3 comprising SEQ ID NO: 17,
   preferably wherein the HCDR1, HCDR2, HCDR3, LCDR1, LCDR2, and LCDR3 are defined according to the Combined (Kabat and Chothia) numbering scheme.

In preferred embodiments, the multispecific, preferably bispecific, antibody or antigen-binding fragment thereof comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20,
preferably wherein the HCDR1, HCDR2, HCDR3, LCDR1, LCDR2, and LCDR3 are defined according to the Kabat numbering scheme.

In some embodiments, the multispecific, preferably bispecific, antibody or antigen-binding fragment thereof comprises:
i) a first part that binds to IL-18 comprising:
an HCDR1 comprising SEQ ID NO: 35;
an HCDR2 comprising SEQ ID NO: 36;
an HCDR3 comprising SEQ ID NO: 37;
an LCDR1 comprising SEQ ID NO: 7;
an LCDR2 comprising the amino acid sequence RNN;
an LCDR3 comprising SEQ ID NO: 9, and;
ii) a second part that binds to IL-13 comprising:
an HCDR1 comprising SEQ ID NO: 49;
an HCDR2 comprising SEQ ID NO: 50;
an HCDR3 comprising SEQ ID NO: 51;
an LCDR1 comprising SEQ ID NO: 21;
an LCDR2 comprising amino acid sequence LAS, and;
an LCDR3 comprising SEQ ID NO: 23,
   preferably wherein the HCDR1, HCDR2, HCDR3, LCDR1, LCDR2, and LCDR3 are defined according to the Chothia numbering scheme.

In some embodiments, the multispecific, preferably bispecific, antibody or antigen-binding fragment thereof comprises:
i) a first part that binds to IL-18 comprising:
an HCDR1 comprising SEQ ID NO: 38;
an HCDR2 comprising SEQ ID NO: 39;
an HCDR3 comprising SEQ ID NO: 40;
an LCDR1 comprising SEQ ID NO: 10;
an LCDR2 comprising the amino acid sequence RNN;
an LCDR3 comprising SEQ ID NO: 12, and;
ii) a second part that binds to IL-13 comprising:
an HCDR1 comprising SEQ ID NO: 52;
an HCDR2 comprising SEQ ID NO: 53;
an HCDR3 comprising SEQ ID NO: 54;
an LCDR1 comprising SEQ ID NO: 24;
an LCDR2 comprising amino acid sequence LAS, and;
an LCDR3 comprising SEQ ID NO: 26,
   preferably wherein the HCDR1, HCDR2, HCDR3, LCDR1, LCDR2, and LCDR3 are defined according to the IMGT numbering scheme.

In some embodiments:
the first part that binds to IL-18 comprises a first variable heavy region (VH1) comprising or consisting of SEQ ID NO: 41, or comprising or consisting of a sequence having at least 80%, at least 85%, at least 90%, at least 95%, at least 96%, at least 97%, at least 98%, or at least 99% sequence identity to SEQ ID NO: 41, and a first variable light region (VL1) comprising or consisting of SEQ ID NO: 13, or comprising or consisting of a sequence having at least 80%, at least 85%, at least 90%, at least 95%, at least 96%, at least 97%, at least 98%, or at least 99% sequence identity to SEQ ID NO: 13, and;
the second part that binds to IL-13 comprises a second variable heavy region (VH2) comprising or consisting of SEQ ID NO: 55, or comprising or consisting of a sequence having at least 80%, at least 85%, at least 90%, at least 95%, at least 96%, at least 97%, at least 98%, or at least 99% sequence identity to SEQ ID NO: 55, and a second variable light region (VL2) comprising or consisting of SEQ ID NO: 27, or comprising or consisting of a sequence having at least 80%, at least 85%, at least 90%, at least 95%, at least 96%, at least 97%, at least 98%, or at least 99% sequence identity to SEQ ID NO: 27.

In some embodiments, the first part that binds to IL-18 comprises a first light chain (LC1) that is a lambda light chain, e.g., a lambda 1 light chain, and the second part that binds to IL-13 comprises a second light chain (LC2) that is a kappa light chain, e.g., a kappa 4 light chain. Preferably, the first light chain (LC1) comprises or consists of SEQ ID NO: 14, or comprises or consists of a sequence having at least 80%, at least 85%, at least 90%, at least 95%, at least 96%, at least 97%, at least 98%, or at least 99% sequence identity to SEQ ID NO: 14, and the second light chain (LC2) comprises or consists of SEQ ID NO: 28, or comprises or consists of a sequence having at least 80%, at least 85%, at least 90%, at least 95%, at least 96%, at least 97%, at least 98%, or at least 99% sequence identity to SEQ ID NO: 28.

In some embodiments, the first part that binds to IL-18 is chimeric, humanized, or human, preferably is human. In some embodiments, the second part that binds to IL-13 is chimeric, humanized, or human, preferably is humanized.

In some embodiments, the multispecific, preferably bispecific, antibody or antigen-binding fragment thereof comprises an Fc region. An exemplary such region can be formed by dimerization of a first Fc polypeptide comprised by the first part that binds to IL-18, and of a second Fc polypeptide comprised by the second part that binds to IL-13 as described herein.

Accordingly, in some embodiments,
the first part that binds to IL-18 comprises a first Fc polypeptide, and
the second part that binds to IL-13 comprises a second Fc polypeptide, wherein the first and second Fc polypeptides are associated forming an Fc region.

In some embodiments, the first and/or second Fc polypeptide comprises the amino acid sequence SEQ ID NO: 59 or a variant thereof. It is understood that an Fc polypeptide comprising a variant of SEQ ID NO: 59 maintains the ability (or has enhanced ability) to form an Fc region upon dimerization with another Fc polypeptide.

It is understood that the first Fc polypeptide comprised by the first part and the second Fc polypeptide comprised by the second part of a multispecific, preferably bispecific, antibody described herein may differ in at least one amino acid residue. The first and/or second Fc polypeptide comprised by the first and second parts of the antibody may contain modifications (e.g., amino acid substitutions) e.g., to extend half-life, silence effector function, and/or to facilitate dimerization of Fc polypeptides to an Fc region (e.g., by introduction of K-i-H substitutions or FAE substitutions).

The amino acid substitutions described herein can be introduced into the Fc polypeptides using techniques which are available in the art. Typically, the DNA encoding the heavy chain(s) is genetically engineered using standard techniques, e.g., described in Directed Mutagenesis: a Practical Approach (M J McPherson (ed.), 1991, IRL Press). Oligonucleotide-mediated mutagenesis is an exemplary method for preparing substitution variants of the DNA encoding the heavy chains. This technique is known in the art as e.g., described by Adelman et al., (1983) DNA, 2:183.

The skilled person understands that in cases of a multispecific antibody comprising a first part binding to IL-18 and a second part binding to IL-13 as described herein, the first part may in some embodiments comprise a heavy chain comprising a “hole” mutation in its constant region and the second part may comprise a heavy chain comprising a “knob” mutation in its constant region, or vice versa (the first part comprising the “knob” mutation and the second part comprising the “hole” mutation). Likewise, in the case of FAE mutations, the first part may in some embodiments comprise a heavy chain comprising a K409R or an F405L mutation in its constant region (EU numbering), and the second part may comprise a heavy chain comprising the respective “matching” (or pair) mutation (F405L or K409R as appropriate) in its constant region.

Knobs-into-holes is a well-known heterodimerization technology used for the manufacture of antibodies, particularly multispecific (e.g., bispecific) antibodies. Introduction of either a “knob” or a “hole” in each antibody, for example in the CH3 region of the heavy chain of each antibody promotes the heterodimerization. The engineered antibodies having a “knob” preferentially partner with the engineered antibodies having a “hole”. This method is particularly effective for the production of IgG, for example IgG1 or IgG4, bispecific antibodies wherein production of a “knob” half IgG antibody preferentially partners with the “hole” half IgG antibody.

As the term is used herein, a “knob” refers to at least one amino acid side chain which projects from the interface of a first polypeptide of the multispecific, preferably bispecific, antibody or antigen-binding fragment thereof comprising a heavy chain constant domain and is therefore positionable in a compensatory “hole” in the interface with a second polypeptide of the multispecific, preferably bispecific, antibody or antigen-binding fragment thereof comprising a heavy chain constant domain so as to stabilize the heterodimerization of polypeptide chains (e.g., of the half antibodies) of multispecific antibodies or antigen-binding fragments thereof over homomultimer formation, for example. The knob may exist in the original interface or may be introduced synthetically (e.g. by altering nucleic acid encoding the interface). Typical import residues for the formation of a knob are generally naturally occurring amino acid residues and are typically selected from arginine (R), phenylalanine (F), tyrosine (Y) and tryptophan (W). Most typical are tryptophan and tyrosine. In some embodiments, the original residue for the formation of the protuberance has a small side chain volume, such as alanine, asparagine, aspartic acid, glycine, serine, threonine or valine. A “hole” refers to at least one amino acid side chain which is recessed from the interface of a second polypeptide of the multispecific, preferably bispecific, antibody or antigen-binding fragment thereof comprising a heavy chain constant domain and therefore accommodates a corresponding knob on the adjacent interfacing surface of a first polypeptide of the multispecific, preferably bispecific, antibody or antigen-binding fragment thereof comprising a heavy chain constant domain. The hole may exist in the original interface or may be introduced synthetically (e.g. by altering nucleic acid encoding the interface). The typical import residues for the formation of a hole are usually naturally occurring amino acid residues and are typically selected from alanine (A), serine(S), threonine (T) and valine (V). Most typical are serine, alanine or threonine. In some embodiments, the original residue for the formation of the hole has a large side chain volume, such as tyrosine, arginine, phenylalanine or tryptophan.

In any of the embodiments described herein, the K-i-H CH3 variant domains may be additionally mutated to introduce a pair of cysteine residues. Without being bound by theory, it is believed that the introduction of a pair of cysteine residues capable of forming a disulfide bond provide stability to the heterodimerized antibody.

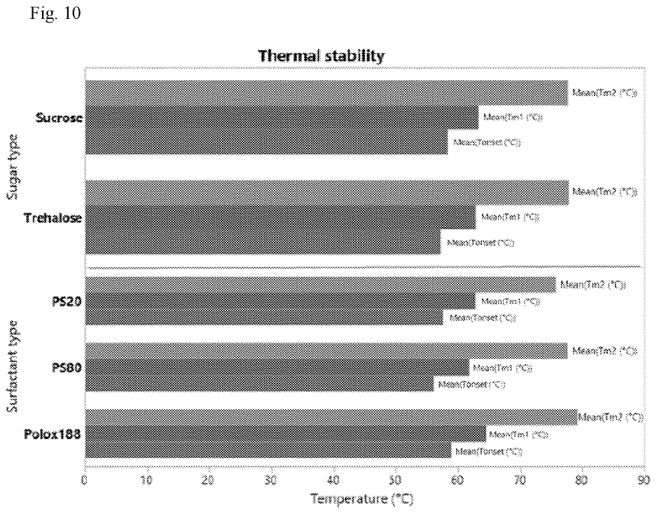

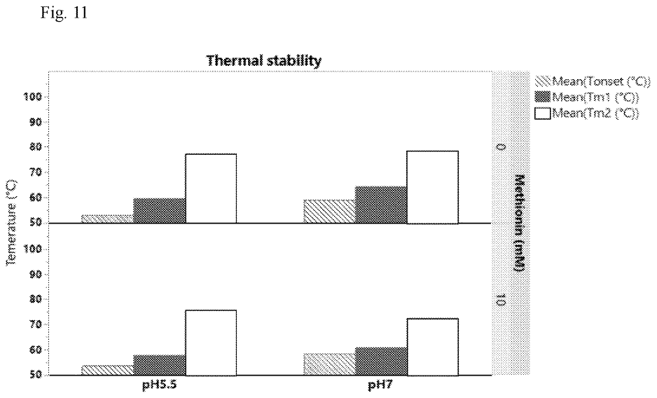

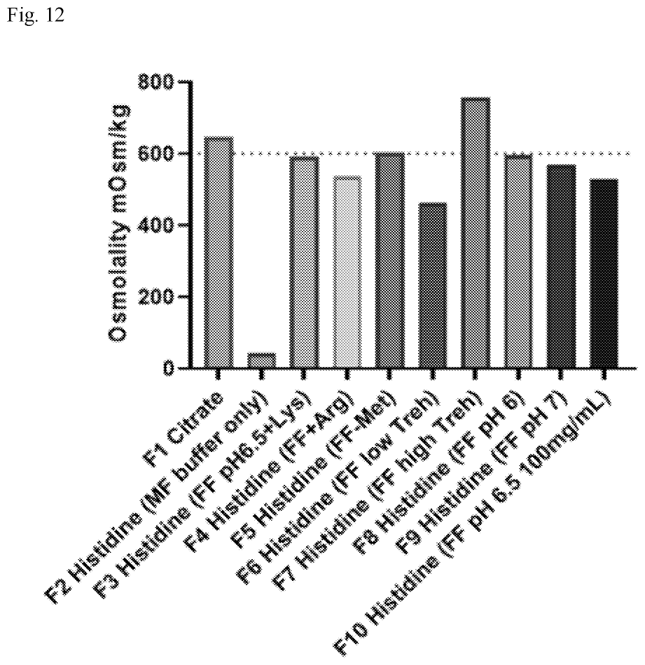

Non-limiting examples of substitutions that can be introduced to generate a suitable knob (e.g., in the part that binds to IL-18 or IL-13) include S354C and/or T366W (EU numbering). Non-limiting examples of substitutions that can be introduced to generate a suitable hole (e.g., in the part that binds to IL-13 or IL-18, depending on the type of substitution that was introduced in the other part) include Y349C, T366S, L368A, and/or Y407V (EU numbering). Additional knob-in-hole substitutions suitable for use in multispecific antibodies or antigen-binding fragments thereof of the present disclosure are described in WO2014/145806 (for example, FIG. 3, FIG. 4 and FIG. 12 of WO2014/145806), WO2014/110601, WO2016/086186, WO2016/086189, WO2016/086196, WO2016/182751 and WO 1996/027011. Another example of a K-i-H variant comprises a first constant chain comprising a L368D and a K370S mutation, paired with a second constant chain comprising a S364K and E357Q mutation (EU numbering).

Alternatively, heterodimerization may be enhanced by introduction of FAE substitutions, which are particularly suitable when Fab arm exchange is used to obtain the multispecific antibody or antigen-binding fragment thereof. The Fab arm exchange approach is described e.g. by Labrijn et al., Proc Natl Acad Sci USA. 2013 Mar. 26; 110 (13): 5145-50, and involves mixing of two separately expressed parental antibodies under controlled reducing conditions to separate the antibodies into half-antibodies and allow the recombining of half antibodies to form e.g., a bispecific antibody. In an embodiment, the FAE substitutions comprise a K409R substitution introduced to one CH3 domain paired with F405L substitution in the second CH3 domain, according to the EU numbering scheme, or vice versa. In some embodiments, additional substitutions may also, or alternatively, be introduced at positions selected from the group consisting of positions 366, 368, 370, 399, 405, 407, and 409 according to the EU numbering scheme.

The multispecific antibodies or antigen-binding fragments thereof described herein can be further modified to have an extended half-life in vivo. A variety of strategies known to the skilled person can be used to this end, e.g., chemical linkage to polyethylene glycol (PEG), reCODE PEG, antibody scaffold, polysialic acid (PSA), hydroxyethyl starch (HES), albumin-binding ligands, and carbohydrate shields; genetic fusion to proteins binding to serum proteins, such as albumin, IgG, FcRn, and transferring; coupling (genetically or chemically) to other binding moieties that bind to serum proteins such as nanobodies, Fabs, DARPins, avimers, affibodies, and anticalins; genetic fusion to rPEG, albumin, domain of albumin, albumin-binding proteins, and Fc, or by incorporation into nanocarriers or slow release formulations.

Multispecific antibodies or antigen-binding fragments thereof having an increased half-life in vivo can also be generated introducing one or more amino acid modifications (mutations) (i.e., substitutions, insertions or deletions) into an IgG constant domain, or FcRn binding fragment thereof (preferably a Fc polypeptide or fragment thereof). See, e.g., WO1998/23289; WO1997/34631; WO2013/209568, and U.S. Pat. No. 6,277,375. Half-life extension modifications that may be introduced to the Fc polypeptides of the multispecific preferably bispecific, antibodies or antigen-binding fragments thereof include the “LS” mutation (M428L, N434S, (EU numbering)) and the “YTE” mutation (M252Y, S254T, T256E (EU Numbering)), among which the “YTE” mutation (M252Y, S254T, T256E (EU Numbering)) is preferred.

Further, the multispecific antibodies or antigen-binding fragments thereof can be conjugated or fused to one or more human serum albumin (HSA) polypeptides, or a portion thereof. Albumin can be used to make the molecules more stable in vivo or have a longer half-life in vivo. Such techniques are known in the art, see, e.g., WO1993/15199, WO1993/15200, WO2001/77137, and EP413622. The use of N-terminal fragments of HSA for fusions to polypeptides has also been described (e.g. EP399666). Accordingly, genetically or chemically fusing or conjugating the molecules to albumin can stabilize or extend the shelf-life, and/or retain the molecule's activity for extended periods of time in solution, in vitro and/or in vivo. Additional methods pertaining to HSA fusions can be found, for example, in WO2001/077137 and WO2003/06007. In some embodiments, the expression of the fusion protein is performed in mammalian cell lines, for example, CHO cell lines.

In some cases, it can be beneficial to include one or more mutations to silence effector function, e.g., ADCC and/or CDC effector function within the Fc polypeptide of the multispecific, preferably bispecific, antibodies or antigen-binding fragments thereof, referred to herein as “silencing mutations”. Crosslinking of Fc regions via gamma receptors may induce antibody dependent cellular cytotoxicity (ADCC). Human Fc regions when complexed at the cell surface can also bind complement proteins and induce complement dependent cytotoxicity (CDC). Mutations to residues in Fc which reduce or abrogate these interactions may thus limit these effects and focus the impact of the antibodies or fragments thereof described herein upon target cells. More than one mutation, for example amino acid substitutions, can be combined. Non-limiting examples of combinations of amino acid substitutions for antibody effector silencing include the “DAPA” mutation (D265A, P329A in EU numbering) or the “LALA” mutation (L234A, L235A in EU numbering). Other exemplary silencing mutations include the “LALAGA” mutation (L234A, L235A, G237A in EU numbering), the “LALASKPA” mutation (L234A, L235A, S267K, P329A in EU numbering, e.g., as described in WO2022/097065), the “DAPASK” mutation (D265A, P329A, S267K in EU numbering), the “GADAPA” mutation (G237A, D265A, P329A in EU numbering), the “GADAPASK” mutation (G237A, D265A, P329A, S267K in EU numbering), the “LALAPG” mutation (L234A, L235A, P329G in EU numbering), and the “LALAPA” mutation (L234A, L235A, P329A in EU numbering). Further examples of silencing mutations are disclosed in WO2022/097065.

In some embodiments, one or more, e.g., all, of the heavy chain constant region domains of the multispecific, preferably bispecific, antibody or antigen-binding fragment thereof comprise an N279A mutation (according to EU numbering) (see, e.g., Tao M H, Morrison S L. Studies of aglycosylated chimeric mouse-human IgG. Role of carbohydrate in the structure and effector functions mediated by the human IgG constant region. J Immunol. 1989; 143 (8): 2595-601; Shields R L, Namenuk A K, Hong K, Meng Y G, Rae J, Briggs J, Xie D, Lai J, Stadlen A, Li B, Fox J A, Presta L G. High resolution mapping of the binding site on human IgG1 for Fc gamma RI, Fc gamma RII, Fc gamma RIII, and FcRn and design of IgG1 variants with improved binding to the Fc gamma R. J Biol Chem. 2001; 276 (9): 6591-604). Additional Fc mutations for providing silenced effector function are described in WO2014/145806. One example of a silent IgG1 antibody comprises a E233P, L234V, L235A, and S267K mutation, and a deletion of G236 (G236del) (EU numbering). Another example of a silent IgG1 antibody comprises a E233P, L234V, and L235A mutation, and a deletion of G236 (G236del) (EU numbering). Another example of a silent IgG1 antibody comprises a S267K mutation (EU numbering).

Accordingly, in some embodiments, the first and/or second Fc polypeptide comprise a variant of SEQ ID NO: 59 comprising one or more amino acid substitutions selected from the group consisting of:
a. one or more half-life extension substitutions, e.g., an M252Y, an S254T, and a T256E substitution (“YTE”), and;
b. one or more K-i-H substitutions, e.g., an S354C and/or a T366W substitution or a Y349C, a T366S, an L368A, and/or a Y407V substitution, according to EU numbering.

Preferably,
the first part that binds to IL-18 comprises a first Fc polypeptide comprising a variant of SEQ ID NO: 59 comprising:
   a) an M252Y, an S254T, and a T256E substitution (“YTE”), and;
   b) a Y349C, a T366S, an L368A, and a Y407V substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) an S354C and a T366W substitution,
      or
the first part that binds to IL-18 comprises a first Fc polypeptide comprising a variant of SEQ ID NO: 59 comprising:
   a) an M252Y, an S254T, and a T256E substitution (“YTE”), and;
   b) an S354C and a T366W substitution, and;
the second part that binds to IL-13 comprises a second Fc polypeptide comprising a variant of SEQ ID NO: 59 comprising:
   c) an M252Y, an S254T, and a T256E substitution (“YTE”), and;
   d) a Y349C, a T366S, an L368A, and a Y407V substitution, according to EU numbering.

In some embodiments, the first part that binds to IL-18 comprises a first Fc polypeptide comprising SEQ ID NO: 60 or SEQ ID NO: 61, e.g., SEQ ID NO: 60. In some embodiments, the second part that binds to IL-13 comprises a second Fc polypeptide comprising SEQ ID NO: 61 or SEQ ID NO: 60, e.g., SEQ ID NO: 61.

In some embodiments, the multispecific, preferably bispecific, antibody or antigen-binding fragment thereof comprises:
i) a first part that binds to IL-18 comprising a first heavy chain (HCl) comprising or consisting of SEQ ID NO: 42 or 57, or comprising or consisting of a sequence having at least 80%, at least 85%, at least 90%, at least 95%, at least 96%, at least 97%, at least 98%, or at least 99% sequence identity to SEQ ID NO: 42 or 57, and a first light chain (LC1) comprising or consisting of SEQ ID NO: 14, or comprising or consisting of a sequence having at least 80%, at least 85%, at least 90%, at least 95%, at least 96%, at least 97%, at least 98%, or at least 99% sequence identity to SEQ ID NO: 14, and;
ii) a second part that binds to IL-13 comprising a second heavy chain (HC2) comprising or consisting of SEQ ID NO: 56 or 58, or comprising or consisting of a sequence having at least 80%, at least 85%, at least 90%, at least 95%, at least 96%, at least 97%, at least 98%, or at least 99% sequence identity to SEQ ID NO: 56 or 58, and a second light chain (LC2) comprising or consisting of SEQ ID NO: 28, or comprising or consisting of a sequence having at least 80%, at least 85%, at least 90%, at least 95%, at least 96%, at least 97%, at least 98%, or at least 99% sequence identity to SEQ ID NO: 28.

In some embodiments, the multispecific, preferably bispecific, antibody or antigen-binding fragment thereof comprises:
i) a first part that binds to IL-18 comprising a first heavy chain (HCl) comprising or consisting of SEQ ID NO: 42, or comprising or consisting of a sequence having at least 80%, at least 85%, at least 90%, at least 95%, at least 96%, at least 97%, at least 98%, or at least 99% sequence identity to SEQ ID NO: 42, and a first light chain (LC1) comprising or consisting of SEQ ID NO: 14, or comprising or consisting of a sequence having at least 80%, at least 85%, at least 90%, at least 95%, at least 96%, at least 97%, at least 98%, or at least 99% sequence identity SEQ ID NO: 14, and;
ii) a second part that binds to IL-13 comprising a second heavy chain (HC2) comprising or consisting of SEQ ID NO: 56, or comprising or consisting of a sequence having at least 80%, at least 85%, at least 90%, at least 95%, at least 96%, at least 97%, at least 98%, or at least 99% sequence identity to SEQ ID NO: 56, and a second light chain (LC2) comprising or consisting of SEQ ID NO: 28, or comprising or consisting of a sequence having at least 80%, at least 85%, at least 90%, at least 95%, at least 96%, at least 97%, at least 98%, or at least 99% sequence identity to SEQ ID NO: 28.

In some embodiments, the multispecific, preferably bispecific, antibody or antigen-binding fragment thereof consists of:
i) a first part that binds to IL-18 consisting of a first heavy chain (HCl) consisting of SEQ ID NO: 42, or consisting of a sequence having at least 80%, at least 85%, at least 90%, at least 95%, at least 96%, at least 97%, at least 98%, or at least 99% sequence identity to SEQ ID NO: 42, and a first light chain (LC1) consisting of SEQ ID NO: 14, or consisting of a sequence having at least 80%, at least 85%, at least 90%, at least 95%, at least 96%, at least 97%, at least 98%, or at least 99% sequence identity SEQ ID NO: 14, and;
ii) a second part that binds to IL-13 consisting of a second heavy chain (HC2) consisting of SEQ ID NO: 56, or consisting of a sequence having at least 80%, at least 85%, at least 90%, at least 95%, at least 96%, at least 97%, at least 98%, or at least 99% sequence identity to SEQ ID NO: 56, and a second light chain (LC2) consisting of SEQ ID NO: 28, or consisting of a sequence having at least 80%, at least 85%, at least 90%, at least 95%, at least 96%, at least 97%, at least 98%, or at least 99% sequence identity to SEQ ID NO: 28.

In some embodiments, the multispecific, preferably bispecific, antibody or antigen-binding fragment thereof comprises:
i) a first part that binds to IL-18 comprising a first heavy chain (HCl) comprising or consisting of SEQ ID NO: 57, or comprising or consisting of a sequence having at least 80%, at least 85%, at least 90%, at least 95%, at least 96%, at least 97%, at least 98%, or at least 99% sequence identity to SEQ ID NO: 57, and a first light chain (LC1) comprising or consisting of SEQ ID NO: 14, or comprising or consisting of a sequence having at least 80%, at least 85%, at least 90%, at least 95%, at least 96%, at least 97%, at least 98%, or at least 99% sequence identity to SEQ ID NO: 14, and;
ii) a second part that binds to IL-13 comprising a second heavy chain (HC2) comprising or consisting of SEQ ID NO: 58, or comprising or consisting of a sequence having at least 80%, at least 85%, at least 90%, at least 95%, at least 96%, at least 97%, at least 98%, or at least 99% sequence identity to SEQ ID NO: 58, and a second light chain (LC2) comprising or consisting of SEQ ID NO: 28, or comprising or consisting of a sequence having at least 80%, at least 85%, at least 90%, at least 95%, at least 96%, at least 97%, at least 98%, or at least 99% sequence identity to SEQ ID NO: 28.

In some embodiments, the multispecific, preferably bispecific, antibody or antigen-binding fragment thereof consists of:
i) a first part that binds to IL-18 consisting of a first heavy chain (HCl) consisting of SEQ ID NO: 57, or consisting of a sequence having at least 80%, at least 85%, at least 90%, at least 95%, at least 96%, at least 97%, at least 98%, or at least 99% sequence identity to SEQ ID NO: 57, and a first light chain (LC1) consisting of SEQ ID NO: 14, or consisting of a sequence having at least 80%, at least 85%, at least 90%, at least 95%, at least 96%, at least 97%, at least 98%, or at least 99% sequence identity to SEQ ID NO: 14, and;
ii) a second part that binds to IL-13 consisting of a second heavy chain (HC2) consisting of SEQ ID NO: 58, or consisting of a sequence having at least 80%, at least 85%, at least 90%, at least 95%, at least 96%, at least 97%, at least 98%, or at least 99% sequence identity to SEQ ID NO: 58, and a second light chain (LC2) consisting of SEQ ID NO: 28, or consisting of a sequence having at least 80%, at least 85%, at least 90%, at least 95%, at least 96%, at least 97%, at least 98%, or at least 99% sequence identity to SEQ ID NO: 28.

TABLE 1
Non-limiting exemplary multispecific antibody sequences.
IL-18 binding part

| | | |
|---|---|---|
| SEQ ID NO: 1 (Combined) | LCDR1 | SGSSSNIGNHYVN |
| SEQ ID NO: 2 (Combined) | LCDR2 | RNNHRPS |
| SEQ ID NO: 3 (Combined) | LCDR3 | QSWDYSGFSTV |
| SEQ ID NO: 4 (Kabat) | LCDR1 | SGSSSNIGNHYVN |
| SEQ ID NO: 5 (Kabat) | LCDR2 | RNNHRPS |
| SEQ ID NO: 6 (Kabat) | LCDR3 | QSWDYSGFSTV |
| SEQ ID NO: 7 (Chothia) | LCDR1 | SSSNIGNHY |
| | LCDR2 | RNN |
| SEQ ID NO: 9 (Chothia) | LCDR3 | WDYSGFST |
| SEQ ID NO: 10 (IMGT) | LCDRI | SSNIGNHY |
| | LCDR2 | RNN |
| SEQ ID NO: 12 (IMGT) | LCDR3 | QSWDYSGFSTV |
| SEQ ID NO: 13 | VL | DIVLTQPPSVSGAPGQRVTISCSGSSSNIGNHYVN |
| | | WYQQLPGTAPKLLIYRNNHRPSGVPDRFSGSKS |
| | | GTSASLAITGLQSEDEADYYCQSWDYSGFSTVF |
| | | GGGTKLTVL |
| SEQ ID NO: 14 | Light Chain 1 | DIVLTQPPSVSGAPGQRVTISCSGSSSNIGNHYVN |
| | | WYQQLPGTAPKLLIYRNNHRPSGVPDRFSGSKS |
| | | GTSASLAITGLQSEDEADYYCQSWDYSGFSTVF |
| | | GGGTKLTVLGQPKAAPSVTLFPPSSEELQANKAT |
| | | LVCLISDFYPGAVTVAWKADSSPVKAGVETTTP |
| | | SKQSNNKYAASSYLSLTPEQWKSHRSYSCQVTH |
| | | EGSTVEKTVAPTECS |
| SEQ ID NO: 29 | HCDR1 | GGTFKSYAIS |
| (Combined) | | |
| SEQ ID NO: 30 | HCDR2 | NIIPMTGQTYYAQKFQG |
| (Combined) | | |
| SEQ ID NO: 31 | HCDR3 | AAYHPLVFDN |
| (Combined) | | |
| SEQ ID NO: 32 (Kabat) | HCDR1 | SYAIS |
| SEQ ID NO: 33 (Kabat) | HCDR2 | NIIPMTGQTYYAQKFQG |
| SEQ ID NO: 34 (Kabat) | HCDR3 | AAYHPLVFDN |
| SEQ ID NO: 35 (Chothia) | HCDR1 | GGTFKSY |
| SEQ ID NO: 36 (Chothia) | HCDR2 | IPMTGQ |
| SEQ ID NO: 37 (Chothia) | HCDR3 | AAYHPLVFDN |
| SEQ ID NO: 38 (IMGT) | HCDR1 | GGTFKSYA |
| SEQ ID NO: 39 (IMGT) | HCDR2 | IIPMTGQT |
| SEQ ID NO: 40 (IMGT) | HCDR3 | ARAAYHPLVFDN |
| SEQ ID NO: 41 | VH | EVQLVQSGAEVKKPGSSVKVSCKASGGTFKSYA |
| | | ISWVRQAPGQGLEWMGNIIPMTGQTYYAQKFQ |
| | | GRVTITADESTSTAYMELSSLRSEDTAVYYCARA |
| | | AYHPLVFDNWGQGTLVTVSS |
| SEQ ID NO: 42 | Heavy Chain 1 | EVQLVQSGAEVKKPGSSVKVSCKASGGTFKSYA |
| | | ISWVRQAPGQGLEWMGNIIPMTGQTYYAQKFQ |
| | | GRVTITADESTSTAYMELSSLRSEDTAVYYCARA |
| | | AYHPLVFDNWGQGTLVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQT |
| | | YICNVNHKPSNTKVDKRVEPKSCDKTHTCPPCP |
| | | APELLGGPSVFLFPPKPKDTLYITREPEVTCVVVD |
| | | VSHEDPEVKFNWYVDGVEVHNAKTKPREEQYN |
| | | STYRVVSVLTVLHQDWLNGKEYKCKVSNKALP |
| | | APIEKTISKAKGQPREPQVCTLPPSREEMTKNQV |
| | | SLSCAVKGFYPSDIAVEWESNGQPENNYKTTPPV |
| | | LDSDGSFFLVSKLTVDKSRWQQGNVFSCSVMHE |
| | | ALHNHYTQKSLSLSPGK |
| SEQ ID NO: 57 | Heavy chain 1 | EVQLVQSGAEVKKPGSSVKVSCKASGGTFKSYA |
| | | ISWVRQAPGQGLEWMGNIIPMTGQTYYAQKFQ |
| | | GRVTITADESTSTAYMELSSLRSEDTAVYYCARA |
| | | AYHPLVFDNWGQGTLVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQT |
| | | YICNVNHKPSNTKVDKRVEPKSCDKTHTCPPCP |
| | | APELLGGPSVFLFPPKPKDTLYITREPEVTCVVVD |
| | | VSHEDPEVKFNWYVDGVEVHNAKTKPREEQYN |
| | | STYRVVSVLTVLHQDWLNGKEYKCKVSNKALP |
| | | APIEKTISKAKGQPREPQVYTLPPCREEMTKNQV |
| | | SLWCLVKGFYPSDIAVEWESNGQPENNYKTTPP |
| | | VLDSDGSFFLYSKLTVDKSRWQQGNVFSCSVMH |
| | | EALHNHYTQKSLSLSPGK |

IL-13 binding part

| | | |
|---|---|---|
| SEQ ID NO: 15 | LCDR1 | RASKSVDSYGNSFMH |
| (Combined) | | |
| SEQ ID NO: 16 | LCDR2 | LASNLES |
| (Combined) | | |
| SEQ ID NO: 17 | LCDR3 | QQNNEDPRT |
| (Combined) | | |
| SEQ ID NO: 18 (Kabat) | LCDR1 | RASKSVDSYGNSFMH |
| SEQ ID NO: 19 (Kabat) | LCDR2 | LASNLES |
| SEQ ID NO: 20 (Kabat) | LCDR3 | QQNNEDPRT |
| SEQ ID NO: 21 (Chothia) | LCDR1 | SKSVDSYGNSF |
| | LCDR2 | LAS |
| SEQ ID NO: 23 (Chothia) | LCDR3 | NNEDPR |
| SEQ ID NO: 24 (IMGT) | LCDR1 | KSVDSYGNSF |
| | LCDR2 | LAS |
| SEQ ID NO: 26 (IMGT) | LCDR3 | QQNNEDPRT |
| SEQ ID NO: 27 | VL | DIVMTQSPDSLSVSLGERATINCRASKSVDSYGN |
| | | SFMHWYQQKPGQPPKLLIYLASNLESGVPDRFS |
| | | GSGSGTDFTLTISSLQAEDVAVYYCQQNNEDPRT |
| | | FGGGTKVEIK |
| SEQ ID NO: 28 | Light Chain 2 | DIVMTQSPDSLSVSLGERATINCRASKSVDSYGN |
| | | SFMHWYQQKPGQPPKLLIYLASNLESGVPDRFS |
| | | GSGSGTDFTLTISSLQAEDVAVYYCQQNNEDPRT |
| | | FGGGTKVEIKRTVAAPSVFIFPPSDEQLKSGTASV |
| | | VCLLNNFYPREAKVQWKVDNALQSGNSQESVT |
| | | EQDSKDSTYSLSSTLTLSKADYEKHKVYACEVT |
| | | HQGLSSPVTKSFNRGEC |
| SEQ ID NO: 43 | HCDR1 | GFSLSAYSVN |
| (Combined) | | |
| SEQ ID NO: 44 | HCDR2 | MIWGDGKIVYNSALKS |
| (Combined) | | |
| SEQ ID NO: 45 | HCDR3 | DGYYPYAMDN |
| (Combined) | | |
| SEQ ID NO: 46 (Kabat) | HCDR1 | AYSVN |
| SEQ ID NO: 47 (Kabat) | HCDR2 | MIWGDGKIVYNSALKS |
| SEQ ID NO: 48 (Kabat) | HCDR3 | DGYYPYAMDN |
| SEQ ID NO: 49 (Chothia) | HCDR1 | GFSLSAY |
| SEQ ID NO: 50 (Chothia) | HCDR2 | WGDGK |
| SEQ ID NO: 51 (Chothia) | HCDR3 | DGYYPYAMDN |
| SEQ ID NO: 52 (IMGT) | HCDR1 | GFSLSAYS |
| SEQ ID NO: 53 (IMGT) | HCDR2 | IWGDGKI |
| SEQ ID NO: 54 (IMGT) | HCDR3 | AGDGYYPYAMDN |
| SEQ ID NO: 55 | VH | QVTLRESGPALVKPTQTLTLTCTVSGFSLSAYSV |
| | | NWIRQPPGKALEWLAMIWGDGKIVYNSALKSRL |
| | | TISKDTSKNQVVLTMTNMDPVDTATYYCAGDG |
| | | YYPYAMDNWGQGSLVTVSS |
| SEQ ID NO: 56 | Heavy Chain 2 | QVTLRESGPALVKPTQTLTLTCTVSGFSLSAYSV |
| | | NWIRQPPGKALEWLAMIWGDGKIVYNSALKSRL |
| | | TISKDTSKNQVVLTMTNMDPVDTATYYCAGDG |
| | | YYPYAMDNWGQGSLVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQTYI |
| | | CNVNHKPSNTKVDKRVEPKSCDKTHTCPPCPAP |
| | | ELLGGPSVFLFPPKPKDTLYITREPEVTCVVVDVS |
| | | HEDPEVKFNWYVDGVEVHNAKTKPREEQYNST |
| | | YRVVSVLTVLHQDWLNGKEYKCKVSNKALPAP |
| | | IEKTISKAKGQPREPQVYTLPPCREEMTKNQVSL |
| | | WCLVKGFYPSDIAVEWESNGQPENNYKTTPPVL |
| | | DSDGSFFLYSKLTVDKSRWQQGNVFSCSVMHEA |
| | | LHNHYTQKSLSLSPGK |
| SEQ ID NO: 58 | Heavy chain 2 | QVTLRESGPALVKPTQTLTLTCTVSGFSLSAYSV |
| | | NWIRQPPGKALEWLAMIWGDGKIVYNSALKSRL |
| | | TISKDTSKNQVVLTMTNMDPVDTATYYCAGDG |
| | | YYPYAMDNWGQGSLVTVSSASTKGPSVFPLAPS |
| | | SKSTSGGTAALGCLVKDYFPEPVTVSWNSGALT |
| | | SGVHTFPAVLQSSGLYSLSSVVTVPSSSLGTQTYI |
| | | CNVNHKPSNTKVDKRVEPKSCDKTHTCPPCPAP |
| | | ELLGGPSVFLFPPKPKDTLYITREPEVTCVVVDVS |
| | | HEDPEVKFNWYVDGVEVHNAKTKPREEQYNST |
| | | YRVVSVLTVLHQDWLNGKEYKCKVSNKALPAP |
| | | IEKTISKAKGQPREPQVCTLPPSREEMTKNQVSLS |
| | | CAVKGFYPSDIAVEWESNGQPENNYKTTPPVLD |
| | | SDGSFFLVSKLTVDKSRWQQGNVFSCSVMHEAL |
| | | HNHYTQKSLSLSPGK |
| SEQ ID NO: 59 | Fc | DKTHTCPPCPAPELLGGPSVFLFPPKPKDTLMISR |
| | | TPEVTCVVVDVSHEDPEVKFNWYVDGVEVHNA |
| | | KTKPREEQYNSTYRVVSVLTVLHQDWLNGKEY |
| | | KCKVSNKALPAPIEKTISKAKGQPREPQVYTLPP |
| | | SREEMTKNQVSLTCLVKGFYPSDIAVEWESNGQ |
| | | PENNYKTTPPVLDSDGSFFLYSKLTVDKSRWQQ |
| | | GNVFSCSVMHEALHNHYTQKSLSLSPGK |
| SEQ ID NO: 60 | Fc-hole | DKTHTCPPCPAPELLGGPSVFLFPPKPKDTLYITR |
| | | EPEVTCVVVDVSHEDPEVKFNWYVDGVEVHNA |
| | | KTKPREEQYNSTYRVVSVLTVLHQDWLNGKEY |
| | | KCKVSNKALPAPIEKTISKAKGQPREPQVCTLPPS |
| | | REEMTKNQVSLSCAVKGFYPSDIAVEWESNGQP |
| | | ENNYKTTPPVLDSDGSFFLVSKLTVDKSRWQQG |
| | | NVFSCSVMHEALHNHYTQKSLSLSPGK |
| SEQ ID NO: 61 | Fc-key | DKTHTCPPCPAPELLGGPSVFLFPPKPKDTLYITR |
| | | EPEVTCVVVDVSHEDPEVKFNWYVDGVEVHNA |
| | | KTKPREEQYNSTYRVVSVLTVLHQDWLNGKEY |
| | | KCKVSNKALPAPIEKTISKAKGQPREPQVYTLPP |
| | | CREEMTKNQVSLWCLVKGFYPSDIAVEWESNG |
| | | QPENNYKTTPPVLDSDGSFFLYSKLTVDKSRWQ |
| | | QGNVFSCSVMHEALHNHYTQKSLSLSPGK |

Amino acid sequence variants of the antibodies described herein can e.g., be prepared by introducing appropriate nucleotide changes into their encoding nucleic acid molecules, or by synthesis of the desired variants. Such variants include, for example, variants arising from deletions, insertions, or substitutions, of residues within the amino acid sequences of present antibodies. Any suitable combination of deletions, insertions, and/or substitutions can be made to arrive at a variant construct, provided that the final construct possesses the desired antigen-binding characteristics. The amino acid changes also may alter post-translational processes of the antibodies, such as changing the number or position of glycosylation sites. The present application further includes variants of the antibodies described herein and/or fragments thereof having amino acid conservative modifications in variable regions and/or constant regions.

In some embodiments, a multispecific, preferably bispecific, antibody described herein or antigen-binding fragment thereof comprises an IL-18 binding part comprising an HCDR1, HCDR2, HCDR3, LCDR1, LCDR2, and/or LCDR3 region with a sequence shown in Table 1, or an HCDR1, HCDR2, HCDR3, LCDR1, LCDR2, and/or LCDR3 region having at least 80%, at least 85%, at least 90%, at least 95%, at least 96%, at least 97%, at least 98%, or at least 99% sequence identity with the sequence of an HCDR1, HCDR2, HCDR3, LCDR1, LCDR2, and/or LCDR3 region shown in Table 1. In some embodiments, a multispecific, preferably bispecific, antibody or antigen-binding fragment thereof described herein comprises an IL-18 binding part comprising an HCDR1, HCDR2, HCDR3, LCDR1, LCDR2, and/or LCDR3 region having 1, 2, or 3 substitutions, deletions, or insertions relative to the sequence of an HCDR1, HCDR2, HCDR3, LCDR1, LCDR2, and/or LCDR3 region shown in Table 1.

In some embodiments, a multispecific, preferably bispecific, antibody described herein or antigen-binding fragment thereof comprises an IL-13 binding part comprising a HCDR1, HCDR2, HCDR3, LCDR1, LCDR2, and/or LCDR3 region with a sequence shown in Table 1, or an HCDR1, HCDR2, HCDR3, LCDR1, LCDR2, and/or LCDR3 region having at least 80%, at least 85%, at least 90%, at least 95%, at least 96%, at least 97%, at least 98%, or at least 99% sequence identity with the sequence of an HCDR1, HCDR2, HCDR3, LCDR1, LCDR2, and/or LCDR3 region shown in Table 1. In some embodiments, a multispecific, preferably bispecific, antibody or antigen-binding fragment thereof described herein comprises an IL-13 binding part comprising an HCDR1, HCDR2, HCDR3, LCDR1, LCDR2, and/or LCDR3 region having 1, 2, or 3 substitutions, deletions, or insertions relative to the sequence of an HCDR1, HCDR2, HCDR3, LCDR1, LCDR2, and/or LCDR3 region shown in Table 1.

In some embodiments, a multispecific, preferably bispecific, antibody or antigen-binding fragment thereof described herein comprises an IL-18 binding part comprising a VH and/or VL region with a sequence shown in Table 1, or a VH and/or VL region having at least 80%, at least 85%, at least 90%, at least 95%, at least 96%, at least 97%, at least 98%, or at least 99% sequence identity with the sequence of a VH and/or VL region shown in Table 1. In some embodiments, a multispecific, preferably bispecific, antibody or antigen-binding fragment thereof described herein comprises an IL-18 binding part comprising a VL and/or VH region having 1, 2, or 3 substitutions, deletions, or insertions relative to the sequence of a VL and/or VH region shown in Table 1.

In some embodiments, a multispecific, preferably bispecific, antibody or antigen-binding fragment thereof described herein comprises an IL-13 binding part comprising a VH and/or VL region with a sequence shown in Table 1, or a VH and/or VL region having at least 80%, at least 85%, at least 90%, at least 95%, at least 96%, at least 97%, at least 98%, or at least 99% sequence identity with the sequence of a VH and/or VL region shown in Table 1. In some embodiments, a multispecific, preferably bispecific, antibody or antigen-binding fragment thereof described herein comprises an IL-13 binding part comprising a VL and/or VH region having 1, 2, or 3 substitutions, deletions, or insertions relative to the sequence of a VL and/or VH region shown in Table 1.

In some embodiments, a multispecific, preferably bispecific, antibody or antigen-binding fragment thereof described herein comprises an IL-18 binding part comprising a heavy chain and/or light chain with a sequence shown in Table 1, or a heavy chain and/or light chain having at least 80%, at least 85%, at least 90%, at least 95%, at least 96%, at least 97%, at least 98%, or at least 99% sequence identity with the sequence of a heavy chain and/or light chain shown in Table 1. In some embodiments, a multispecific, preferably bispecific, antibody or antigen-binding fragment thereof described herein comprises an IL-18 binding part comprising a heavy chain and/or light chain having 1, 2, or 3 substitutions, deletions, or insertions relative to the sequence of a heavy chain and/or light chain shown in Table 1.

In some embodiments, a multispecific, preferably bispecific, antibody or antigen-binding fragment thereof described herein comprises an IL-13 binding part comprising a heavy chain and/or light chain with a sequence shown in Table 1, or a heavy chain and/or light chain having at least 80%, at least 85%, at least 90%, at least 95%, at least 96%, at least 97%, at least 98%, or at least 99% sequence identity with the sequence of a heavy chain and/or light chain shown in Table 1. In some embodiments, a multispecific, preferably bispecific, antibody or antigen-binding fragment thereof described herein comprises an IL-13 binding part comprising a heavy chain and/or light chain having 1, 2, or 3 substitutions, deletions, or insertions relative to the sequence of a heavy chain and/or light chain shown in Table 1.

In some embodiments, the substitutions are conservative substitutions, as described earlier herein.

In some embodiments, a multispecific, preferably bispecific, antibody or antigen-binding fragment thereof comprises a first part that binds to IL-18 and a second part that binds to IL-13 as described in WO2023/209568.

### Pharmaceutical Compositions

The pharmaceutical, preferably liquid (e.g., aqueous), compositions described herein are based, at least in part, in the discovery that the multispecific, preferably bispecific, antibodies and antigen-binding fragments thereof described herein can be formulated at high concentrations while maintaining low viscosity and excellent properties, such as one or more properties selected from the group consisting of conformational (thermal) stability, colloidal stability, physical stability, chemical stability, and biological activity. In particular, liquid (e.g., aqueous) compositions described herein can be associated with one or more of decreased aggregation, deamidation, and/or oxidation at high multispecific antibody concentrations relative to pharmaceutical compositions previously available in the art.

The term “high concentration” refers to a pharmaceutical composition comprising about 15 mg/mL of the multispecific antibody (or antigen-binding fragment thereof) or more (about ≥15 mg/mL), e.g., about 20 mg/mL or more (about ≥20 mg/mL), about 25 mg/mL or more (about ≥25 mg/mL), about 30 mg/mL or more (about ≥30 mg/mL), about 35 mg/mL or more (about ≥35 mg/mL), about 40 mg/mL or more (about ≥40 mg/mL), about 45 mg/mL or more (about ≥45 mg/mL), about 50 mg/mL or more (about ≥50 mg/mL), about 55 mg/mL or more (about ≥55 mg/mL), about 60 mg/mL or more (about ≥60 mg/mL), about 65 mg/mL or more (about ≥65 mg/mL), about 70 mg/mL or more (about ≥70 mg/mL), about 75 mg/mL or more (about ≥75 mg/mL), about 80 mg/mL or more (about ≥80 mg/mL), about 85 mg/mL or more (about ≥85 mg/mL), about 90 mg/mL or more (about ≥90 mg/mL), about 95 mg/mL or more (about ≥95 mg/mL), about 100 mg/mL or more (about ≥100 mg/mL), about 105 mg/mL or more (about ≥105 mg/mL), about 110 mg/mL or more (about ≥110 mg/mL), about 115 mg/mL or more (about ≥115 mg/mL), about 120 mg/mL or more (about ≥120 mg/mL), about 125 mg/mL or more (about ≥125 mg/mL), about 130 mg/mL or more (about ≥130 mg/mL), about 135 mg/mL or more (about ≥135 mg/mL), about 140 mg/mL or more (about ≥140 mg/mL), about 145 mg/mL or more (about ≥145 mg/mL), about 150 mg/mL or more (about ≥150 mg/mL), about 155 mg/mL or more (about ≥155 mg/mL), about 160 mg/mL or more (about ≥160 mg/mL), about 165 mg/mL or more (about ≥165 mg/mL), about 170 mg/mL or more (about ≥170 mg/mL), about 175 mg/mL or more (about ≥175 mg/mL), about 180 mg/mL or more (about ≥180 mg/mL), about 185 mg/mL or more (about ≥185 mg/mL), about 190 mg/mL or more (about ≥190 mg/mL), about 195 mg/mL or more (about ≥195 mg/mL), or about 200 mg/mL or more (about ≥200 mg/mL). Accordingly, in some embodiments, the pharmaceutical composition comprises a high concentration of the multispecific antibody or antigen-binding fragment thereof.

In some embodiments, the pharmaceutical composition comprises about 50 mg/mL or more (about ≥50 mg/mL) of the multispecific antibody or antigen-binding fragment thereof. In some embodiments, the pharmaceutical composition comprises about 75 mg/mL (about ≥75 mg/mL) of the multispecific antibody or antigen-binding fragment thereof. In some embodiments, the pharmaceutical composition comprises about 100 mg/mL or more (about ≥100 mg/ml) of the multispecific antibody or antigen-binding fragment thereof. In some embodiments, the pharmaceutical composition comprises about 125 mg/mL (about ≥125 mg/ml) of the multispecific antibody or antigen-binding fragment thereof. In some embodiments, a preferred concentration of the multispecific antibody or antigen-binding fragment thereof is about 150 mg/mL or more (about >150 mg/mL), e.g., about 150 mg/mL or 150 mg/mL.

In some embodiments, the pharmaceutical composition comprises from about 15 mg/ml to about 200 mg/mL of the multispecific antibody or antigen-binding fragment thereof, e.g., from about to 25 mg/mL to about 190 mg/mL, from about 35 mg/mL to about 180 mg/mL, from about 50 mg/mL to about 175 mg/mL, from about 50 mg/mL to about 150 mg/mL, from about 75 mg/mL to about 150 mg/mL, from about 100 mg/mL to about 150 mg/mL, from about 125 mg/mL to about 150 mg/mL, preferably from about 50 mg/mL, from about 75 mg/mL, or from about 100 mg/mL to about 150 mg/mL, e.g., from about 50 mg/mL to about 150 mg/mL (e.g., about 150 mg/mL or 150 mg/mL). In some embodiments, the pharmaceutical composition comprises from about 50 mg/mL to about 200 mg/mL of the multispecific antibody or antigen-binding fragment thereof, e.g., from about 50 mg/ml to about 175 mg/ml, from about 75 mg/mL to about 175 mg/mL, or from about 100 mg/mL to about 175 mg/mL, e.g., from about 50 mg/mL to about 150 mg/ml (e.g., about 150 mg/mL or 150 mg/mL).

In some embodiments, the pharmaceutical composition comprises from about 20 mg/ml to about 190 mg/mL of the multispecific antibody or antigen-binding fragment thereof, e.g., from about 30 mg/mL to about 180 mg/mL, from about 40 mg/mL to about 170 mg/mL, from about 50 mg/mL to about 160 mg/mL, from about 60 mg/mL to about 150 mg/mL, from about 70 mg/mL to about 150 mg/mL, from about 80 mg/mL to about 150 mg/mL, or from about 90 mg/mL to about 150 mg/mL.

In some embodiments, the pharmaceutical composition comprises about 15 mg/mL (e.g., 15 mg/mL), about 20 mg/mL (e.g., 20 mg/mL), about 25 mg/mL (e.g., 25 mg/mL), about 30 mg/mL (e.g., 30 mg/mL), about 35 mg/mL (e.g., 35 mg/mL), about 40 mg/mL (e.g., 40 mg/mL), about 45 mg/mL (e.g., 45 mg/mL), about 50 mg/mL (e.g., 50 mg/mL), about 55 mg/mL (e.g., 55 mg/mL), about 60 mg/mL (e.g., 60 mg/mL), about 65 mg/mL (e.g., 65 mg/mL), about 70 mg/mL (e.g., 70 mg/mL), about 75 mg/mL (e.g., 75 mg/mL), about 80 mg/mL (e.g., 80 mg/mL), about 85 mg/mL (e.g., 85 mg/mL), about 90 mg/mL (e.g., 90 mg/mL), about 95 mg/ml (e.g., 95 mg/mL), about 100 mg/mL (e.g., 100 mg/mL), about 105 mg/mL (e.g., 105 mg/mL), about 110 mg/ml (e.g., 110 mg/mL), about 115 mg/mL (e.g., 115 mg/mL), about 120 mg/mL (e.g., 120 mg/mL), about 125 mg/mL (e.g., 125 mg/mL), about 130 mg/mL (e.g., 130 mg/mL), about 135 mg/mL (e.g., 135 mg/mL), about 140 mg/mL (e.g., 140 mg/mL), about 145 mg/mL (e.g., 145 mg/mL), about 150 mg/mL (e.g., 150 mg/mL), about 155 mg/mL (e.g., 155 mg/mL), about 160 mg/mL (e.g., 160 mg/mL), about 165 mg/mL (e.g., 165 mg/mL), about 170 mg/mL (e.g., 170 mg/mL), about 175 mg/mL (e.g., 175 mg/mL), about 180 mg/mL (e.g., 180 mg/mL), about 185 mg/ml (e.g., 185 mg/mL), about 190 mg/mL (e.g., 190 mg/mL), about 195 mg/mL (e.g., 195 mg/mL), about 200 mg/mL (e.g., 200 mg/mL), or more, of the multispecific antibody or antigen-binding fragment thereof.

Antibodies tend to form viscous solutions at high concentration because of their macromolecular nature and potential for intermolecular interactions. In particular, the present inventors have found that multispecific antibodies described herein, when not comprised in a pharmaceutical composition described herein, have a tendency towards developing high viscosity at high concentrations. Highly viscous formulations are difficult to manufacture, draw into a syringe and to inject. Further, the use of force in manipulating the viscous formulations can lead to excessive frothing, which can lead to denaturation and inactivation of the multispecific antibodies.

The pharmaceutical, preferably liquid (e.g., aqueous), compositions described herein advantageously maintain low viscosity at high multispecific antibody (or antigen-binding fragment thereof) concentrations. “Low viscosity” as used herein refers to the pharmaceutical composition having a viscosity of about 20 cP (or mPa*s) or lower (about ≤20 cP (or mPa*s)), about 19 cP (or mPa*s) or lower (about ≤19 cP (or mPa*s)), about 18 cP (or mPa*s) or lower (about ≤18 cP (or mPa*s)), about 17 cP (or mPa*s) or lower (about ≤17 cP (or mPa*s)), about 16 cP (or mPa*s) or lower (about ≤16 cP (or mPa*s)), or about 15 cP (or mPa*s) or lower (about ≤15 cP (or mPa*s)), preferably about 15 cP (or mPa*s) or lower, preferably measured at room temperature, e.g., from about 20° C. to about 25° C., e.g., about 25° C. (e.g., 25° C.).

Viscosity of pharmaceutical compositions may be measured using techniques and instruments available to the skilled person, e.g., using a rheometer and applying a shear-rate of from about 200 s⁻¹ to about 2000 s⁻¹, e.g., about 1000 s⁻¹. Viscosity may e.g., be measured by applying a constant shear rate, or alternatively shear-rate sweeps may be applied, in which shear rate is ramped up to the desired shear rate followed by ramping down (more than 1 ramp-up/ramp-down cycles can be applied). Alternatively, viscosity may be measured using dynamic light scattering (e.g., by rearranging the Stokes-Einstein equation (He et al. (2010), Anal Biochem 399 (1): 141-143)). Examples of viscosity measurements are provided in the experimental section later herein.

The pharmaceutical compositions described herein typically comprise at least one pharmaceutically acceptable ingredient or excipient. The phrase “pharmaceutically acceptable” means approved by a regulatory agency of a federal or a state government, or listed in the U.S. Pharmacopeia or other generally recognized pharmacopeia for use in animals, and more particularly, in humans. Pharmaceutically acceptable ingredients include, without limitation, pharmaceutically acceptable carriers, fillers, stabilizers, viscosity reducing agents, preservatives, solubilizers, vehicles, diluents, and/or excipients.

The pharmaceutical compositions described herein are thus suitable for parenteral administration, e.g., intravenous or subcutaneous administration, preferably for subcutaneous administration. A preferred pharmaceutical composition is a liquid pharmaceutical composition, e.g., an aqueous pharmaceutical composition, e.g., a solution or suspension for injection (e.g., subcutaneous injection). A liquid composition should typically be sterile and fluid.

The pharmaceutical, preferably liquid (e.g., aqueous), compositions described herein may advantageously maintain sufficiently low osmolality to avoid injection pain, e.g., subcutaneous injection pain. In some embodiments, the pharmaceutical composition has an osmolality of ≤800 mOsm/kg, e.g., ≤700 mOsm/kg or ≤600 mOsm/kg, preferably ≤600 mOsm/kg, e.g., as measured with freezing point depression (FPD). Suitable instruments for osmolality measurements are commercially available, and e.g., include the Advanced Instruments Model 2020 osmometer (Advanced Instruments, MA, USA).

Non-limiting examples of administration routes include intravenous, intramuscular, intradermal, intraperitoneal, subcutaneous, spinal, and other parenteral routes of administration, for example by injection or infusion. Parenteral administration can represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a pharmaceutical composition of the present disclosure can be administered via a non-parenteral route, such as a topical, epidermal or mucosal route of administration, for example, intranasally, orally, vaginally, rectally, sublingually, or topically.

The pharmaceutical, preferably liquid (e.g., aqueous), compositions described herein may in some embodiments comprise, in addition to the multispecific antibody or antigen-binding fragment thereof, one or more pharmaceutically acceptable ingredients selected from the group consisting of: (i) a buffering agent, (ii) one or more stabilizers (e.g., one, two, three, or more stabilizers), (iii) a viscosity reducing agent, (iv) an anti-oxidant, and v) a surfactant. It is understood that a certain ingredient may exert one or more than one effects on the properties of the pharmaceutical composition. As a non-limiting example, a buffering agent may also have a stabilizing effect. As another non-limiting example, a viscosity reducing agent may also have a stabilizing effect, e.g., a free amino acid may act as a stabilizer and a viscosity reducing agent. As another non-limiting example, a viscosity reducing agent may also have a buffering effect. As another non-limiting example, a stabilizer may also have an anti-oxidant effect. Thus, references to excipients herein as stabilizers, viscosity reducing agents, anti-oxidants, and the like, should not be construed as limiting in terms of their function. More information on pharmaceutical acceptable ingredients or excipients that may be comprised by the pharmaceutical compositions described herein is provided in the following paragraphs.

In some embodiments, the pharmaceutical, preferably liquid (e.g., aqueous), compositions are stable pharmaceutical compositions. In some embodiments, the pharmaceutical, preferably liquid (e.g., aqueous), compositions are stable over a selected time period (e.g., over at least 4 weeks, at least 3 months, or at least 6 months (or longer)) upon storage.

Stability may e.g., refer to conformational (thermal) stability, colloidal stability, physical stability, chemical stability, and/or biological activity of the multispecific antibody or antigen-binding fragment thereof, and a “stable” composition refers to the multispecific antibody or antigen-binding fragment thereof retaining its stability (one or more of its conformational (thermal) stability, colloidal stability, physical stability, chemical stability, biological activity) upon storage (relative to an unformulated antibody or antigen-binding fragment thereof). Stability may be e.g., assessed at a selected temperature over a selected time period, e.g., over at least 4 weeks, at least 3 months, at least 6 months, or longer. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993), and further examples are provided in the experimental section later herein.

An antibody may e.g., be said to retain its physical stability in a pharmaceutical composition if it shows no significant increase in aggregation, precipitation and/or denaturation upon e.g., visual examination of color and/or clarity (turbidity), or as measured by UV light scattering, size exclusion chromatography (SEC), dynamic light scattering (DLS), or another suitable method. The pharmaceutical compositions described herein in particular can enhance the physical stability of the multispecific antibody or antigen-binding fragment thereof under stress conditions, e.g., against shear stress and/or temperature stress (thermal stability).

An antibody may e.g., be said to retain its chemical stability in a pharmaceutical composition if it shows no significant chemical alteration. Chemical stability can be assessed by detecting and quantifying chemically altered forms of the protein. Degradation processes that often alter the protein chemical structure include hydrolysis or clipping (e.g., measured by size exclusion chromatography (SEC), SDS-PAGE, or another suitable method), oxidation (e.g., measured by peptide mapping in conjunction with mass spectroscopy or MALDI/TOF/MS, multiple-attribute-methodology mass spectrometry (MAM-MS), capillary zone electrophoresis (CZE), or another suitable method), deamidation (e.g., measured by cation-exchange chromatography (CEX), multiple-attribute-methodology mass spectrometry (MAM-MS), capillary zone electrophoresis (CZE), capillary isoelectric focusing, peptide mapping, isoaspartic acid measurement, or another suitable method), racemization, pyroglutamate formation, and isomerization (e.g., evaluated by measuring isoaspartic acid content, peptide mapping, or another suitable method).

An antibody may e.g., be said to retain its biological activity in a pharmaceutical composition, if the biological activity of the antibody at a given time is within a predetermined range (e.g., 70%-130%, 80%-120%, or 90%-120%) of the biological activity exhibited at the time the pharmaceutical composition was prepared. The biological activity of an antibody can be determined, for example, by an antigen binding ELISA assay, potency assay, cysteamine-CEX derivatization, or another suitable method.

In some embodiments, the pharmaceutical composition is stable at a refrigerated temperature, e.g., from about 2° C. to about 8° C., e.g., about 5° C., for at least 1 month, at least 2 months, at least 3 months, at least 4 months, at least 5 months, at least 6 months, or longer, e.g., at least 6 months. In some embodiments, the pharmaceutical composition is stable at room temperature, e.g., from about 20° C. to about 25° C., e.g., about 25° C., for at least 1 week, at least 2 weeks, at least 3 weeks, at least 4 weeks, at least 1 month, at least 2 months, or at least 3 months, e.g., at least 3 months. In some embodiments, the pharmaceutical composition is stable at temperature stress, e.g., about 40° C., for at least 1 week, at least 2 weeks, or at least 3 weeks, or at least 4 weeks, e.g., at least 4 weeks. In some embodiments, stability may be measured by e.g., additionally (or alternatively) applying freeze-thaw stress (e.g., for 1, 2, 3, 4, 5, or more freeze-thaw cycles) and/or shear/shaking stress, as e.g., described later herein.

In some embodiments, purity, as measured by SEC, of the multispecific antibody in a stable pharmaceutical composition is decreased (relative to a reference starting timepoint) by no more than about 10%, e.g., no more than about 9.5%, than about 9%, than about 8.5%, than about 8%, than about 7.5%, than about 7%, than about 6.5%, than about 6%, than about 5.5%, than about 5%, than about 4.5%, than about 4%, than about 3.5%, than about 3%, than about 2.5%, than about 2%, than about 1.5%, than about 1%, or than about 0.5%, e.g., no more than about 1% or than about 0.5%, upon storage for at least 3 or at least 6 months at a refrigerated temperature, e.g., from about 2° C. to about 8° C., e.g., about 5° C.

In some embodiments, purity, as measured by SEC, of the multispecific antibody in a stable pharmaceutical composition is decreased (relative to a reference starting timepoint) by no more than about 10%, e.g., no more than about 9.5%, than about 9%, than about 8.5%, than about 8%, than about 7.5%, than about 7%, than about 6.5%, than about 6%, than about 5.5%, than about 5%, than about 4.5%, than about 4%, than about 3.5%, than about 3%, than about 2.5%, or than about 2%, e.g., no more than about 2%, upon storage for at least 1 or at least 3 months at room temperature, e.g., from about 20° C. to about 25° C., e.g., about 25° C.

In some embodiments, purity, as measured by SEC, of the multispecific antibody in a stable pharmaceutical composition is decreased (relative to a reference starting timepoint) by no more than about 10%, e.g., no more than about 9.5%, than about 9%, than about 8.5%, than about 8%, than about 7.5%, than about 7%, than about 6.5%, than about 6%, than about 5.5%, than about 5%, than about 4.5%, or than about 4%, e.g., no more than about 4%, e.g., upon storage for at least 2 weeks or at least 4 weeks at a temperature of about 40° C.

Purity as measured by SEC refers to the percentage of monomer multispecific antibody as measured by SEC. SEC is used to separate monomeric antibodies from aggregates according to their size under non-denaturing conditions. The sum of peaks eluting prior the main peak are typically reported as percentage of aggregation products (AP-SEC), the sum of peaks eluting after the main peak as percentage of degradation products (DP-SEC). A further example is provided in the experimental section later herein.

In some embodiments, the sum of basic variants of the multispecific antibody in a stable pharmaceutical composition is increased (relative to a reference starting timepoint) by no more than about 10%, e.g., no more than about 9.5%, than about 9%, than about 8.5%, than about 8%, than about 7.5%, than about 7%, than about 6.5%, than about 6%, than about 5.5%, or than about 5%, e.g., no more than about 5%, upon storage for at least 3 or at least 6 months at a refrigerated temperature, e.g., from about 2° C. to about 8° C., e.g., about 5° C., preferably as measured by capillary zone electrophoresis (CZE).

In some embodiments, the sum of basic variants of the multispecific antibody in a stable pharmaceutical composition is increased (relative to a reference starting timepoint) by no more than about 10% upon storage for at least 2 weeks, at least 4 weeks, or at least 3 months at room temperature, e.g., from about 20° C. to about 25° C., e.g., about 25° C., preferably as measured capillary zone electrophoresis (CZE).

In some embodiments, the sum of basic variants of the multispecific antibody in a stable pharmaceutical composition is increased (relative to a reference starting timepoint) by no more than about 10% upon storage for at least 2 weeks at a temperature of about 40° C., preferably as measured capillary zone electrophoresis (CZE).

In some embodiments, the sum of acidic variants of the multispecific antibody in a stable pharmaceutical composition is increased (relative to a reference starting timepoint) by no more than about 10%, e.g., no more than about 9.5%, than about 9%, than about 8.5%, than about 8%, than about 7.5%, than about 7%, than about 6.5%, than about 6%, than about 5.5%, than about 5%, than about 4.5%, than about 4%, e.g., no more than about 5% or than about 4%, upon storage for at least 3 or at least 6 months at a refrigerated temperature, e.g., from about 2° C. to about 8° C., e.g., about 5° C., preferably as measured by capillary zone electrophoresis (CZE).

In some embodiments, the sum of acidic variants of the multispecific antibody in a stable pharmaceutical composition is increased (relative to a reference starting timepoint) by no more than about 10% upon storage for at least 2 weeks, at least 4 weeks, or at least 3 months at room temperature, e.g., from about 20° C. to about 25° C., e.g., about 25° C., preferably as measured capillary zone electrophoresis (CZE).

In some embodiments, the sum of acidic variants of the multispecific antibody in a stable pharmaceutical composition is increased (relative to a reference starting timepoint) by no more than about 10% upon storage for at least 2 weeks at a temperature of about 40° C., preferably as measured capillary zone electrophoresis (CZE). In some embodiments, the sum of acidic variants of the multispecific antibody in a stable pharmaceutical composition is increased (relative to a reference starting timepoint) by no more than about 20%, e.g., no more than about 19% or than about 18%, upon storage for at least 4 weeks at a temperature of about 40° C., preferably as measured capillary zone electrophoresis (CZE).

The pharmaceutical compositions described herein may advantageously protect the multispecific antibody or antigen-binding fragment thereof against oxidation upon storage, even under temperature stress conditions e.g., about 40° C. Stability under oxidation stress may be assessed using any available method, e.g., forced oxidation with hydrogen peroxide (H₂O₂) may be applied, followed by MAM-MS to measure the increase in oxidized amino acids.

In some embodiments, oxidation of the multispecific antibody or antigen-binding fragment thereof is decreased relative to oxidation displayed by a reference composition (e.g., a reference pharmaceutical composition not comprising an ingredient described herein (e.g., methionine)) upon storage, e.g., for at least 4 weeks at a temperature of about 40° C.

In some embodiments wherein the first part that binds to IL-18 comprises a first variable light region (VL1) comprising SEQ ID NO: 13 and the second part that binds to IL-13 comprises a second variable light region (VL2) comprising SEQ ID NO: 27, oxidation of the M37 residue in SEQ ID NO: 27 (corresponding to a residue in the LCDR1 of VL2; numbering starting from D1 of SEQ ID NO: 27) is decreased relative to oxidation displayed by a reference composition (e.g., a reference pharmaceutical composition not comprising an ingredient described herein (e.g., methionine)) upon storage, e.g., for at least 4 weeks at a temperature of about 40° C. In some embodiments wherein the first part that binds to IL-18 comprises a first light chain (LC1) comprising SEQ ID NO: 14 and the second part that binds to IL-13 comprises a second light chain (LC2) comprising SEQ ID NO: 28, oxidation of the M37 residue in SEQ ID NO: 28 (corresponding to a residue in the LCDR1 of LC2; numbering starting from D1 of SEQ ID NO: 28) is decreased relative to oxidation displayed by a reference composition (e.g., a reference pharmaceutical composition not comprising an ingredient described herein (e.g., methionine)) upon storage, e.g., for at least 4 weeks at a temperature of about 40° C.

The pharmaceutical compositions described herein may advantageously protect the multispecific antibody or antigen-binding fragment thereof against deamidation upon storage, even under temperature stress conditions e.g., about 40° C. Deamidation may be assessed using any available method, e.g., MAM-MS may be used to measure asparagine (Asn (N)) conversion to aspartic acid (Asp (D)) or iso-aspartic acid (iso-Asp (iso-D)).

In some embodiments, deamidation of the multispecific antibody or antigen-binding fragment thereof is decreased relative to deamidation displayed by a reference composition (e.g., a reference pharmaceutical composition not comprising an ingredient described herein) upon storage, e.g., for at least 4 weeks at a temperature of about 40° C.

In some embodiments wherein the first part that binds to IL-18 comprises a first variable light region (VL1) comprising SEQ ID NO: 13 and the second part that binds to IL-13 comprises a second variable light region (VL2) comprising SEQ ID NO: 27, deamidation of the N28 and/or N31 residue in SEQ ID NO: 13 (corresponding to a residue in the LCDR1 of VL1; numbering starting from D1 of SEQ ID NO: 13) is decreased relative to deamidation displayed by a reference composition (e.g., a reference pharmaceutical composition not comprising an ingredient described herein) upon storage, e.g., for at least 4 weeks at a temperature of about 40° C. In some embodiments wherein the first part that binds to IL-18 comprises a first light chain (LC1) comprising SEQ ID NO: 14 and the second part that binds to IL-13 comprises a second light chain (LC2) comprising SEQ ID NO: 28, deamidation of the N28 and/or N31 residue in SEQ ID NO: 14 (corresponding to a residue in the LCDR1 of LC1; numbering starting from D1 of SEQ ID NO: 14) is decreased relative to deamidation displayed by a reference composition (e.g., a reference pharmaceutical composition not comprising an ingredient described herein) upon storage, e.g., for at least 4 weeks at a temperature of about 40° C.

In some embodiments wherein the first part that binds to IL-18 comprises a first heavy chain (HCl) comprising SEQ ID NO: 42 or SEQ ID NO: 57 and the second part that binds to IL-13 comprises a second heavy chain (HC2) comprising, respectively, SEQ ID NO: 56 or SEQ ID NO: 58, deamidation of the N386 and/or N391 residue in SEQ ID NO: 42 or SEQ ID NO: 57 (numbering starting from E1 of SEQ ID NO: 42 or SEQ ID NO: 57) and/or deamidation of the N385 and/or N390 residue in SEQ ID NO: 56 or SEQ ID NO: 58 (numbering starting from Q1 of SEQ ID NO: 56 or SEQ ID NO: 58) is decreased relative to deamidation displayed by a reference composition (e.g., a reference pharmaceutical composition not comprising an ingredient described herein) upon storage, e.g., for at least 4 weeks at a temperature of about 40° C.

The pharmaceutical compositions described herein may advantageously protect the multispecific antibody or antigen-binding fragment thereof against isomerization upon storage, even under temperature stress conditions e.g., about 40° C. Isomerization may be assessed using any available method, e.g., MAM-MS may be used to measure aspartic acid (Asp (D)) conversion to iso-aspartic acid (iso-Asp (iso-D)) and/or aspartic acid (Asp (D)) conversion to succinimide (intermediate).

In some embodiments, isomerization of the multispecific antibody or antigen-binding fragment thereof is decreased relative to isomerization displayed by a reference composition (e.g., a reference pharmaceutical composition not comprising an ingredient described herein) upon storage, e.g., for at least 4 weeks at a temperature of about 40° C.

In some embodiments wherein the first part that binds to IL-18 comprises a first variable heavy region (VH1) comprising SEQ ID NO: 41 and the second part that binds to IL-13 comprises a second variable heavy region (VH2) comprising SEQ ID NO: 55, isomerization of an aspartic acid residue selected from the group consisting of D54, D86, D89, D98, D106 in SEQ ID NO: 55 (numbering starting from Q1 of SEQ ID NO: 55), and a combination thereof, is decreased relative to isomerization displayed by a reference composition (e.g., a reference pharmaceutical composition not comprising an ingredient described herein) upon storage, e.g., for at least 4 weeks at a temperature of about 40° C. D54 corresponds to a residue in HCDR2 of SEQ ID NO: 55; D98 and D106 correspond to residues in HCDR3 of SEQ ID NO: 55.

In some embodiments wherein the first part that binds to IL-18 comprises a first heavy chain (HCl) comprising SEQ ID NO: 42 or SEQ ID NO: 57 and the second part that binds to IL-13 comprises a second heavy chain (HC2) comprising, respectively, SEQ ID NO: 56 or SEQ ID NO: 58, isomerization of an aspartic acid residue selected from the group consisting of D54, D86, D89, D98, D106 in SEQ ID NO: 56 or SEQ ID NO: 58 (numbering starting from Q1 of SEQ ID NO: 56 or SEQ ID NO: 58), and a combination thereof, is decreased relative to isomerization displayed by a reference composition (e.g., a reference pharmaceutical composition not comprising an ingredient described herein) upon storage, e.g., for at least 4 weeks at a temperature of about 40° C.

The pharmaceutical compositions described herein may advantageously protect the multispecific antibody or antigen-binding fragment thereof against pyroglutamate formation upon storage, even under temperature stress conditions e.g., about 40° C. Pyroglutamate formation may be assessed using any available method, e.g., MAM-MS may be used to measure glutamic acid (Glu (E)) conversion to pyroglutamic acid (pyro-Glu).

In some embodiments, pyroglutamate formation in the multispecific antibody or antigen-binding fragment thereof is decreased relative to pyroglutamate formation displayed by a reference composition (e.g., a reference pharmaceutical composition not comprising an ingredient described herein) upon storage, e.g., for at least 4 weeks at a temperature of about 40° C.

In some embodiments wherein the first part that binds to IL-18 comprises a first variable heavy region (VH1) comprising SEQ ID NO: 41 and the second part that binds to IL-13 comprises a second variable heavy region (VH2) comprising SEQ ID NO: 55, conversion of the E1 residue in SEQ ID NO: 41 to pyroglutamate is decreased relative to the one displayed by a reference composition (e.g., a reference pharmaceutical composition not comprising an ingredient described herein) upon storage, e.g., for at least 4 weeks at a temperature of about 40° C.

In some embodiments wherein the first part that binds to IL-18 comprises a first heavy chain (HCl) comprising SEQ ID NO: 42 or SEQ ID NO: 57 and the second part that binds to IL-13 comprises a second heavy chain (HC2) comprising, respectively, SEQ ID NO: 56 or SEQ ID NO: 58, conversion of the E1 residue in SEQ ID NO: 42 or SEQ ID NO: 57 to pyroglutamate is decreased relative to the one displayed by a reference composition (e.g., a reference pharmaceutical composition not comprising an ingredient described herein) upon storage, e.g., for at least 4 weeks at a temperature of about 40° C.

### Buffering Agents and pH

In some embodiments, the pharmaceutical, preferably liquid (e.g., aqueous), composition described herein comprises a buffering agent (buffer).

The term “buffering agent” or “buffer” or “pH adjusting agent” refers to a buffered solution that can resist changes in pH by the action of its components (typically acid-base conjugates). Such agents are typically included in a pharmaceutical composition to “set” its pH to a certain value and/or to provide pH control.

In some embodiments, the pharmaceutical composition has a pH of from about 5 to about 8, e.g., from about 5.5 to about 7.4. In some embodiments, the pharmaceutical composition has a pH of from about 5.6 to about 7.3, from about 5.7 to about 7.2, from about 5.8 to about 7.1, from about 5.9 to about 7, from about 6 to about 6.9, from about 6.1 to about 6.8, from about 6.2 to about 6.7, from about 6.3 to about 6.6, or from about 6.4 to about 6.5. In some embodiments, the pharmaceutical composition has a pH of from about 6 to about 7.

In some embodiments, the pharmaceutical composition has a pH of about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, or about 8. In preferred embodiments, the pharmaceutical composition has a pH of about 6.5, e.g., a pH of 6.5. In some embodiments, the pharmaceutical composition has a pH of 6.5±0.5.

In some embodiments, a pH value referred to herein corresponds to the pH value of the pharmaceutical composition at room temperature, e.g., from about 20° C. to about 25° C., e.g., about 25° C.

In some embodiments, the pharmaceutical composition comprises about 5 mM or more (about ≥5 mM) of the buffering agent, e.g., about 10 mM or more (about ≥10 mM), about 15 mM or more (about ≥15 mM), about 20 mM or more (about ≥20 mM), about 25 mM or more (about ≥25 mM), about 30 mM or more (about ≥30 mM), about 35 mM or more (about ≥35 mM), about 40 mM or more (about ≥40 mM), about 45 mM or more (about ≥45 mM), or about 50 mM or more (about ≥50 mM).

In some embodiments, the pharmaceutical composition comprises from about 1 mM to about 500 mM of the buffering agent. In some embodiments, the pharmaceutical composition comprises from about 5 mM to about 100 mM of the buffering agent.

In some embodiments, the pharmaceutical composition comprises from about 5 mM to about 50 mM of the buffering agent, e.g., from about 10 mM to about 40 mM, from about 15 mM to about 30 mM, or from about 20 mM to about 25 mM. In some embodiments, the pharmaceutical composition comprises from 10 mM to about 30 mM, to about 25 mM, or to about 20 mM of the buffering agent. In some embodiments, the pharmaceutical composition comprises from about 5 mM or from about 10 mM to about 20 mM of the buffering agent. In some embodiments, the pharmaceutical composition comprises from about 10 mM to about 25 mM of the buffering agent.

In some embodiments, the pharmaceutical composition comprises about 5 mM (e.g., 5 mM), about 6 mM (e.g., 6 mM), about 7 mM (e.g., 7 mM), about 8 mM (e.g., 8 mM), about 9 mM (e.g., 9 mM), about 10 mM (e.g., 10 mM), about 11 mM (e.g., 11 mM), about 12 mM (e.g., 12 mM), about 13 mM (e.g., 13 mM), about 14 mM (e.g., 14 mM), about 15 mM (e.g., 15 mM), about 16 mM (e.g., 16 mM), about 17 mM (e.g., 17 mM), about 18 mM (e.g., 18 mM), about 19 mM (e.g., 19 mM), about 20 mM (e.g., 20 mM), about 21 mM (e.g., 21 mM), about 22 mM (e.g., 22 mM), about 23 mM (e.g., 23 mM), about 24 mM (e.g., 24 mM), about 25 mM (e.g., 25 mM), about 26 mM (e.g., 26 mM), about 27 mM (e.g., 27 mM), about 28 mM (e.g., 28 mM), about 29 mM (e.g., 29 mM), about 30 mM (e.g., 30 mM), about 31 mM (e.g., 31 mM), about 32 mM (e.g., 32 mM), about 33 mM (e.g., 33 mM), about 34 mM (e.g., 34 mM), about 35 mM (e.g., 35 mM), about 36 mM (e.g., 36 mM), about 37 mM (e.g., 37 mM), about 38 mM (e.g., 38 mM), about 39 mM (e.g., 39 mM), about 40 mM (e.g., 40 mM), about 41 mM (e.g., 41 mM), about 42 mM (e.g., 42 mM), about 43 mM (e.g., 43 mM), about 44 mM (e.g., 44 mM), about 45 mM (e.g., 45 mM), about 46 mM (e.g., 46 mM), about 47 mM (e.g., 47 mM), about 48 mM (e.g., 48 mM), about 49 mM (e.g., 49 mM), about 50 mM (e.g., 50 mM), or more, of the buffering agent. In preferred embodiments, the pharmaceutical composition comprises about 20 mM of the buffering agent, e.g., 20 mM of the buffering agent.

Non-limiting examples of suitable buffering agents include organic acid buffers, citrate buffers, ascorbate buffers, gluconate buffers, carbonate buffers, tartarate buffers, succinate buffers, acetate buffers, phthalate buffers; Tris buffers, HCl buffers, and phosphate buffers. A buffering agent may be an amino-acid based buffering agent, e.g., an arginine buffer, a glycine buffer, or a histidine buffer. Other non-limiting example of suitable buffering agents include histidine acetate buffers, arginine acetate buffers, sodium acetate (Na-Acetate) buffers, histidine succinate, arginine succinate, sodium succinate, gluconate buffers, sodium citrate (Na-Citrate) buffers, sodium phosphate (Na-Phosphate) buffers, Tris-HCl buffers, and L-His/L-His HCl buffers.

Accordingly, in some embodiments, the buffering agent is selected from the group consisting of an acetate buffer, a citrate buffer, a phosphate buffer, a Tris buffer, a histidine buffer, and a combination thereof. Among buffering agents, histidine buffers (e.g., L-histidine buffers) are preferred. A “histidine buffer” is a buffering agent comprising the amino acid histidine (e.g., L-histidine) or a pharmaceutically acceptable salt thereof. Non-limiting examples of histidine buffers include histidine chloride, histidine acetate, histidine phosphate, and histidine sulfate buffers.

An exemplary histidine buffer that is particularly suitable is an L-His/L-His HCl buffer. In some embodiments, the histidine buffer is prepared by dissolving L-histidine and L-histidine hydrochloride in a defined amount and ratio to achieve the desired pH. In some embodiments, the histidine buffer is prepared by titrating L-histidine with (diluted) hydrochloric acid.

In some embodiments, the pharmaceutical composition comprises (in addition to the multispecific, preferably bispecific, antibody or antigen-binding fragment thereof described herein) one or more pharmaceutically acceptable ingredients selected from the group consisting of:
(i) a buffering agent, preferably a histidine buffer (e.g., an L-His/L-His HCl buffer);
(ii) a sugar stabilizer;
(iii) a viscosity reducing agent;
(iv) an anti-oxidant, and;
(v) a surfactant.

In some embodiments, the pharmaceutical composition comprises about 5 mM or more (about ≥5 mM) of a histidine buffer (e.g., L-histidine buffer, e.g., L-His/L-His HCl buffer), e.g., about 10 mM or more (about ≥10 mM), about 15 mM or more (about ≥15 mM), about 20 mM or more (about ≥20 mM), about 25 mM or more (about ≥25 mM), about 30 mM or more (about 30 mM), about 35 mM or more (about ≥35 mM), about 40 mM or more (about ≥40 mM), about 45 mM or more (about ≥45 mM), or about 50 mM or more (about ≥50 mM).

In some embodiments, the pharmaceutical composition comprises from about 1 mM to about 500 mM of a histidine buffer (e.g., L-histidine buffer, e.g., L-His/L-His HCl buffer). In some embodiments, the pharmaceutical composition comprises from about 5 mM to about 100 mM of a histidine buffer (e.g., L-histidine buffer, e.g., L-His/L-His HCl buffer).

In some embodiments, the pharmaceutical composition comprises from about 5 mM to about 50 mM of a histidine buffer (e.g., L-histidine buffer, e.g., L-His/L-His HCl buffer), e.g., from about 10 mM to about 40 mM, from about 15 mM to about 30 mM, or from about 20 mM to about 25 mM. In some embodiments, the pharmaceutical composition comprises from 10 mM to about 30 mM, to about 25 mM, or to about 20 mM of a histidine buffer (e.g., L-histidine buffer, e.g., L-His/L-His HCl buffer). In some embodiments, the pharmaceutical composition comprises from about 5 mM or from about 10 mM to about 20 mM of a histidine buffer (e.g., L-histidine buffer, e.g., L-His/L-His HCl buffer). In some embodiments, the pharmaceutical composition comprises from about 10 mM to about 25 mM of a histidine buffer (e.g., L-histidine buffer, e.g., L-His/L-His HCl buffer).

In some embodiments, the pharmaceutical composition comprises about 5 mM (e.g., 5 mM), about 6 mM (e.g., 6 mM), about 7 mM (e.g., 7 mM), about 8 mM (e.g., 8 mM), about 9 mM (e.g., 9 mM), about 10 mM (e.g., 10 mM), about 11 mM (e.g., 11 mM), about 12 mM (e.g., 12 mM), about 13 mM (e.g., 13 mM), about 14 mM (e.g., 14 mM), about 15 mM (e.g., 15 mM), about 16 mM (e.g., 16 mM), about 17 mM (e.g., 17 mM), about 18 mM (e.g., 18 mM), about 19 mM (e.g., 19 mM), about 20 mM (e.g., 20 mM), about 21 mM (e.g., 21 mM), about 22 mM (e.g., 22 mM), about 23 mM (e.g., 23 mM), about 24 mM (e.g., 24 mM), about 25 mM (e.g., 25 mM), about 26 mM (e.g., 26 mM), about 27 mM (e.g., 27 mM), about 28 mM (e.g., 28 mM), about 29 mM (e.g., 29 mM), about 30 mM (e.g., 30 mM), about 31 mM (e.g., 31 mM), about 32 mM (e.g., 32 mM), about 33 mM (e.g., 33 mM), about 34 mM (e.g., 34 mM), about 35 mM (e.g., 35 mM), about 36 mM (e.g., 36 mM), about 37 mM (e.g., 37 mM), about 38 mM (e.g., 38 mM), about 39 mM (e.g., 39 mM), about 40 mM (e.g., 40 mM), about 41 mM (e.g., 41 mM), about 42 mM (e.g., 42 mM), about 43 mM (e.g., 43 mM), about 44 mM (e.g., 44 mM), about 45 mM (e.g., 45 mM), about 46 mM (e.g., 46 mM), about 47 mM (e.g., 47 mM), about 48 mM (e.g., 48 mM), about 49 mM (e.g., 49 mM), about 50 mM (e.g., 50 mM), or more, of a histidine buffer (e.g., L-histidine buffer, e.g., L-His/L-His HCl buffer). In preferred embodiments, the pharmaceutical composition comprises about 20 mM of a histidine buffer (e.g., L-histidine buffer, e.g., L-His/L-His HCl buffer, e.g., 20 mM.

In some embodiments, the pharmaceutical composition comprises a histidine buffer (e.g., L-histidine buffer, e.g., L-His/L-His HCl buffer) at concentration of from about 5 mM to about 50 mM, e.g., from about 10 mM to about 25 mM, preferably about 20 mM (e.g., 20 mM).

With respect pharmaceutical compositions comprising histidine buffers, it is understood that the concentration refers to the total (sum of) histidine concentration. It is understood that, in embodiments wherein the histidine buffer is an L-His/L-His HCl buffer, the ratio of L-His to L-His HCl will vary depending on the desired pH, e.g., from about 5.5 to about 7.4 or from about 6 to about 7 (e.g., about 6.5 or 6.5) as described earlier herein.

### Stabilizers

In preferred embodiments, the pharmaceutical, preferably liquid (e.g., aqueous), composition described herein comprises one or more stabilizers (e.g., one, two, three, or more stabilizers).

Stabilizers assist in e.g., preventing oxidation and/or aggregation of proteins in pharmaceutical compositions, particularly liquid (e.g., aqueous) pharmaceutical compositions, which otherwise have a shorter shelf life due to tendency of proteins to oxidize and/or aggregate while in liquid solutions. A stabilizer may be ionic or non-ionic (e.g., sugar stabilizers). Non-limiting examples of stabilizers that may be comprised by pharmaceutical compositions described herein include polyols, sugar stabilizers (including sugar alcohols) (e.g., glucose, fructose, sucrose, dextrose, lactose, maltose, maltitol, lactitol, trehalose, stachyose, mannose, sorbose, xylose, ribose, ribitol, myo-inisitol, galactose, galactitol, glycerol, inositol, cellobiose, raffinose, melezitose, glycerin, erythritol, arabitol, xylitol, sorbitol, mannitol), free amino acids (e.g., alanine, glycine, glutamine, asparagine, histidine, arginine, lysine, omithine, leucine, 2-phenylalanine, glutamic acid, threonine, methionine), polyethylene glycol, sulfur-containing agents (e.g., urea, glutathione, thioctic acid, sodium thioglycolate, thioglycerol, α-monothioglycerol, sodium thiosulfate), low molecule weight proteins (e.g., human serum albumin, bovine serum albumin, gelatin), hydrophilic polymers (e.g., polyvinylpyrrolidone), and polysaccharides (e.g., dextrin, dextran (e.g., dextran 40). Ionic stabilizers include salts, e.g., sodium chloride (NaCl), sodium succinate, sodium sulfate, potassium chloride, magnesium chloride (MgCl₂), magnesium sulfate, and calcium chloride. Other non-limiting examples of salts that may be comprised by pharmaceutical compositions described herein include metal salts (e.g., Ca²⁺, Mg²⁺, Zn²⁺, Fe²⁺, Fe³⁺, Cu²⁺, Sn²⁺, Sn⁴⁺, Al²⁺, Al³⁺), inorganic acid salts (e.g., hydrochloric acid salts, sulfuric acid salts, nitric acid salts, phosphoric acid salts), organic acid salts (e.g., saturated or unsaturated C2-9 carboxylic acid salts, acetic acid salts, propionic acid salts, butyric acid salts, valeric acid salts, caproic acid salts, enanthic acid salts, caprylic acid salts, pelargonic acid salts, acrylic acid salts, methacrylic acid salts, crotonic acid salts, isocrotonic acid salts, malonic acid salts, succinic acid salts, glutaric acid salts, adipic acid salts, pimelic acid salts, maleic acid salts, fumaric acid salts, citraconic acid salts, mesaconic acid salts, tricarballylic acid salts, 1,2,3-butanetricarboxylic acid salts, glycolic acid salts, lactic acid salts, glyceric acid salts, tartronic acid salts, malic acid salts, tartaric acid salts, citric acid salts, benzoic acid salts, and salicylic acid salts. More information on free amino acids is provided later herein.

In preferred embodiments, the pharmaceutical composition comprises a sugar stabilizer, e.g., a sugar stabilizer described earlier herein. A sugar stabilizer may a reducing or a non-reducing sugar. A sugar stabilizer may be a sugar alcohol. A sugar stabilizer may be a sugar acid (e.g., gluconate and salts thereof). Non-limiting examples of reducing sugar stabilizers include fructose, mannose, maltose, lactose, arabinose, xylose, ribose, rhamnose, galactose, dextrose, and glucose. Non-limiting examples of non-reducing sugar stabilizers include sucrose, trehalose, sorbose, melezitose, and raffinose. Non-limiting examples of sugar alcohols include mannitol, xylitol, erythritol, threitol, sorbitol, and glycerol.

Accordingly, in some embodiments, the sugar stabilizer is a reducing sugar, e.g., selected from the group consisting of fructose, mannose, maltose, lactose, arabinose, xylose, ribose, rhamnose, galactose, dextrose, and glucose. In some embodiments, the sugar stabilizer is a non-reducing sugar, e.g., selected from the group consisting of sucrose, trehalose, sorbose, melezitose, and raffinose.

Among sugar stabilizers, trehalose or sucrose is preferred, with trehalose being more preferred. In some embodiments, the trehalose is trehalose dihydrate.

In some embodiments, the pharmaceutical composition comprises (in addition to the multispecific, preferably bispecific, antibody or antigen-binding fragment thereof described herein) one or more pharmaceutically acceptable ingredients selected from the group consisting of:
(i) a buffering agent as described earlier herein, preferably a histidine buffer (e.g., L-His/L-His HCl);
(ii) a sugar stabilizer, preferably trehalose or sucrose (e.g., trehalose, e.g., trehalose dihydrate);
(iii) a viscosity reducing agent;
(iv) an anti-oxidant, and;
(v) a surfactant.

In some embodiments, the pharmaceutical composition comprises from about 5 mM to about 500 mM of the sugar stabilizer, preferably trehalose or sucrose (e.g., trehalose, e.g., trehalose dihydrate). In some embodiments, the pharmaceutical composition comprises from about 5 mM to about 300 mM of the sugar stabilizer, preferably trehalose or sucrose (e.g., trehalose, e.g., trehalose dihydrate).

In some embodiments, the pharmaceutical composition comprises about 50 mM or more (about ≥50 mM) of the stabilizer, e.g., sugar stabilizer, preferably trehalose or sucrose (e.g., trehalose, e.g., trehalose dihydrate), e.g., about 55 mM or more (about ≥55 mM), about 60 mM or more (about ≥60 mM), about 65 mM or more (about ≥65 mM), about 70 mM or more (about ≥70 mM), about 75 mM or more (about ≥75 mM), about 80 mM or more (about ≥80 mM), about 85 mM or more (about >85 mM), about 90 mM or more (about ≥90 mM), about 95 mM or more (about ≥95 mM), about 100 mM or more (about ≥100 mM), about 105 mM or more (about ≥105 mM), about 110 mM or more (about ≥110 mM), about 115 mM or more (about ≥115 mM), about 120 mM or more (about ≥120 mM), about 125 mM or more (about ≥125 mM), about 130 mM or more (about ≥130 mM), about 135 mM or more (about ≥135 mM), about 140 mM or more (about ≥140 mM), about 145 mg/mL or more (about ≥145 mM), about 150 mM or more (about ≥150 mM), about 155 mM or more (about ≥155 mM), about 160 mM or more (about ≥160 mM), about 165 mM or more (about ≥165 mM), about 170 mM or more (about ≥170 mM), about 175 mM or more (about ≥175 mM), about 180 mM or more (about ≥180 mM), about 185 mM or more (about ≥185 mM), about 190 mM or more (about ≥190 mM), about 195 mM or more (about ≥195 mM), about 200 mM or more (about ≥200 mM), about 205 mM or more (about ≥205 mM), about 210 mM or more (about ≥210 mM), about 215 mM or more (about ≥215 mM), or about 220 mM or more (about ≥220 mM).

In some embodiments, the pharmaceutical composition comprises from about 50 mM to about 220 mM of the stabilizer, e.g., sugar stabilizer, preferably trehalose or sucrose (e.g., trehalose, e.g., trehalose dihydrate), e.g., from about 60 mM to about 200 mM, from about 70 mM to about 180 mM, from about 80 mM to about 160 mM, from about 90 mM to about 150 mM, from about 100 mM to about 150 mM, from about 110 mM to about 140 mM, or from about 110 mM or from about 120 mM to about 130 mM.

In some embodiments, the pharmaceutical composition comprises a stabilizer, e.g., sugar stabilizer, preferably trehalose or sucrose (e.g., trehalose, e.g., trehalose dihydrate), at a concentration of from about 100 mM, from about 110 mM, or from about 120 mM to about 150 mM, e.g., about 120 mM (e.g., 120 mM). In some embodiments, the pharmaceutical composition comprises from about 100 mM to about 150 mM trehalose or sucrose, e.g., about 120 mM trehalose or sucrose (e.g., trehalose, e.g., trehalose dihydrate). In some embodiments, the pharmaceutical composition comprises from about 100 mM to about 150 mM trehalose, e.g., about 120 mM trehalose (e.g., 120 mM trehalose). In some embodiments, the pharmaceutical composition comprises from about 100 mM to about 150 mM trehalose dihydrate, e.g., about 120 mM trehalose dihydrate (e.g., 120 mM trehalose dihydrate).

In some embodiments, the pharmaceutical composition comprises about 50 mM (e.g., 50 mM), about 55 mM (e.g., 55 mM), about 60 mM (e.g., 60 mM), about 65 mM (e.g., 65 mM), about 70 mM (e.g., 70 mM), about 75 mM (e.g., 75 mM), about 80 mM (e.g., 80 mM), about 85 mM (e.g., 85 mM), about 90 mM (e.g., 90 mM), about 95 mM (e.g., 95 mM), about 100 mM (e.g., 100 mM), about 105 mM (e.g., 105 mM), about 110 mM (e.g., 110 mM), about 115 mM (e.g., 115 mM), about 120 mM (e.g., 120 mM), about 125 mM (e.g., 125 mM), about 130 mM (e.g., 130 mM), about 135 mM (e.g., 135 mM), about 140 mM (e.g., 140 mM), about 145 mM (e.g., 145 mM), about 150 mM (e.g., 150 mM), about 155 mM (e.g., 155 mM), about 160 mM (e.g., 160 mM), about 165 mM (e.g., 165 mM), about 170 mM (e.g., 170 mM), about 175 mM (e.g., 175 mM), about 180 mM (e.g., 180 mM), about 185 mM (e.g., 185 mM), about 190 mM (e.g., 190 mM), about 195 mM (e.g., 195 mM), about 200 mM (e.g., 200 mM), about 205 mM (e.g., 205 mM), about 210 mM (e.g., 210 mM), about 215 mM (e.g., 215 mM), or about 220 mM (e.g., 220 mM), of the stabilizer, e.g., sugar stabilizer, preferably trehalose or sucrose (e.g., trehalose, e.g., trehalose dihydrate). In some embodiments, the pharmaceutical composition comprises about 120 mM trehalose (e.g., 120 mM trehalose), e.g., trehalose dihydrate.

### Viscosity Reducing Agents

In preferred embodiments, the pharmaceutical, preferably liquid (e.g., aqueous), composition described herein comprises a viscosity reducing agent. A pharmaceutical composition comprising a viscosity reducing agent will have a decreased viscosity relative to pharmaceutical composition not comprising the viscosity reducing agent, e.g., viscosity may be decreased by at least 5%, at least 10%, or more. A viscosity reducing agent may exert more than one effects on the properties of the pharmaceutical composition. As a non-limiting example, a viscosity reducing agent may also act as a stabilizer and/or anti-oxidant and/or may provide a buffering effect.

In some embodiments, the pharmaceutical composition comprises about 2.5 mM or more (about ≥2.5 mM) of the viscosity reducing agent, e.g., about 5 mM or more, about 10 mM or more, about 15 mM or more, about 20 mM or more, about 25 mM or more, about 30 mM or more, about 35 mM or more, about 40 mM or more, about 45 mM or more, about 50 mM or more, about 55 mM or more, about 60 mM or more, about 65 mM or more, about 70 mM or more, about 75 mM or more, about 80 mM or more, about 85 mM or more, about 90 mM or more, about 95 mM or more, about 100 mM or more, about 105 mM or more, about 110 mM or more, about 115 mM or more, about 120 mM or more, about 125 mM or more, about 130 mM or more, about 135 mM or more, about 140 mM or more, about 145 mM or more, about 150 mM or more, about 155 mM or more, about 160 mM or more, about 165 mM or more, about 170 mM or more, about 175 mM or more, about 180 mM or more, about 185 mM or more, about 190 mM or more, about 195 mM or more, about 200 mM or more, about 205 mM or more, about 210 mM or more, about 215 mM or more, about 220 mM or more, about 225 mM or more, about 230 mM or more, about 235 mM or more, about 240 mM or more, about 245 mM or more, or about 250 mM or more.

In some embodiments, the pharmaceutical composition comprises from about 2.5 mM to about 500 mM of a viscosity reducing agent. In some embodiments, the pharmaceutical composition comprises from about 2.5 mM to about 300 mM of a viscosity reducing agent.

In some embodiments, the pharmaceutical composition comprises from about 100 mM to about 250 mM of viscosity reducing agent, e.g., from about 105 mM to about 225 mM, from about 110 mM to about 200 mM, or from about 115 mM, about 120 mM, about 125 mM to about 175 mM of the viscosity reducing agent. In some embodiments, the pharmaceutical composition comprises from about 100 mM to about 200 mM of the viscosity reducing agent. In some embodiments, the pharmaceutical composition comprises from about 125 mM to about 175 mM of the viscosity reducing agent.

In some embodiments, the pharmaceutical composition comprises about 2.5 mM (e.g., 2.5 mM), about 5 mM (e.g., 5 mM), about 7.5 mM (e.g., 7.5 mM), about 10 mM (e.g., 10 mM), about 12.5 mM (e.g., 12.5 mM), about 15 mM (e.g., 15 mM), about 17.5 mM (e.g., 17.5 mM), about 20 mM (e.g., 20 mM), about 22.5 mM (e.g., 22.5 mM), about 25 mM (e.g., 25 mM), about 27.5 mM (e.g., 27.5 mM), about 30 mM (e.g., 30 mM), about 32.5 mM (e.g., 32.5 mM), about 35 mM (e.g., 35 mM), about 37.5 mM (e.g., 37.5 mM), about 40 mM (e.g., 40 mM), about 42.5 mM (e.g., 42.5 mM), about 45 mM (e.g., 45 mM), about 47.5 mM (e.g., 47.5 mM), about 50 mM (e.g., 50 mM), about 52.5 mM (e.g., 52.5 mM), about 55 mM (e.g., 55 mM), about 57.5 mM (e.g., 57.5 mM), about 60 mM (e.g., 60 mM), about 62.5 mM (e.g., 62.5 mM), about 65 mM (e.g., 65 mM), about 67.5 mM (e.g., 67.5 mM), about 70 mM (e.g., 70 mM), about 72.5 mM (e.g., 72.5 mM), about 75 mM (e.g., 75 mM), about 77.5 mM (e.g., 77.5 mM), about 80 mM (e.g., 80 mM), about 82.5 mM (e.g., 82.5 mM), about 85 mM (e.g., 85 mM), about 87.5 mM (e.g., 87.5 mM), about 90 mM (e.g., 90 mM), about 92.5 mM (e.g., 92.5 mM), about 95 mM (e.g., 95 mM), about 97.5 mM (e.g., 97.5 mM), about 100 mM (e.g., 100 mM), about 102.5 mM (e.g., 102.5 mM), about 105 mM (e.g., 105 mM), about 107.5 mM (e.g., 107.5 mM), about 110 mM (e.g., 110 mM), about 112.5 mM (e.g., 112.5 mM), about 115 mM (e.g., 115 mM), about 117.5 mM (e.g., 117.5 mM), about 120 mM (e.g., 120 mM), about 122.5 mM (e.g., 122.5 mM), about 125 mM (e.g., 125 mM), about 127.5 mM (e.g., 127.5 mM), about 130 mM (e.g., 130 mM), about 132.5 mM (e.g., 132.5 mM), about 135 mM (e.g., 135 mM), about 137.5 mM (e.g., 137.5 mM), about 140 mM (e.g., 140 mM), about 142.5 mM (e.g., 142.5 mM), about 145 mM (e.g., 145 mM), about 147.5 mM (e.g., 147.5 mM), about 150 mM (e.g., 150 mM), about 152.5 mM (e.g., 152.5 mM), about 155 mM (e.g., 155 mM), about 157.5 mM (e.g., 157.5 mM), about 160 mM (e.g., 160 mM), about 162.5 mM (e.g., 162.5 mM), about 165 mM (e.g., 165 mM), about 167.5 mM (e.g., 167.5 mM), about 170 mM (e.g., 170 mM), about 172.5 mM (e.g., 172.5 mM), about 175 mM (e.g., 175 mM), about 177.5 mM (e.g., 177.5 mM), about 180 mM (e.g., 180 mM), about 182.5 mM (e.g., 182.5 mM), about 185 mM (e.g., 185 mM), about 187.5 mM (e.g., 187.5 mM), about 190 mM (e.g., 190 mM), about 192.5 mM (e.g., 192.5 mM), about 195 mM (e.g., 195 mM), about 197.5 mM (e.g., 197.5 mM), about 200 mM (e.g., 200 mM), about 202.5 mM (e.g., 202.5 mM), about 205 mM (e.g., 205 mM), about 207.5 mM (e.g., 207.5 mM), about 210 mM (e.g., 210 mM), about 212.5 mM (e.g., 212.5 mM), about 215 mM (e.g., 215 mM), about 217.5 mM (e.g., 217.5 mM), about 220 mM (e.g., 220 mM), about 222.5 mM (e.g., 222.5 mM), about 225 mM (e.g., 225 mM), about 227.5 mM (e.g., 227.5 mM), about 230 mM (e.g., 230 mM), about 232.5 mM (e.g., 232.5 mM), about 235 mM (e.g., 235 mM), about 237.5 mM (e.g., 237.5 mM), about 240 mM (e.g., 240 mM), about 242.5 mM (e.g., 242.5 mM), about 245 mM (e.g., 245 mM), about 247.5 mM (e.g., 247.5 mM), about 250 mM (e.g., 250 mM), or more, of the viscosity reducing agent. In some embodiments, the pharmaceutical composition comprises about 150 mM (e.g., 150 mM) of the viscosity reducing agent.

Non-limiting examples of viscosity reducing agents that may be comprised by pharmaceutical compositions described herein include salts (e.g., metal salts, sodium salts, MgCl₂, NaCl), free amino acids or pharmaceutically acceptable salts thereof (e.g., tryptophan (e.g., L-tryptophan), glycine (e.g., L-glycine), phenylalanine (e.g., L-phenylalanine), methionine (e.g., L-methionine), alanine (e.g., L-alanine), serine (e.g., L-serine), isoleucine (e.g., L-isoleucine), leucine (e.g., L-leucine), threonine (e.g., L-threonine), valine (e.g., L-valine), proline (e.g., L-proline), lysine (e.g., L-lysine), histidine (e.g., L-histidine), glutamine (e.g., L-glutamine), glutamic acid (e.g., L-glutamic acid), arginine (e.g., L-arginine), aspartic acid (e.g., L-aspartic acid), asparagine (e.g., L-asparagine), cysteine (e.g., L-cysteine)), glycinamide or pharmaceutical acceptable salts thereof (e.g., glycinamide hydrochloride), and combinations thereof. A free amino acid may be in the L- or D-stereo isoform, preferably is in the L-stereo isoform. In some embodiments, the viscosity reducing agent is selected from the group consisting of lysine (e.g., L-lysine), arginine (e.g., L-arginine), glycinamide hydrochloride, MgCl₂, NaCl, and a combination thereof.

In some embodiments, the viscosity reducing agent is a free amino acid or a pharmaceutically acceptable thereof (e.g., a hydrochloride salt thereof). In some embodiments, the viscosity reducing agent is arginine (e.g., L-arginine) or lysine (e.g., L-lysine), or a pharmaceutically acceptable salt thereof, preferably lysine (e.g., L-lysine) or a pharmaceutically acceptable salt thereof (e.g., L-lysine HCl).

As demonstrated in the experimental section herein, the present inventors have found that lysine and pharmaceutically acceptable salts thereof is particularly advantageous in that, in addition to a viscosity reducing effect, it enhances the physical stability of pharmaceutical compositions comprising the multispecific antibody or antigen-binding fragments thereof described herein.

In some embodiments, the pharmaceutical composition comprises (in addition to the multispecific, preferably bispecific, antibody or antigen-binding fragment thereof described herein) one or more pharmaceutically acceptable ingredients selected from the group consisting of:
(i) a buffering agent as described earlier herein, preferably a histidine buffer (e.g., L-His/L-His HCl);
(ii) a sugar stabilizer as described earlier herein, preferably trehalose or sucrose (e.g., trehalose, e.g., trehalose dihydrate);
(iii) a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl);
(iv) an anti-oxidant, and;
(v) a surfactant.

Accordingly, in some embodiments, the pharmaceutical composition comprises about 2.5 mM or more (about ≥2.5 mM) arginine (e.g., L-arginine) or lysine (e.g., L-lysine), or a pharmaceutically acceptable salt thereof, preferably lysine (e.g., L-lysine) or a pharmaceutically acceptable salt thereof (e.g., L-lysine HCl), e.g., about 5 mM or more, about 10 mM or more, about 15 mM or more, about 20 mM or more, about 25 mM or more, about 30 mM or more, about 35 mM or more, about 40 mM or more, about 45 mM or more, about 50 mM or more, about 55 mM or more, about 60 mM or more, about 65 mM or more, about 70 mM or more, about 75 mM or more, about 80 mM or more, about 85 mM or more, about 90 mM or more, about 95 mM or more, about 100 mM or more, about 105 mM or more, about 110 mM or more, about 115 mM or more, about 120 mM or more, about 125 mM or more, about 130 mM or more, about 135 mM or more, about 140 mM or more, about 145 mM or more, about 150 mM or more, about 155 mM or more, about 160 mM or more, about 165 mM or more, about 170 mM or more, about 175 mM or more, about 180 mM or more, about 185 mM or more, about 190 mM or more, about 195 mM or more, about 200 mM or more, about 205 mM or more, about 210 mM or more, about 215 mM or more, about 220 mM or more, about 225 mM or more, about 230 mM or more, about 235 mM or more, about 240 mM or more, about 245 mM or more, or about 250 mM or more.

In some embodiments, the pharmaceutical composition comprises from about 2.5 mM to about 500 mM arginine (e.g., L-arginine) or lysine (e.g., L-lysine), or a pharmaceutically acceptable salt thereof, preferably lysine (e.g., L-lysine) or a pharmaceutically acceptable salt thereof (e.g., L-lysine HCl). In some embodiments, the pharmaceutical composition comprises from about 2.5 mM to about 300 mM arginine (e.g., L-arginine) or lysine (e.g., L-lysine), or a pharmaceutically acceptable salt thereof, preferably lysine (e.g., L-lysine) or a pharmaceutically acceptable salt thereof (e.g., L-lysine HCl).

In some embodiments, the pharmaceutical composition comprises from about 100 mM to about 250 mM arginine (e.g., L-arginine) or lysine (e.g., L-lysine), or a pharmaceutically acceptable salt thereof, preferably lysine (e.g., L-lysine) or a pharmaceutically acceptable salt thereof (e.g., L-lysine HCl), e.g., from about 105 mM to about 225 mM, from about 110 mM to about 200 mM, or from about 100 mM, about 115 mM, about 120 mM, about 125 mM to about 175 mM. In some embodiments, the pharmaceutical composition comprises from about 100 mM to about 200 mM arginine (e.g., L-arginine) or lysine (e.g., L-lysine), or a pharmaceutically acceptable salt thereof, preferably lysine (e.g., L-lysine) or a pharmaceutically acceptable salt thereof (e.g., L-lysine HCl). In some embodiments, the pharmaceutical composition comprises from about 100 mM to about 175 mM arginine (e.g., L-arginine) or lysine (e.g., L-lysine), or a pharmaceutically acceptable salt thereof, preferably lysine (e.g., L-lysine) or a pharmaceutically acceptable salt thereof (e.g., L-lysine HCl). In some embodiments, the pharmaceutical composition comprises from about 125 mM to about 175 mM arginine (e.g., L-arginine) or lysine (e.g., L-lysine), or a pharmaceutically acceptable salt thereof, preferably lysine (e.g., L-lysine) or a pharmaceutically acceptable salt thereof (e.g., L-lysine HCl).

In some embodiments, the pharmaceutical composition comprises about 2.5 mM (e.g., 2.5 mM), about 5 mM (e.g., 5 mM), about 7.5 mM (e.g., 7.5 mM), about 10 mM (e.g., 10 mM), about 12.5 mM (e.g., 12.5 mM), about 15 mM (e.g., 15 mM), about 17.5 mM (e.g., 17.5 mM), about 20 mM (e.g., 20 mM), about 22.5 mM (e.g., 22.5 mM), about 25 mM (e.g., 25 mM), about 27.5 mM (e.g., 27.5 mM), about 30 mM (e.g., 30 mM), about 32.5 mM (e.g., 32.5 mM), about 35 mM (e.g., 35 mM), about 37.5 mM (e.g., 37.5 mM), about 40 mM (e.g., 40 mM), about 42.5 mM (e.g., 42.5 mM), about 45 mM (e.g., 45 mM), about 47.5 mM (e.g., 47.5 mM), about 50 mM (e.g., 50 mM), about 52.5 mM (e.g., 52.5 mM), about 55 mM (e.g., 55 mM), about 57.5 mM (e.g., 57.5 mM), about 60 mM (e.g., 60 mM), about 62.5 mM (e.g., 62.5 mM), about 65 mM (e.g., 65 mM), about 67.5 mM (e.g., 67.5 mM), about 70 mM (e.g., 70 mM), about 72.5 mM (e.g., 72.5 mM), about 75 mM (e.g., 75 mM), about 77.5 mM (e.g., 77.5 mM), about 80 mM (e.g., 80 mM), about 82.5 mM (e.g., 82.5 mM), about 85 mM (e.g., 85 mM), about 87.5 mM (e.g., 87.5 mM), about 90 mM (e.g., 90 mM), about 92.5 mM (e.g., 92.5 mM), about 95 mM (e.g., 95 mM), about 97.5 mM (e.g., 97.5 mM), about 100 mM (e.g., 100 mM), about 102.5 mM (e.g., 102.5 mM), about 105 mM (e.g., 105 mM), about 107.5 mM (e.g., 107.5 mM), about 110 mM (e.g., 110 mM), about 112.5 mM (e.g., 112.5 mM), about 115 mM (e.g., 115 mM), about 117.5 mM (e.g., 117.5 mM), about 120 mM (e.g., 120 mM), about 122.5 mM (e.g., 122.5 mM), about 125 mM (e.g., 125 mM), about 127.5 mM (e.g., 127.5 mM), about 130 mM (e.g., 130 mM), about 132.5 mM (e.g., 132.5 mM), about 135 mM (e.g., 135 mM), about 137.5 mM (e.g., 137.5 mM), about 140 mM (e.g., 140 mM), about 142.5 mM (e.g., 142.5 mM), about 145 mM (e.g., 145 mM), about 147.5 mM (e.g., 147.5 mM), about 150 mM (e.g., 150 mM), about 152.5 mM (e.g., 152.5 mM), about 155 mM (e.g., 155 mM), about 157.5 mM (e.g., 157.5 mM), about 160 mM (e.g., 160 mM), about 162.5 mM (e.g., 162.5 mM), about 165 mM (e.g., 165 mM), about 167.5 mM (e.g., 167.5 mM), about 170 mM (e.g., 170 mM), about 172.5 mM (e.g., 172.5 mM), about 175 mM (e.g., 175 mM), about 177.5 mM (e.g., 177.5 mM), about 180 mM (e.g., 180 mM), about 182.5 mM (e.g., 182.5 mM), about 185 mM (e.g., 185 mM), about 187.5 mM (e.g., 187.5 mM), about 190 mM (e.g., 190 mM), about 192.5 mM (e.g., 192.5 mM), about 195 mM (e.g., 195 mM), about 197.5 mM (e.g., 197.5 mM), about 200 mM (e.g., 200 mM), about 202.5 mM (e.g., 202.5 mM), about 205 mM (e.g., 205 mM), about 207.5 mM (e.g., 207.5 mM), about 210 mM (e.g., 210 mM), about 212.5 mM (e.g., 212.5 mM), about 215 mM (e.g., 215 mM), about 217.5 mM (e.g., 217.5 mM), about 220 mM (e.g., 220 mM), about 222.5 mM (e.g., 222.5 mM), about 225 mM (e.g., 225 mM), about 227.5 mM (e.g., 227.5 mM), about 230 mM (e.g., 230 mM), about 232.5 mM (e.g., 232.5 mM), about 235 mM (e.g., 235 mM), about 237.5 mM (e.g., 237.5 mM), about 240 mM (e.g., 240 mM), about 242.5 mM (e.g., 242.5 mM), about 245 mM (e.g., 245 mM), about 247.5 mM (e.g., 247.5 mM), about 250 mM (e.g., 250 mM), or more, arginine (e.g., L-arginine) or lysine (e.g., L-lysine), or a pharmaceutically acceptable salt thereof, preferably lysine (e.g., L-lysine) or a pharmaceutically acceptable salt thereof (e.g., L-lysine HCl). In some embodiments, the pharmaceutical composition comprises about 150 mM (e.g., 150 mM) arginine (e.g., L-arginine) or lysine (e.g., L-lysine), or a pharmaceutically acceptable salt thereof, preferably lysine (e.g., L-lysine) or a pharmaceutically acceptable salt thereof (e.g., L-lysine HCl).

### Anti-Oxidant

In preferred embodiments, the pharmaceutical, preferably liquid (e.g., aqueous), composition described herein comprises an anti-oxidant. As demonstrated in the experimental section herein, the present inventors have found that inclusion of an anti-oxidant in pharmaceutical compositions decreases the oxidation of the multispecific antibody or antigen-binding fragment thereof relative to pharmaceutical compositions not comprising an anti-oxidant. Multispecific antibody oxidation can impact its structure, functional and biological activity, for example by increasing aggregation. Without wishing to be bound by theory, oxidation of an amino acid residue comprised by a CDR, e.g., HCDR1, HCDR2, HCDR3, LCDR1, LCDR2, or LCDR3, could be particularly detrimental as it may affect the biological activity of the multispecific antibody or antigen-binding fragment thereof.

In embodiments wherein the first part that binds to IL-18 comprises a first variable light region (VL1) comprising SEQ ID NO: 13 and the second part that binds to IL-13 comprises a second variable light region comprising SEQ ID NO: 27, the M37 residue in SEQ ID NO: 27 (corresponding to LCDR1 of VL2; numbering starting from D1 of SEQ ID NO: 27) may be particularly susceptible to oxidation. Similarly, in embodiments wherein the first part that binds to IL-18 comprises a first light chain (LC1) comprising SEQ ID NO: 14 and the second part that binds to IL-13 comprises a second light chain (LC2) comprising SEQ ID NO: 28, the M37 residue in SEQ ID NO: 28 (corresponding to LCDR1 of LC2; numbering starting from D1 of SEQ ID NO: 28) may be particularly susceptible to oxidation.

In some embodiments, the anti-oxidant is a metal chelator, e.g., EDTA (ethylenediamine tetraacetic acid) or DTPA (diethylenetriaminepentaacetic acid). In preferred embodiments, the anti-oxidant is methionine (e.g., L-methionine) or a pharmaceutically acceptable salt thereof, e.g., a hydrochloride salt.

In some embodiments, the pharmaceutical composition comprises (in addition to the multispecific, preferably bispecific, antibody or antigen-binding fragment thereof described herein) one or more pharmaceutically acceptable ingredients selected from the group consisting of:
(i) a buffering agent as described earlier herein, preferably a histidine buffer (e.g., L-His/L-His HCl);
(ii) a sugar stabilizer as described earlier herein, preferably trehalose or sucrose (e.g., trehalose, e.g., trehalose dihydrate);
(iii) a viscosity reducing agent as described earlier herein, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl);
(iv) an anti-oxidant, preferably methionine (e.g., L-methionine) or a pharmaceutically acceptable salt thereof, and;
(v) a surfactant.

In some embodiments, the pharmaceutical composition comprises about 1 mM or more (about ≥1 mM) methionine (e.g., L-methionine) or a pharmaceutically acceptable salt thereof, e.g., about 1.5 mM or more, about 2 mM or more, about 2.5 mM or more, about 3 mM or more, about 3.5 mM or more, about 4 mM or more, about 4.5 mM or more, about 5 mM or more, about 5.5 mM or more, about 6 mM or more, about 6.5 mM or more, about 7 mM or more, about 7.5 mM or more, about 8 mM or more, about 8.5 mM or more, about 9 mM or more, about 9.5 mM or more, about 10 mM or more, about 10.5 mM or more, about 11 mM or more, about 11.5 mM or more, about 12 mM or more, about 12.5 mM or more, about 13 mM or more, about 13.5 mM or more, about 14 mM or more, about 14.5 mM or more, about 15 mM or more, about 15.5 mM or more, about 16 mM or more, about 16.5 mM or more, about 17 mM or more, about 17.5 mM or more, about 18 mM or more, about 18.5 mM or more, about 19 mM or more, about 19.5 mM or more, or about 20 mM or more. In some embodiments, the pharmaceutical composition comprises about 5 mM or more (e.g., 5 mM) methionine (e.g., L-methionine) or a pharmaceutically acceptable salt thereof.

In some embodiments, the pharmaceutical composition comprises from about 1 mM to about 250 mM methionine (e.g., L-methionine) or a pharmaceutically acceptable salt thereof. In some embodiments, the pharmaceutical composition comprises from about 1 mM to about 225 mM methionine (e.g., L-methionine) or a pharmaceutically acceptable salt thereof. In some embodiments, the pharmaceutical composition comprises from about 1 mM to about 200 mM methionine (e.g., L-methionine) or a pharmaceutically acceptable salt thereof. In some embodiments, the pharmaceutical composition comprises from about 1 mM to about 175 mM methionine (e.g., L-methionine) or a pharmaceutically acceptable salt thereof. In some embodiments, the pharmaceutical composition comprises from about 1 mM to about 150 mM methionine (e.g., L-methionine) or a pharmaceutically acceptable salt thereof. In some embodiments, the pharmaceutical composition comprises from about 1 mM to about 125 mM methionine (e.g., L-methionine) or a pharmaceutically acceptable salt thereof. In some embodiments, the pharmaceutical composition comprises from about 1 mM to about 100 mM methionine (e.g., L-methionine) or a pharmaceutically acceptable salt thereof. In some embodiments, the pharmaceutical composition comprises from about 1 mM to about 75 mM methionine (e.g., L-methionine) or a pharmaceutically acceptable salt thereof. In some embodiments, the pharmaceutical composition comprises from about 1 mM to about 50 mM methionine (e.g., L-methionine) or a pharmaceutically acceptable salt thereof.

In some embodiments, the pharmaceutical composition comprises from about 2.5 mM to about 20 mM methionine (e.g., L-methionine) or a pharmaceutically acceptable salt thereof, e.g., from about 2.5 mM to about 15 mM, from about 2.5 mM to about 10 mM, from about 3 mM to about 9 mM, from about 3.5 mM to about 8 mM, from about 4 mM to about 7 mM, or from about 4.5 mM to about 6 mM. In some embodiments, the pharmaceutical composition comprises from about 2.5 mM to about 7.5 mM methionine (e.g., L-methionine) or a pharmaceutically acceptable salt thereof, e.g., about 5 mM (e.g., 5 mM).

### Surfactant

In some embodiments, the pharmaceutical, preferably liquid (e.g., aqueous), composition comprises a surfactant. The term “surfactant” generally refers to organic substances having amphipathic structures; i.e., they are typically composed of groups of opposing solubility tendencies, typically an oil-soluble hydrocarbon chain and a water-soluble ionic group. Surfactants can be classified, depending on the charge of the surface-active moiety, into non-ionic, anionic, cationic and dispersing agents for various pharmaceutical compositions and preparations of biological materials. Suitable surfactants include, but are not limited to, non-ionic surfactants, ionic surfactants and zwitterionic surfactants.

Non-limiting examples of surfactants include, but are not limited to, sorbitan fatty acid esters (e.g., sorbitan monocaprylate, sorbitan monolaurate, sorbitan monopalmitate), sorbitan trioleate, glycerine fatty acid esters (e.g., glycerine monocaprylate, glycerine monomyristate, glycerine monostearate), polyglycerine fatty acid esters (e.g., decaglyceryl monostearate, decaglyceryl distearate, decaglyceryl monolinoleate), polyoxyethylene sorbitan fatty acid esters (sorbitan monolaurate, polyoxyethylene sorbitan monooleate, e.g., polyoxyethylene polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g., polyoxyethylene glyceryl monostearate), polyethylene glycol fatty acid esters (e.g., polyethylene glycol distearate), polyoxyethylene alkyl ethers (e.g., polyoxyethylene lauryl ether), polyoxyethylene polyoxypropylene alkyl ethers (e.g., polyoxyethylene polyoxypropylene glycol, polyoxyethylene polyoxypropylene propyl ether, polyoxyethylene polyoxypropylene cetyl ether), polyoxyethylene alkylphenyl ethers (e.g., polyoxyethylene nonylphenyl ether), polyoxyethylene hydrogenated castor oils (e.g., polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), polyoxyethylene fatty acid amides (e.g., polyoxyethylene stearic acid amide), C₁₀-C₁₈ alkyl sulfates (e.g., sodium cetyl sulfate, sodium lauryl sulfate, sodium oleyl sulfate), polyoxyethylene C₁₀-C₁₈ alkyl ether sulfate with an average of 2 to 4 moles of ethylene oxide units added (e.g., sodium polyoxyethylene lauryl sulfate), C₁-C₁₈ alkyl sulfosuccinate ester salts (e.g., sodium lauryl sulfosuccinate ester), natural surfactants such as lecithin, glycerophospholipid, sphingophospholipids (e.g., sphingomyelin), sucrose esters of C₁₂-C₁₈ fatty acids, polysorbate 20, polysorbate 40, polysorbate 60, polysorbate 80, and poloxamer (Polox) 188. A composition may e.g., include one or more of the abovementioned surfactants. Among surfactants, preferred are polyoxyethylene sorbitan fatty acid esters e.g., polysorbate 20, 40, 60 or 80, and Polox (Poloxamer) 188, with polysorbate 80 (PS80) being more preferred.

Accordingly, in some embodiments, the pharmaceutical composition comprises a surfactant selected from the group consisting of polysorbate 80 (PS80), polysorbate 20 (PS20), Polox188, and a combination thereof, preferably polysorbate 80 (PS80). In some embodiments, the polysorbate 80 (PS80), polysorbate 20 (PS20), or Polox188, preferably polysorbate 80 (PS80), has low peroxide content, e.g., has a peroxide content of about ≤2 meqO2/kg, about ≤1.9 meqO2/kg, about ≤1.8 meqO2/kg, about ≤1.7 meqO2/kg, about ≤1.6 meqO2/kg, about ≤1.5 meqO2/kg, about ≤1.4 meqO2/kg, about ≤1.3 meqO2/kg, about ≤1.2 meqO2/kg, about ≤1.1 meqO2/kg, about ≤1 meqO2/kg, about ≤0.9 meqO2/kg, about ≤0.8 meqO2/kg, about ≤0.7 meqO2/kg, about ≤0.6 meqO2/kg, about ≤0.5 meqO2/kg, about ≤0.4 meqO2/kg, about ≤0.3 meqO2/kg, about ≤0.2 meqO2/kg, or about ≤0.1 meqO2/kg.

In some embodiments, the pharmaceutical composition comprises (in addition to the multispecific antibody or antigen-binding fragment thereof described herein) one or more pharmaceutically acceptable ingredients selected from the group consisting of:
(i) a buffering agent as described earlier herein, preferably a histidine buffer (e.g., L-His/L-His HCl);
(ii) a sugar stabilizer as described earlier herein, preferably trehalose or sucrose (e.g., trehalose, e.g., trehalose dihydrate);
(iii) a viscosity reducing agent as described earlier herein, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl);
(iv) an anti-oxidant as described earlier herein, preferably methionine (e.g., L-methionine) or a pharmaceutically acceptable salt thereof, and;
(v) a surfactant, e.g., selected from the group consisting of polysorbate 80 (PS80), polysorbate 20 (PS20), Polox188, preferably polysorbate 80 (PS80).

In some embodiments, the pharmaceutical composition comprises about 0.01% (w/v) or more (≥0.01% w/v) of the surfactant (e.g., polysorbate 80 (PS80), polysorbate 20 (PS20), Polox188, preferably polysorbate 80 (PS80)), e.g., about 0.02% or more (e.g., 0.02%), about 0.03% or more (e.g., 0.03%), about 0.04% or more (e.g., 0.04%), about 0.05% or more (e.g., 0.05%), about 0.06% or more (e.g., 0.06%), about 0.07% or more (e.g., 0.07%), about 0.08% or more (e.g., 0.08%), about 0.09% or more (e.g., 0.09%), about 0.1% or more (e.g., 0.1%), about 0.11% or more (e.g., 0.11%), about 0.12% or more (e.g., 0.12%), about 0.13% or more (e.g., 0.13%), about 0.14% or more (e.g., 0.14%), about 0.15% or more (e.g., 0.15%), about 0.16% or more (e.g., 0.16%), about 0.17% or more (e.g., 0.17%), about 0.18% or more (e.g., 0.18%), about 0.19% or more (e.g., 0.19%), about 0.2% or more (e.g., 0.2%), about 0.21% or more (e.g., 0.21%), about 0.22% or more (e.g., 0.22%), about 0.23% or more (e.g., 0.23%), about 0.24% or more (e.g., 0.24%), about 0.25% or more (e.g., 0.25%), about 0.26% or more (e.g., 0.26%), about 0.27% or more (e.g., 0.27%), about 0.28% or more (e.g., 0.28%), about 0.29% or more (e.g., 0.29%), about 0.3% or more (e.g., 0.3%), about 0.31% or more (e.g., 0.31%), about 0.32% or more (e.g., 0.32%), about 0.33% or more (e.g., 0.33%), about 0.34% or more (e.g., 0.34%), about 0.35% or more (e.g., 0.35%), about 0.36% or more (e.g., 0.36%), about 0.37% or more (e.g., 0.37%), about 0.38% or more (e.g., 0.38%), about 0.39% or more (e.g., 0.39%), about 0.4% or more (e.g., 0.4%), about 0.41% or more (e.g., 0.41%), about 0.42% or more (e.g., 0.42%), about 0.43% or more (e.g., 0.43%), about 0.44% or more (e.g., 0.44%), about 0.45% or more (e.g., 0.45%), about 0.46% or more (e.g., 0.46%), about 0.47% or more (e.g., 0.47%), about 0.48% or more (e.g., 0.48%), about 0.49% or more (e.g., 0.49%), or about 0.5% or more (e.g., 0.5%). In some embodiments, the pharmaceutical composition comprises about 0.04% (w/v) (e.g., 0.04%) of the surfactant (e.g., polysorbate 80 (PS80), polysorbate 20 (PS20), Polox188, preferably polysorbate 80 (PS80)),

In some embodiments, the pharmaceutical composition comprises from about 0.01% (w/v) to about 0.5% (w/v) of the surfactant (e.g., polysorbate 80 (PS80), polysorbate 20 (PS20), Polox188, preferably polysorbate 80 (PS80)).

In some embodiments, the pharmaceutical composition comprises from about 0.01% (w/v) to about 0.1% (w/v) of the surfactant (e.g., polysorbate 80 (PS80), polysorbate 20 (PS20), Polox188, preferably polysorbate 80 (PS80)), e.g., from about 0.02% (w/v) to 0.08% (w/v).

In some embodiments, the pharmaceutical composition comprises from about 0.02% (w/v) to about 0.08% (w/v) of the surfactant (e.g., polysorbate 80 (PS80), polysorbate 20 (PS20), Polox188, preferably polysorbate 80 (PS80)), e.g., from about 0.02% (w/v) to about 0.07% (w/v), from about 0.03% (w/v) to about 0.06% (w/v), or from about 0.03% (w/v) to about 0.05% (w/v).

### Other Ingredients

The pharmaceutical, preferably liquid (e.g., aqueous), composition described herein may comprise one or more additional pharmaceutically acceptable ingredients, including, for example, flavoring agents, antimicrobial agents, sweeteners, antistatic agents, lipids such as phospholipids or fatty acids, steroids such as cholesterol, protein excipients such as serum albumin (human serum albumin), recombinant human albumin, gelatin, casein, salt-forming counterions such sodium and the like. Examples of other ingredients suitable for use in the pharmaceutical compositions may be found in e.g., Remington: The Science and Practice of Pharmacy, 23rd edition (2020), Elsevier, Netherlands.

The pharmaceutical composition may further comprise an additional active ingredient in addition to the multispecific antibody or antigen-binding fragment thereof, e.g., in the case of combination therapies described later herein.

### Exemplary Pharmaceutical Compositions

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20;
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28;
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28;
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20;
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28;
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28;
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20;
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28;
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) SEQ ID NO: 58 and a second light chain (LC2) comprising SEQ ID NO: 28;
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20;
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28;
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28;
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20;
from about 5 mM to about 50 mM, e.g., from about 10 mM to about 25 mM (e.g., about 20 mM), of a histidine buffer (e.g., L-histidine buffer, e.g., L-His/L-His HCl buffer).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
from about 5 mM to about 50 mM, e.g., from about 10 mM to about 25 mM (e.g., about 20 mM), of a histidine buffer (e.g., L-histidine buffer, e.g., L-His/L-His HCl buffer).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14;
ii) a second part that binds to IL-13 comprising a second heavy chain (HC2) comprising SEQ ID NO: 56 and a second light chain (LC2) comprising SEQ ID NO: 28;
from about 5 mM to about 50 mM, e.g., from about 10 mM to about 25 mM (e.g., about 20 mM), of a histidine buffer (e.g., L-histidine buffer, e.g., L-His/L-His HCl buffer).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28;
from about 5 mM to about 50 mM, e.g., from about 10 mM to about 25 mM (e.g., about 20 mM), of a histidine buffer (e.g., L-histidine buffer, e.g., L-His/L-His HCl buffer).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20;
from about 5 mM to about 50 mM, e.g., from about 10 mM to about 25 mM (e.g., about 20 mM), of a histidine buffer (e.g., L-histidine buffer, e.g., L-His/L-His HCl buffer);
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
from about 5 mM to about 50 mM, e.g., from about 10 mM to about 25 mM (e.g., about 20 mM), of a histidine buffer (e.g., L-histidine buffer, e.g., L-His/L-His HCl buffer);
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14;
ii) a second part that binds to IL-13 comprising a second heavy chain (HC2) comprising SEQ ID NO: 56 and a second light chain (LC2) comprising SEQ ID NO: 28;
from about 5 mM to about 50 mM, e.g., from about 10 mM to about 25 mM (e.g., about 20 mM), of a histidine buffer (e.g., L-histidine buffer, e.g., L-His/L-His HCl buffer);
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28;
from about 5 mM to about 50 mM, e.g., from about 10 mM to about 25 mM (e.g., about 20 mM), of a histidine buffer (e.g., L-histidine buffer, e.g., L-His/L-His HCl buffer);
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl), and;
from about 2.5 mM to about 20 mM, e.g., from about 2.5 mM to about 7.5 mM (e.g., about 5 mM), methionine (e.g., L-methionine) or a pharmaceutically acceptable salt thereof.

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20;
from about 0.01% (w/v) to about 0.1% (w/v), e.g., from about 0.02% (w/v) to about 0.08% (w/v) (e.g., about 0.04% (w/v)), of a surfactant, e.g., selected from the group consisting of polysorbate 80 (PS80), polysorbate 20 (PS20), Polox188, preferably polysorbate 80 (PS80).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
from about 0.01% (w/v) to about 0.1% (w/v), e.g., from about 0.02% (w/v) to about 0.08% (w/v) (e.g., about 0.04% (w/v)), of a surfactant, e.g., selected from the group consisting of polysorbate 80 (PS80), polysorbate 20 (PS20), Polox188, preferably polysorbate 80 (PS80).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14;
ii) a second part that binds to IL-13 comprising a second heavy chain (HC2) comprising SEQ ID NO: 56 and a second light chain (LC2) comprising SEQ ID NO: 28;
from about 0.01% (w/v) to about 0.1% (w/v), e.g., from about 0.02% (w/v) to about 0.08% (w/v) (e.g., about 0.04% (w/v)), of a surfactant, e.g., selected from the group consisting of polysorbate 80 (PS80), polysorbate 20 (PS20), Polox188, preferably polysorbate 80 (PS80).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28;
from about 0.01% (w/v) to about 0.1% (w/v), e.g., from about 0.02% (w/v) to about 0.08% (w/v) (e.g., about 0.04% (w/v)), of a surfactant, e.g., selected from the group consisting of polysorbate 80 (PS80), polysorbate 20 (PS20), Polox188, preferably polysorbate 80 (PS80).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises or consists essentially of:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20;
from about 5 mM to about 50 mM, e.g., from about 10 mM to about 25 mM (e.g., about 20 mM), of a histidine buffer (e.g., L-histidine buffer, e.g., L-His/L-His HCl buffer);
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl);
from about 2.5 mM to about 20 mM, e.g., from about 2.5 mM to about 7.5 mM (e.g., about 5 mM), methionine (e.g., L-methionine) or a pharmaceutically acceptable salt thereof, and;
from about 0.01% (w/v) to about 0.1% (w/v), e.g., from about 0.02% (w/v) to about 0.08% (w/v) (e.g., about 0.04% (w/v)), of a surfactant, e.g., selected from the group consisting of polysorbate 80 (PS80), polysorbate 20 (PS20), Polox188, preferably polysorbate 80 (PS80).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises or consists essentially of:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
from about 5 mM to about 50 mM, e.g., from about 10 mM to about 25 mM (e.g., about 20 mM), of a histidine buffer (e.g., L-histidine buffer, e.g., L-His/L-His HCl buffer);
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl);
from about 2.5 mM to about 20 mM, e.g., from about 2.5 mM to about 7.5 mM (e.g., about 5 mM), methionine (e.g., L-methionine) or a pharmaceutically acceptable salt thereof, and;
from about 0.01% (w/v) to about 0.1% (w/v), e.g., from about 0.02% (w/v) to about 0.08% (w/v) (e.g., about 0.04% (w/v)), of a surfactant, e.g., selected from the group consisting of polysorbate 80 (PS80), polysorbate 20 (PS20), Polox188, preferably polysorbate 80 (PS80).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises or consists essentially of:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14;
ii) a second part that binds to IL-13 comprising a second heavy chain (HC2) comprising SEQ ID NO: 56 and a second light chain (LC2) comprising SEQ ID NO: 28;
from about 5 mM to about 50 mM, e.g., from about 10 mM to about 25 mM (e.g., about 20 mM), of a histidine buffer (e.g., L-histidine buffer, e.g., L-His/L-His HCl buffer);
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl);
from about 2.5 mM to about 20 mM, e.g., from about 2.5 mM to about 7.5 mM (e.g., about 5 mM), methionine (e.g., L-methionine) or a pharmaceutically acceptable salt thereof, and;
from about 0.01% (w/v) to about 0.1% (w/v), e.g., from about 0.02% (w/v) to about 0.08% (w/v) (e.g., about 0.04% (w/v)), of a surfactant, e.g., selected from the group consisting of polysorbate 80 (PS80), polysorbate 20 (PS20), Polox188, preferably polysorbate 80 (PS80).

In some embodiments, the pharmaceutical, preferably liquid (e.g., aqueous) composition has a pH from about 5 to about 8, e.g., from about 5.5 to about 7.4 or from about 6 to about 7 (e.g., about 6.5), and comprises or consists essentially of:
from about 15 mg/mL to about 200 mg/mL, e.g., from about 100 mg/mL to about 175 mg/mL (e.g., about 150 mg/mL), of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28;
from about 5 mM to about 50 mM, e.g., from about 10 mM to about 25 mM (e.g., about 20 mM), of a histidine buffer (e.g., L-histidine buffer, e.g., L-His/L-His HCl buffer);
from about 50 mM to about 220 mM, e.g., about 100 mM to about 150 mM (e.g., about 120 mM), of a sugar stabilizer, preferably trehalose or sucrose (e.g., trehalose, e.g., trehalose dihydrate);
from about 100 mM to about 175 mM (e.g. about 150 mM) of a viscosity reducing agent, preferably arginine (e.g., L-arginine) or lysine (e.g., L-lysine), or a pharmaceutically acceptable salt thereof, more preferably lysine (e.g., L-lysine) or a pharmaceutically acceptable salt thereof (e.g., L-lysine HCl);
from about 2.5 mM to about 20 mM, e.g., from about 2.5 mM to about 7.5 mM (e.g., about 5 mM), methionine (e.g., L-methionine) or a pharmaceutically acceptable salt thereof, and;
from about 0.01% (w/v) to about 0.1% (w/v), e.g., from about 0.02% (w/v) to about 0.08% (w/v) (e.g., about 0.04% (w/v)), of a surfactant, e.g., selected from the group consisting of polysorbate 80 (PS80), polysorbate 20 (PS20), Polox188, preferably polysorbate 80 (PS80).

In some embodiments, the pharmaceutical, preferably liquid (e.g., aqueous) composition has a pH from about 6 to about 7 (e.g., about 6.5), and comprises or consists essentially of:
from about 100 mg/mL to about 175 mg/mL of a bispecific antibody or antigen-binding fragment thereof, wherein the bispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20;
from about 10 mM to about 25 mM of a histidine buffer (e.g., L-His/L-His HCl buffer);
from about 100 mM to about 150 mM trehalose (e.g., trehalose dihydrate);
from about 100 mM to about 175 mM lysine (e.g., L-lysine) or a pharmaceutically acceptable salt thereof (e.g., L-lysine HCl);
from about 2.5 mM to about 20 mM methionine (e.g., L-methionine) or a pharmaceutically acceptable salt thereof, and;
from about 0.02% (w/v) to about 0.08% (w/v) polysorbate 80 (PS80).

In some embodiments, the pharmaceutical, preferably liquid (e.g., aqueous) composition has a pH of about 6.5, and comprises or consists essentially of:
about 150 mg/mL of a bispecific antibody or antigen-binding fragment thereof, wherein the bispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20;
about 20 mM of a histidine buffer (e.g., L-His/L-His HCl buffer);
about 120 mM trehalose (e.g., trehalose dihydrate);
about 150 mM lysine (e.g., L-lysine) or a pharmaceutically acceptable salt thereof (e.g., L-lysine HCl);
about 5 mM methionine (e.g., L-methionine) or a pharmaceutically acceptable salt thereof, and;
about 0.04% (w/v) polysorbate 80 (PS80).

In some embodiments, the pharmaceutical, preferably liquid (e.g., aqueous) composition has a pH from about 6 to about 7 (e.g., about 6.5), and comprises or consists essentially of:
from about 100 mg/mL to about 175 mg/mL of a bispecific antibody or antigen-binding fragment thereof, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
from about 10 mM to about 25 mM of a histidine buffer (e.g., L-His/L-His HCl buffer);
from about 100 mM to about 150 mM trehalose (e.g., trehalose dihydrate);
from about 100 mM to about 175 mM lysine (e.g., L-lysine) or a pharmaceutically acceptable salt thereof (e.g., L-lysine HCl);
from about 2.5 mM to about 20 mM methionine (e.g., L-methionine) or a pharmaceutically acceptable salt thereof, and;
from about 0.02% (w/v) to about 0.08% (w/v) polysorbate 80 (PS80).

In some embodiments, the pharmaceutical, preferably liquid (e.g., aqueous) composition has a pH of from about 6 to about 7 (e.g., about 6.5), and comprises or consists essentially of:
about 75 mg/mL or more of a bispecific antibody or antigen-binding fragment thereof, e.g., about 100 mg/mL or more, about 125 mg/mL or more, or about 150 mg/mL or more, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
about 5 mM or more of a histidine buffer (e.g., L-His/L-His HCl buffer), e.g., about 10 mM or more, about 15 mM or more, or about 20 mM or more;
about 50 mM or more trehalose (e.g., trehalose dihydrate), e.g., about 60 mM or more, about 70 mM or more, about 80 mM or more, about 90 mM or more, about 100 mM or more, about 110 mM or more, or about 120 mM or more;
about 100 mM or more lysine (e.g., L-lysine) or a pharmaceutically acceptable salt thereof (e.g., L-lysine HCl), e.g., about 110 mM or more, about 120 mM or more, about 130 mM or more, about 140 mM or more, or about 150 mM or more;
about 2.5 mM or more methionine (e.g., L-methionine) or a pharmaceutically acceptable salt thereof, e.g., about 3 mM or more, about 3.5 mM or more, about 4 mM or more, about 4.5 mM or more, or about 5 mM or more, and;
about 0.01% (w/v) or more polysorbate 80 (PS80), e.g., about 0.02% (w/v) or more, about 0.03% (w/v) or more, or about 0.04% (w/v) or more.

In some embodiments, the pharmaceutical, preferably liquid (e.g., aqueous) composition has a pH of about 6.5, and comprises or consists essentially of:
about 150 mg/mL of a bispecific antibody or antigen-binding fragment thereof, wherein the bispecific antibody comprises:
i) a first part that binds to IL-18 comprising a first variable heavy region (VH1) comprising SEQ ID NO: 41 and a first variable light region (VL1) comprising SEQ ID NO: 13, and;
ii) a second part that binds to IL-13 comprising a second variable heavy region (VH2) comprising SEQ ID NO: 55 and a second variable light region (VL2) comprising SEQ ID NO: 27;
about 20 mM of a histidine buffer (e.g., L-His/L-His HCl buffer);
about 120 mM trehalose (e.g., trehalose dihydrate);
about 150 mM lysine (e.g., L-lysine) or a pharmaceutically acceptable salt thereof (e.g., L-lysine HCl);
about 5 mM methionine (e.g., L-methionine) or a pharmaceutically acceptable salt thereof, and;
about 0.04% (w/v) polysorbate 80 (PS80).

In some embodiments, the pharmaceutical, preferably liquid (e.g., aqueous) composition has a pH from about 6 to about 7 (e.g., about 6.5), and comprises or consists essentially of:
from about 100 mg/mL to about 175 mg/mL of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14;
ii) a second part that binds to IL-13 comprising a second heavy chain (HC2) comprising SEQ ID NO: 56 and a second light chain (LC2) comprising SEQ ID NO: 28;
from about 10 mM to about 25 mM of a histidine buffer (e.g., L-His/L-His HCl buffer);
from about 100 mM to about 150 mM trehalose (e.g., trehalose dihydrate);
from about 100 mM to about 175 mM lysine (e.g., L-lysine) or a pharmaceutically acceptable salt thereof (e.g., L-lysine HCl);
from about 2.5 mM to about 20 mM methionine (e.g., L-methionine) or a pharmaceutically acceptable salt thereof, and;
from about 0.02% (w/v) to about 0.08% (w/v) polysorbate 80 (PS80).

In some embodiments, the pharmaceutical, preferably liquid (e.g., aqueous) composition has a pH of from about 6 to about 7 (e.g., about 6.5), and comprises or consists essentially of:
about 75 mg/mL or more of a bispecific antibody or antigen-binding fragment thereof, e.g., about 100 mg/mL or more, about 125 mg/mL or more, or about 150 mg/mL or more, wherein the bispecific antibody comprises:
i) a first part that binds to IL-18 comprising a first heavy chain (HCl) comprising SEQ ID NO: 42 and a first light chain (LC1) comprising SEQ ID NO: 14;
ii) a second part that binds to IL-13 comprising a second heavy chain (HC2) comprising SEQ ID NO: 56 and a second light chain (LC2) comprising SEQ ID NO: 28;
about 5 mM or more of a histidine buffer (e.g., L-His/L-His HCl buffer), e.g., about 10 mM or more, about 15 mM or more, or about 20 mM or more;
about 50 mM or more trehalose (e.g., trehalose dihydrate), e.g., about 60 mM or more, about 70 mM or more, about 80 mM or more, about 90 mM or more, about 100 mM or more, about 110 mM or more, or about 120 mM or more;
about 100 mM or more lysine (e.g., L-lysine) or a pharmaceutically acceptable salt thereof (e.g., L-lysine HCl), e.g., about 110 mM or more, about 120 mM or more, about 130 mM or more, about 140 mM or more, or about 150 mM or more;
about 2.5 mM or more methionine (e.g., L-methionine) or a pharmaceutically acceptable salt thereof, e.g., about 3 mM or more, about 3.5 mM or more, about 4 mM or more, about 4.5 mM or more, or about 5 mM or more, and;
about 0.01% (w/v) or more polysorbate 80 (PS80), e.g., about 0.02% (w/v) or more, about 0.03% (w/v) or more, or about 0.04% (w/v) or more.

In some embodiments, the pharmaceutical, preferably liquid (e.g., aqueous) composition has a pH of about 6.5, and comprises or consists essentially of:
about 150 mg/mL of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14;
ii) a second part that binds to IL-13 comprising a second heavy chain (HC2) comprising SEQ ID NO: 56 and a second light chain (LC2) comprising SEQ ID NO: 28;
about 20 mM of a histidine buffer (e.g., L-His/L-His HCl buffer);
about 120 mM trehalose (e.g., trehalose dihydrate);
about 150 mM lysine (e.g., L-lysine) or a pharmaceutically acceptable salt thereof (e.g., L-lysine HCl);
about 5 mM methionine (e.g., L-methionine) or a pharmaceutically acceptable salt thereof, and;
about 0.04% (w/v) polysorbate 80 (PS80).

In some embodiments, the pharmaceutical, preferably liquid (e.g., aqueous) composition has a pH of 6.5, and comprises or consists essentially of:
150 mg/mL of a bispecific antibody or antigen-binding fragment thereof, wherein the bispecific antibody comprises:
i) a first part that binds to IL-18 comprising a first heavy chain (HCl) comprising SEQ ID NO: 42 and a first light chain (LC1) comprising SEQ ID NO: 14;
ii) a second part that binds to IL-13 comprising a second heavy chain (HC2) comprising SEQ ID NO: 56 and a second light chain (LC2) comprising SEQ ID NO: 28;
20 mM of a histidine buffer (e.g., L-His/L-His HCl buffer);
120 mM trehalose (e.g., trehalose dihydrate);
150 mM lysine (e.g., L-lysine) or a pharmaceutically acceptable salt thereof (e.g., L-lysine HCl);
5 mM methionine (e.g., L-methionine) or a pharmaceutically acceptable salt thereof, and;
0.04% (w/v) polysorbate 80 (PS80).

In some embodiments, the pharmaceutical, preferably liquid (e.g., aqueous) composition has a pH of from about 6 to about 7 (e.g., about 6.5), and comprises or consists essentially of:
from about 100 mg/mL to about 175 mg/mL of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14;
ii) a second part that binds to IL-13 comprising a second heavy chain (HC2) comprising SEQ ID NO: 58 and a second light chain (LC2) comprising SEQ ID NO: 28;
from about 10 mM to about 25 mM of a histidine buffer (e.g., L-His/L-His HCl buffer);
from about 100 mM to about 150 mM trehalose (e.g., trehalose dihydrate);
from about 100 mM to about 175 mM lysine (e.g., L-lysine) or a pharmaceutically acceptable salt thereof (e.g., L-lysine HCl);
from about 2.5 mM to about 20 mM methionine (e.g., L-methionine) or a pharmaceutically acceptable salt thereof, and;
from about 0.02% (w/v) to about 0.08% (w/v) polysorbate 80 (PS80).

In some embodiments, the pharmaceutical, preferably liquid (e.g., aqueous) composition has a pH of from about 6 to about 7 (e.g., about 6.5), and comprises or consists essentially of:
about 75 mg/mL or more of a bispecific antibody or antigen-binding fragment thereof, e.g., about 100 mg/mL or more, about 125 mg/mL or more, or about 150 mg/mL or more, wherein the bispecific antibody comprises:
i) a first part that binds to IL-18 comprising a first heavy chain (HCl) comprising SEQ ID NO: 57 and a first light chain (LC1) comprising SEQ ID NO: 14;
ii) a second part that binds to IL-13 comprising a second heavy chain (HC2) comprising SEQ ID NO: 58 and a second light chain (LC2) comprising SEQ ID NO: 28;
about 5 mM or more of a histidine buffer (e.g., L-His/L-His HCl buffer), e.g., about 10 mM or more, about 15 mM or more, or about 20 mM or more;
about 50 mM or more trehalose (e.g., trehalose dihydrate), e.g., about 60 mM or more, about 70 mM or more, about 80 mM or more, about 90 mM or more, about 100 mM or more, about 110 mM or more, or about 120 mM or more;
about 100 mM or more lysine (e.g., L-lysine) or a pharmaceutically acceptable salt thereof (e.g., L-lysine HCl), e.g., about 110 mM or more, about 120 mM or more, about 130 mM or more, about 140 mM or more, or about 150 mM or more;
about 2.5 mM or more methionine (e.g., L-methionine) or a pharmaceutically acceptable salt thereof, e.g., about 3 mM or more, about 3.5 mM or more, about 4 mM or more, about 4.5 mM or more, or about 5 mM or more, and;
about 0.01% (w/v) or more polysorbate 80 (PS80), e.g., about 0.02% (w/v) or more, about 0.03% (w/v) or more, or about 0.04% (w/v) or more.

In some embodiments, the pharmaceutical, preferably liquid (e.g., aqueous) composition has a pH of about 6.5, and comprises or consists essentially of:
about 150 mg/mL of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14;
ii) a second part that binds to IL-13 comprising a second heavy chain (HC2) comprising SEQ ID NO: 58 and a second light chain (LC2) comprising SEQ ID NO: 28;
about 20 mM of a histidine buffer (e.g., L-His/L-His HCl buffer);
about 120 mM trehalose (e.g., trehalose dihydrate);
about 150 mM lysine (e.g., L-lysine) or a pharmaceutically acceptable salt thereof (e.g., L-lysine HCl);
about 5 mM methionine (e.g., L-methionine) or a pharmaceutically acceptable salt thereof, and;
about 0.04% (w/v) polysorbate 80 (PS80).

In some embodiments, the pharmaceutical, preferably liquid (e.g., aqueous) composition has a pH of 6.5, and comprises or consists essentially of:
150 mg/mL of a bispecific antibody or antigen-binding fragment thereof, wherein the bispecific antibody comprises:
i) a first part that binds to IL-18 comprising a first heavy chain (HCl) comprising SEQ ID NO: 57 and a first light chain (LC1) comprising SEQ ID NO: 14;
ii) a second part that binds to IL-13 comprising a second heavy chain (HC2) comprising SEQ ID NO: 58 and a second light chain (LC2) comprising SEQ ID NO: 28;
20 mM of a histidine buffer (e.g., L-His/L-His HCl buffer);
120 mM trehalose (e.g., trehalose dihydrate);
150 mM lysine (e.g., L-lysine) or a pharmaceutically acceptable salt thereof (e.g., L-lysine HCl);
5 mM methionine (e.g., L-methionine) or a pharmaceutically acceptable salt thereof, and;
0.04% (w/v) polysorbate 80 (PS80).

In some embodiments, the polysorbate 80 (PS80) has low peroxide content, e.g., has a peroxide content of about ≤2 meqO2/kg, about ≤1.9 meqO2/kg, about ≤1.8 meqO2/kg, about ≤ 1.7 meqO2/kg, about ≤1.6 meqO2/kg, about ≤1.5 meqO2/kg, about ≤1.4 meqO2/kg, about ≤1.3 meqO2/kg, about ≤1.2 meqO2/kg, about ≤1.1 meqO2/kg, about ≤1 meqO2/kg, about ≤0.9 meqO2/kg, about ≤0.8 meqO2/kg, about ≤0.7 meqO2/kg, about ≤0.6 meqO2/kg, about ≤0.5 meqO2/kg, about ≤0.4 meqO2/kg, about ≤0.3 meqO2/kg, about ≤0.2 meqO2/kg, or about ≤0.1 meqO2/kg.

### Manufacture of Multispecific Antibody and Antigen-Binding Fragments Thereof

The multispecific, e.g., bispecific, antibodies and antigen-binding fragments thereof comprised by the pharmaceutical, preferably liquid (e.g., aqueous), compositions described herein can be generated using a number of techniques available to the skilled person. As a non-limiting example, the antibodies and antigen-binding fragments thereof can be generated as described in WO2023/209568 or WO2018/057955. As another non-limiting example, the antibodies and antigen-binding fragments thereof can be generated recombinantly, by introducing nucleic acid molecules encoding them into expression vectors and expressing them in host cells.

In some embodiments, the first part and the second part are encoded by the same nucleic acid molecule or expression vector. In some embodiments, the first part and the second part are encoded by separate nucleic acid molecules or expression vectors. The nucleic acid molecules can be produced using a variety of techniques, for example by de novo solid-phase DNA synthesis, amplification and/or mutagenesis of any existing molecule using PCR, and the like.

Suitable expression vectors are discussed earlier herein. The skilled person understands that the choice of the expression vector will depend on the intended host cell for expression. Typically, an expression vector additionally comprises a promoter and other regulatory sequences, operably linked to the nucleic acid molecule encoding the first part and/or the second part. In some embodiments, the promoter is a constitutive or an inducible promoter. The expression vector may additionally comprise a nucleotide sequence encoding for a secretion signal, operably linked to the first part that and/or the second part. The antibodies or antigen-binding fragments thereof are typically recovered from cell culture medium as secreted polypeptides, but may also be recovered from host cell lysate when produced without secretion signals. If the antibodies or antigen-binding fragments thereof are membrane-bound, they can be released from the membrane using e.g., a suitable detergent solution (e.g., Triton-X 100).

The host cell can be, but is not limited to, a prokaryotic cell (e.g., a bacterium, for example E. coli), a eukaryotic cell (e.g., a mammalian cell), an insect cell, or a human cell. The host cell may correspond to a cell line. Suitable eukaryotic cell lines include, but are not limited to, Vero cells, HeLa cells, COS cells, CHO cells, HEK cells, BHK cells, and MDCKII cells. Other examples of suitable eukaryotic cell lines include, but are not limited to, COS-1 (ATCC No. CRL 1650), COS-7 (ATCC No. CRL 1651), BHK570 (ATCC No. CRL 10314), DHFR-CHO (see Urlaub et al., 1986, Somatic Cell Mol. Genet. 12, 555-556), mouse Sertoli cells, monkey kidney cells, African green monkey kidney cells (ATCC No. CRL 1587), canine kidney cells (ATCC CCL34), human lung cells (ATCC CCL 75), Hep G2 and myeloma or lymphoma cells (e.g., NSO; see U.S. Pat. No. 5,807,715), Sp2/0, YO cells. Suitable insect cells, include, but are not limited to, Sf9 cells. In some embodiments, the host cell is a HEK cell or a CHO cell.

Methods for introducing expression vectors comprising the nucleic acid molecules of interest can vary depending on the type of host cell. For example, calcium chloride transfection is commonly utilized for prokaryotic cells, whereas calcium phosphate treatment or electroporation may be used for other cellular hosts (see generally Sambrook, et al., Molecular Cloning: A Laboratory Manual (4th Edition), 2012, Cold Spring Harbor Laboratory Press, USA). Other methods include, e.g., electroporation, calcium phosphate treatment, liposome-mediated transformation, injection and microinjection, ballistic methods, virosomes, immunoliposomes, polycation: nucleic acid conjugates, naked DNA, artificial virions, fusion to the herpes virus structural protein VP22 (e.g., see Elliot and O'Hare, (1997) Cell 88:223), agent-enhanced uptake of DNA, and ex vivo transduction.

Multispecific, e.g., bispecific, antibodies and antigen-binding fragments thereof may be produced via co-expression of the first part- and second-part described herein using a single host cell. In some embodiments wherein the antibody or antigen-binding fragment thereof comprises a first light chain of the lambda type and a second light chain of the kappa type, co-expression in a single host cell may be particularly advantageous as chain mispairing can be minimized. Alternatively, multispecific, e.g., bispecific, antibodies may be produced using multiple host cells, e.g., a first host cell may express the first part and a second host cell may express the second part. In such methods, each host cell may comprise a nucleic acid molecule or expression vector encoding the first-and/or second-part. The host cells may be cultured in the same or different cultures. Production of the multispecific, e.g., bispecific, antibody or antigen-binding fragment thereof will generally involve culturing the host cell(s) under conditions sufficient (conducive) to express the first- and second-part followed by recovery of the first- and second-part. In some cases, the produced polypeptides are recovered and optionally purified from recombinant cell proteins or polypeptides to obtain preparations that are substantially homogeneous as to heteromultimer. As a first step, the culture medium or lysate is typically centrifuged to remove particulate cell debris. Alternatively, depth filtration followed by sterile filtration may be used. The produced antibodies or antigen-binding fragments thereof (or the first part and/or second part) can be conveniently purified e,g., by hydroxylapatite chromatography, gel electrophoresis, dialysis, or affinity chromatography (e.g., on a CaptureSelect CH1-XL resin (Thermofisher Scientific, WA, MA, USA)). Other techniques for protein purification such as fractionation on an ion-exchange column, ethanol precipitation, reverse phase HPLC, chromatography on silica, chromatography on heparin sepharose, chromatography on an anion or cation exchange resin (such as a polyaspartic acid column), chromatofocusing, SDS-PAGE, protein A-affinity chromatography, and ammonium sulfate precipitation are also available. Purity may be e.g., determined using size exclusion chromatography, e.g., on commercial columns like the BEH200 SEC column (Waters #186005225). Purity may also be e.g., determined using CE-SDS capillary electrophoresis, e.g., on commercial systems like the Beckman Coulter PA 800 system (Beckman Coulter, CA, USA).

### Therapeutic Applications

The skilled person understands that aspects and embodiments described herein relating to the pharmaceutical compositions described herein for use in therapeutic applications are also applicable to their use in therapeutic applications, to their use in the manufacture of medicaments, as well to therapeutic methods comprising their administration to a subject in need thereof.

Accordingly, in a further aspect, there is provided a method of treatment in a subject in need thereof, comprising administering a therapeutically effective amount of a pharmaceutical composition described herein to the subject. In a further aspect, there is provided a pharmaceutical composition described herein for use as a medicament. In a further aspect, there is provided the use of a pharmaceutical composition described herein for the manufacture of a medicament. In a further aspect, there is provided the use of pharmaceutical composition described herein as a medicament.

The therapeutic methods and uses described herein generally comprise administering to a subject in need thereof a therapeutically effective amount of the pharmaceutical composition. A preferred subject is a human.

In a further aspect, there is provided a method of preventing and/or treating an IL-18 and/or IL-13 related disease or disorder in a subject in need thereof, comprising administering a therapeutically effective amount of a pharmaceutical composition described herein to the subject.

In a further aspect, there is provided a pharmaceutical composition described herein for use in preventing and/or treating an IL-18 and/or IL-13 related disease or disorder in a subject in need thereof.

In a further aspect, there is provided a use of a pharmaceutical composition described herein for the manufacture of a medicament for preventing and/or treating an IL-18 and/or IL-13 related disease or disorder in a subject in need thereof.

In a further aspect, there is provided a use of a pharmaceutical composition described herein for preventing and/or treating an IL-18 and/or IL-13 related disease or disorder in a subject in need thereof.

The phrases “IL-18-related (or mediated) disease or disorder”, “IL-13-related (or mediated) disease or disorder”, and “IL-18-and IL-13 related (or mediated) disease or disorder” are meant to encompass all diseases and medical conditions in which IL-18 and/or IL-13 (as applicable) are involved, whether directly or indirectly, in the disease or medical condition including the causation, development, progress, persistence or pathology of the disease or condition. Accordingly, the terms include diseases, disorders, and other medical conditions associated with or characterized by aberrant IL-18 and/or IL-13 levels (as applicable) and/or treatable by reducing or suppressing IL-18-and/or IL-13-induced activity in target cells or tissues (as applicable). The diseases or disorders or conditions to be treated may in some cases be caused e.g., by IL-18 and/or IL-13 dysregulation, for example aberrant signaling and/or expression of IL-18 and/or IL-13.

In some embodiments, the IL-18 and/or IL-13 related disease or disorder is an inflammatory and/or immune disease or disorder. In some embodiments, the IL-18 and/or IL-13 related disease or disorder is a skin disease or disorder.

In preferred embodiments, the IL-18 and/or IL-13 related disease or disorder is atopic dermatitis or a related condition. In some embodiments, the atopic dermatitis is moderate-to-severe atopic dermatitis.

In some embodiments, the subject in need thereof is suffering from atopic dermatitis and has one or more of the following:
an Eczema Area and Severity Index (EASI) score of ≥12 or ≥16, e.g., ≥16,
an Investigator's Global Assessment (IGA) score of ≥3, and/or
a Body Surface Area (BSA) of ≥10%.

The pharmaceutical compositions described herein can be used in the treatment and/or prevention of a symptom associated with atopic dermatitis. Non-limiting examples of symptoms associated with atopic dermatitis that are treatable include, but are not limited to, pruritus (itch), dry skin, thickened skin, cracked skin, scaly skin, raw skin, skin sensitivity, swollen skin, scaly and dry eczematous lesions, erythema, induration/papulation, excoriations, and lichenification.

Administration of a pharmaceutical composition described herein can result in the alleviation (decrease) of a symptom associated with atopic dermatitis relative to baseline (prior to treatment), preferably selected from the group consisting of erythema, induration/papulation, excoriations, lichenification, pruritus (itch), dry skin, thickened skin, cracked skin, scaly skin, raw skin, skin sensitivity, swollen skin, scaly and dry eczematous lesions, and a combination thereof. In some embodiments, the alleviation is at least 5%, at least 10%, at least 15%, at least 20%, at least 25%, at least 30%, at least 35%, at least 40%, at least 45%, at least 50%, at least 60%, at least 70%, at least 80%, at least 90%, at least 100%, or more, relative to baseline (prior to treatment).

Thus, in a further aspect, there is provided a method of preventing and/or treating pruritus, preferably pruritus associated with atopic dermatitis, in a subject in need thereof, comprising administering to the subject a therapeutically effective amount of a pharmaceutical composition described herein. In a further aspect, there is provided a pharmaceutical composition described herein for use in preventing and/or treating pruritus, preferably pruritus associated with atopic dermatitis in a subject in need thereof. In a further aspect, there is provided the use of a pharmaceutical composition described herein for the manufacture of a medicament for the treatment and/or prevention of pruritus, preferably pruritus associated with atopic dermatitis, in a subject in need thereof. In a further aspect, there is provided the use of a pharmaceutical composition described herein for treating and/or preventing pruritus, preferably pruritus associated with atopic dermatitis. In some embodiments, a prophylactically effective amount is administered.

The methods and uses described herein may result in an improvement (relative to baseline (prior to treatment)) in an atopic dermatitis-associated biomarker (positive response) in the subject to which the pharmaceutical composition is administered, for example a decrease in the concentration (level) of the biomarker in the subject's tissues and/or bodily fluids (e.g., blood, serum, plasma, and the like). Thus, in some embodiments, administration of the pharmaceutical composition results in an improvement relative to baseline in an atopic dermatitis-associated biomarker selected from the group consisting of CCL17 (TARC), pSTAT6, IFNγ, IL-13 (bioactive), IL-18 (bioactive) (e.g., serum free IL-18), and IL-18BP (e.g., serum IL-18BP). In some embodiments, the improvement (relative to prior to treatment) in the atopic dermatitis-associated biomarker, e.g., the decrease in its levels in the subject (e.g., in its tissues, serum or blood), is at least 5%, at least 10%, at least 15%, at least 20%, at least 25%, at least 30%, at least 35%, at least 40%, at least 45%, at least 50%, at least 60%, at least 70%, at least 80%, at least 90%, at least 100%, or more.

In some embodiments, the related condition to atopic dermatitis is selected from the group consisting of conditions related to inflammation, such as allergy (e.g., skin allergy, allergy to certain foods, pollen, mold, dust mite, animals, etc.), asthma (such as allergic and non-allergic asthma), infectious disorders, skin infection, cutaneous infection, eczema herpeticum, erysipelas, cellulitis, extra-cutaneous infection, encephalitis, endocarditis, infectious arthropathy, enterocolitis, septicemia, respiratory infection, upper respiratory tract infection, lower respiratory tract infection, lung infection, cardiac infection, brain infection, bone infection, and gastrointestinal infection, increased colonization with Staphylococcus aureus in lesional and non-lesional skin, autoimmune disorders, respiratory disorders, neuropsychiatric disorders, musculoskeletal disorders, and cardiovascular disorders associated with atopic dermatitis.

In some embodiments, the methods and uses described herein decrease the dependence of the subject to which treatment is administered on treatment with a topical medication. In some embodiments, the subject has moderate-to-severe AD. In some embodiments, administration of a treatment described herein decreases the required dose of a topical medication, e.g., of a TCS, by at least 5%, at least 10%, at least 15%, at least 20%, at least 30%, at least 40%, at least 50% or more, as compared to the dose required in the absence of treatment with the pharmaceutical composition described herein.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Actual dose levels may be varied so as to obtain an amount which is effective to achieve the desired therapeutic response for a particular patient.

An exemplary non-limiting dose of the multispecific antibody or antigen-binding fragment thereof administered to a patient may be from about 0.01 mg/kg to about 100 mg/kg of the patient's body weight, e.g., from about 1 mg/kg to about 100 mg/kg of the patient's body weight. Additional non-limiting examples of doses are about from 0.0001 mg/kg to about 100 mg/kg of the patient's body weight, e.g., from about 1 mg/kg to about 5 mg/kg, from about 5 mg/kg to about 10 mg/kg of the patient's body weight. Unit dose of the multispecific antibody or antigen-binding fragment thereof of present disclosure may be, but is not limited to, from about 0.1 mg to 100 mg, e.g., from about 1 mg to 5 mg, from about 5 mg to about 10 mg, from about 10 mg to about 25 mg, from about 25 mg to about 50 mg, from about 50 mg to about 100 mg, from about 100 mg to about 150 mg. Additional non-limiting examples of unit doses are about 150 mg, about 300 mg, and about 600 mg of the multispecific antibody or antigen-binding fragment thereof.

The skilled person understands that the amount of the pharmaceutical composition to be administered to the subject will depend on the concentration of the multispecific antibody or antigen-binding fragment thereof, as well as the intended dose of the multispecific antibody or antigen-binding fragment thereof to be administer. As a non-limiting example, if the intended dose of the multispecific antibody or antigen-binding fragment thereof is about 300 mg and the pharmaceutical composition comprises about 150 mg/mL of the antibody or antigen-binding fragment thereof, then about 2 mL of the pharmaceutical composition should be administered. Similarly, if the intended dose is about 600 mg, then about 4 mL of a pharmaceutical composition comprising about 150 mg/mL of the multispecific antibody or antigen-binding fragment thereof should be administered.

The means of administration will vary depending on the intended administration route. As a non-limiting example, in the case of intravenous or subcutaneous administration, the pharmaceutical composition may be administered via injection, e.g., using a needle, an injection device, an injection pen, a vial and syringe, pre-filled syringe, an autoinjector, and the like.

When a series of doses are administered, these may, for example, be administered approximately daily (e.g., daily), approximately every week (e.g., every week), approximately every 2 weeks (e.g., every 2 weeks), approximately every 3 weeks (e.g., every 3 weeks), approximately every 4 weeks (e.g., every 4 weeks), approximately every month (e.g., every month), approximately every 8 weeks (e.g., every 8 weeks), approximately every 2 months (e.g., every 2 months), approximately every 3 months (e.g., every 3 months), or approximately every 6 months (e.g., every 6 months). The doses may, for example, be administered until disease progression or one or more symptoms of the disease are ameliorated, disease progression is halted and/or reversed, and the like, as determined by a treating physician. For example, from about two, three, four, up to about 17 or more doses may be administered.

In a further aspect, there is provided a method of inhibiting IL-18 and/or IL-13 activity, the method comprising the step of contacting an effective amount of a pharmaceutical composition described herein with a plurality of cells. Preferably, the cells are mammalian cells, more preferably human cells. In some embodiments, the method is an ex vivo or an in vitro method. In some embodiments, the pharmaceutical composition is administered to a human subject.

### Combination Therapies

The pharmaceutical compositions described herein can be used in combination with additional therapeutic agents and therapies for the treatment and/or prevention of diseases, disorders and conditions, such as the ones described earlier herein.

Administered “in combination”, in reference to an additional therapeutic agent, refers to two (or more) different treatments are delivered to the subject during the course of treatment. In some embodiments, the administration of one treatment is still occurring when the administration of the second begins, so that there is overlap in terms of administration. This can also be referred to herein as “simultaneous” or “concurrent” administration. In other embodiments, the administration of one treatment ends before the administration of the other treatment begins (“sequential” administration). In some embodiments of either case, the treatment is more effective because of the combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, administration is such that the reduction in a symptom, or other parameter related to the disorder is greater than what would be observed with one treatment delivered in the absence of the other. The effect of the two treatments can be partially additive, wholly additive, or greater than additive. The administration can be such that an effect of the first treatment delivered is still detectable when the second is administered.

The term “concurrently” is not limited to the administration of therapies (e.g., prophylactic or therapeutic agents) at exactly the same time, but rather it is meant that a pharmaceutical composition described herein is administered to a subject in a sequence and within a time interval such that the pharmaceutical composition of the disclosure can act together with the additional therapeutic agent(s) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route, for example described earlier herein.

A pharmaceutical composition of the present disclosure and the additional therapeutic agent(s) can be administered simultaneously. In cases of simultaneous administration, the additional therapeutic agent(s) can be comprised by the pharmaceutical composition (comprising the multispecific antibody or antigen-binding fragment thereof) or can be comprised by a different pharmaceutical composition. For sequential administration, the pharmaceutical composition comprising the multispecific antibody or antigen-binding fragment thereof can be administered first, and the additional therapeutic agent(s) can be administered second, or the order of administration can be reversed. The additional therapeutic agent(s) may be administered to a subject by the same or different routes of administration compared to the multispecific antibody or antigen-binding fragment thereof.

The additional therapeutic agent(s) of the combination therapies of the present disclosure can also be cyclically administered. Combination cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

When administered in combination, the pharmaceutical composition comprising the multispecific antibody or fragment thereof of present disclosure, and the additional therapeutic agent(s) (e.g., second or third agent), or all, can be administered in an amount or dose that is higher, lower or the same than the amount or dose of each agent used individually, e.g., as a monotherapy. In certain embodiments, the administered amount or dose of the multispecific antibody or antigen-binding fragment thereof as described herein, of the additional agent (e.g., second or third agent), or of all compounds, is lower (e.g., at least 20%, at least 30%, at least 40%, or at least 50%) than the amount or dose of each agent used individually, e.g., as a monotherapy. In other embodiments, the amount or dose of the multispecific antibody or antigen-binding fragment thereof, of the additional agent (e.g., second or third agent), or of all compounds, that results in a desired effect (e.g., treatment of an immune disease or an inflammatory disease or condition, e.g., of AD) is lower (e.g., at least 20%, at least 30%, at least 40%, or at least 50% lower) than the amount or dose of each agent used individually, e.g., as a monotherapy, required to achieve the same therapeutic effect.

In some embodiments, the additional therapeutic agent (e.g., second or third agent) is an AD-therapeutic agent, e.g., small molecule, biological therapy, or an agent employing AD-therapeutic modality, e.g., phototherapy, including topical therapy, systemic therapy, phototherapy, and combinations thereof. “AD agent” includes topical therapies in the form of creams, ointments, lotions, gels or sprays (e.g., low-medium potency corticosteroids [Group IV-VII according to WHO guidelines, see Bolognia J L, Jorizzo J L, Schaffer J V. Glucocorticosteroids. Dermatology. 3rd ed. 2012. Ch 125, 2075-88; Ference J D, Last A R. Choosing topical corticosteroids. Am Fam Physician. 2009 Jan. 15; 79 (2): 135-40]); over the counter (OTC) emollients, and medical devices or so-called barrier creams (such as atopiclair); and lubricants for the treatment of itch and/or pain, e.g. anti-itch lotions containing menthol, pramoxine or anti-histamines; local anesthetics, systemic agents (e.g., biological agents, e.g., IL-4R inhibitors, for example dupilumab; IL-13Ra1 inhibitors, for example ASLAN-004; IL-13Ra2 inhibitors; IL-13 inhibitors, for example lebrikizumab, tralokinumab; IL-31 inhibitors, for example nemolizumab; TNF alpha inhibitors, for example adalimumab, infliximab, certolizumab and etanercept, alefacept; IL-la inhibitors, for example bermekimab (MABp1); IL-23 inhibitors, for example briakinumab, ustekinumab, guselkumab, risankizumab, tildrakizumab; IL-17 inhibitors, for example brodalumab, ixekizumab; CD11a inhibitors, for example efalizumab; IL-22 inhibitors, for example fezalimumab, IL-22 binding proteins; IL-5 inhibitors, for example mepolizumab, benralizumab; a synthetic form of IL-2, such as aldesleukin; recombinant IL-2 approaches targeting the interleukin-2 receptor complex, for example LY3471851; OSMR inhibitors, for example KPL-716; VAP-1 inhibitors; OX-40 inhibitors or OX40L inhibitors such as GBR830, KY1005; 1gE inhibitors, for example omalizumab, ligelizumab; TSLP inhibitors, for example tezepelumab; IL-33 inhibitors, for example MED13506; IL-36 inhibitors, for example spesolimab, ANB019; B-cell modulating approaches, for example rituximab, ocrelizumab; non-biological immunomodulating treatments, e.g., cyclosporine and other calcineurin inhibitors, JAK inhibitors, for example tofacitinib, upadacitinib, abrocitinib, baricitinib; TYK2 inhibitors, for example deucravacitinib; methotrexate; PDE4 inhibitors, for example apremilast; Siglec inhibitors, for example AK-002; S1P agonists or antagonists, for example etrasimod or SCD-044; BTK inhibitors, for example TAS-5315, IRAK4 antagonists and CCR4-inhibiting approaches, for example RPT-193; systemic corticosteroids, cyclophosphamide, sulphasalazine, azathioprin, mycophenolate mofetil, dapson, hydroxychloroquine; retinoids (e.g., alitretinoin); leukotriene inhibitors or antileukotrienes, for example, montelukast, pranlukast or zafirlukast, as well as 5-LO inhibitors, for example zileuton, and LTA4H inhibitors, for example acebilustat, intralesional corticosteroid injections; phototherapy (e.g. UVB and UVA high dose). photochemotherapy (e.g. psoralen and UVA (PUVA)); topical calcineurin inhibitors (cyclosporine, tacrolimus, pimecrolimus) or topical PDE4 inhibitors, for example crisaborole, difamilast or roflumilast; topical JAK inhibitors, for example ruxolitinib, delgocitinib, or topical Vitamin D analogues and topical aryl hydrocarbon receptor (AhR) inhibitors, for example benvitimod/tapinarof; topical corticosteroids of high—ultrahigh potency (Group I, II, Ill as per WHO definition); anti-fungal drugs with known anti-inflammatory properties, e.g., griseofulvin, itraconazole, betamethasone, dexamethasone, INCB018424, triamcinolone, apremilast, turmeric past, glucosamine sulfate, triamcinolone acetonide, sesame oil, betamethasone dipropionate, clobetasol propionate, probiotics (e.g., Bifidobacterium animalis subst. lactis HN019, Lactobacilli reuteri), omega-3, prednisone, prednisolone, platelet rich plasma, orabase paste, lycopene, topical chamomile, green tea, CO2 laser treatment, allergen specific immunotherapies, polybiotics, photobiomodulation, metronidazole, doxycycline, minocycline, cedar honey, purslane, curcuminoids, alefacept, hexaminolevulinate, hydroxychloroquine, adcortyl, efalizumab, fluocinolone, co-enzyme Q10 mucoadhesive tablets, chamaemelum nobile, sirolimus, tacrolimus, qingxuan decoction, NSAID topical rinse, NSAIDs, quercetin, NAVS naphthalan, valchlor, bupivacaine, oatmeal baths.

In some embodiments, the additional therapeutic agent is lebrikizumab (available as Ebglyss™). In some embodiments, the additional therapeutic agent is dupilumab (available as Dupixent®). In some embodiments, the additional therapeutic agent is nemolizumab (available as Nemluvio®). In some embodiments, the additional therapeutic agent is tralokinumab (available as Adtralza®).

The pharmaceutical compositions described herein may be used in combination with a topical medication. In some embodiments, the topical medication is selected from the group consisting of a topical steroid, e.g., corticosteroid, calcineurin inhibitor, e.g., topical calcineurin inhibitor, tacrolimus, pimecrolimus, a phosphodiesterase 4 (PDE4) inhibitor, e.g., topical PDE4 inhibitor, e.g. Crisaborole, an adrenocorticotropic hormone analog, and an aryl hydrocarbon receptor (AHR) agonist. In some embodiments, the topical corticosteroid is selected from the group consisting of methylprednisolone aceponate, mometasone furoate, fluticasone propionate, betamethasone valerate, triamcinolone, and hydrocortisone butyrate.

### Pharmaceutical Products

In a further aspect, there is provided a pharmaceutical product comprising a container comprising a pharmaceutical, preferably liquid (e.g., aqueous), composition described herein. In some embodiments, the container is selected from the group consisting of a vial, an injection device, an injection pen, a vial and syringe, a cartridge, a pre-filled syringe, and an autoinjector. In some embodiments, the container comprises from about 1 mL to about 2 mL injectable volume of the pharmaceutical composition. It is understood that the “fill” volume of the pharmaceutical composition in the container may be higher than the injectable volume, e.g., the fill volume for an injectable volume of about 1 mL may e.g., be about 1.1 mL, about 1.2 mL, and the like. In some embodiments, the fill volume is about 5%, about 10%, about 15%, or about 20% more than the injectable volume. In some embodiments, the container comprises about 1 mL (e.g., 1 mL) or about 2 mL (e.g., 2 mL) injectable volume of the pharmaceutical composition.

In a further aspect, there is provided a kit comprising a pharmaceutical, preferably liquid (e.g., aqueous), composition or a pharmaceutical product described herein, optionally further comprising instructions for use. The kit may comprise one or more doses of the pharmaceutical composition. Additionally, the kit may comprise means for administering the pharmaceutical composition. The phrase “means for administering” is used to indicate any available implement for systemically administering a drug to a patient. With such items, a patient may self-administer the drug (i.e., administer the drug without the assistance of a physician) or a medical practitioner may administer the drug. The instructions for use may provide the dose, route of administration, regimen, and total treatment duration for use with the comprised pharmaceutical composition or product. In some embodiments, a kit further comprises an additional therapeutic agent, e.g., an additional therapeutic agent described earlier herein.

The pharmaceutical compositions described herein may be in liquid (e.g., aqueous) form, or they may be a lyophilisate, that can e.g., be reconstituted shortly before use. It is thus understood that a liquid (e.g., aqueous) composition also encompasses reconstituted lyophilisates. In the case of reconstituted lyophilisates, the skilled person understands that reconstitution (e.g., with water) of the pharmaceutical composition should result in a liquid pharmaceutical composition comprising the multispecific antibody or antigen-binding fragment thereof and any pharmaceutically acceptable ingredient discussed earlier herein at the concentrations described earlier herein. Techniques for lyophilisation of antibodies are available in the art, e.g., see Rey & May (2004) Freeze-Drying/Lyophilization of Pharmaceutical & Biological Products ISBN 0824748689. Accordingly, in some embodiments, the pharmaceutical composition is a lyophilisate. In a further aspect, there is provided a lyophilisate obtained by or obtainable by lyophilisation of a liquid pharmaceutical composition described herein.

### Enumerated Embodiments

The following enumerated paragraphs correspond to additional enumerated embodiments pertaining to the disclosure.

Embodiment 1. A pharmaceutical composition comprising a multispecific antibody, wherein the multispecific antibody comprises:
i) a first part that binds to IL-18 comprising:
an HCDR1 comprising SEQ ID NO: 32;
an HCDR2 comprising SEQ ID NO: 33;
an HCDR3 comprising SEQ ID NO: 34;
an LCDR1 comprising SEQ ID NO: 4;
an LCDR2 comprising SEQ ID NO: 5, and;
an LCDR3 comprising SEQ ID NO: 6, and;
ii) a second part that binds to IL-13 comprising:
an HCDR1 comprising SEQ ID NO: 46;
an HCDR2 comprising SEQ ID NO: 47;
an HCDR3 comprising SEQ ID NO: 48;
an LCDR1 comprising SEQ ID NO: 18;
an LCDR2 comprising SEQ ID NO: 19, and;
an LCDR3 comprising SEQ ID NO: 20,
and wherein the pharmaceutical composition comprises from about 15 mg/mL to about 200 mg/mL of the multispecific antibody.

Embodiment 2. The pharmaceutical composition according to Embodiment 1, wherein the pharmaceutical composition is a liquid pharmaceutical composition, e.g., an aqueous pharmaceutical composition.

Embodiment 3. The pharmaceutical composition according to Embodiment 1 or 2, wherein the multispecific antibody is bispecific.

Embodiment 4. The pharmaceutical composition according to any one of Embodiments 1 to 3, wherein the pharmaceutical composition has a viscosity of about ≤20 cP, e.g., about ≤15 cP, measured at a temperature of about 25° C.

Embodiment 5. The pharmaceutical composition according to any one of Embodiments 1 to 4, wherein the pharmaceutical composition comprises from about 100 mM to about 250 mM of a viscosity reducing agent.

Embodiment 6. The pharmaceutical composition according to any one of Embodiments 1 to 5, wherein the viscosity reducing agent is selected from the group consisting of lysine, arginine, glycinamide hydrochloride, MgCl₂, NaCl, and a combination thereof.

Embodiment 7. The pharmaceutical composition according to any one of Embodiments 1 to 6, wherein the pharmaceutical composition comprises arginine (e.g., L-arginine) or lysine (e.g., L-lysine), or a pharmaceutically acceptable salt thereof.

Embodiment 8. The pharmaceutical composition according to any one of Embodiments 1 to 7, wherein the pharmaceutical composition comprises from about 100 mM to about 175 mM arginine (e.g., L-arginine) or lysine (e.g., L-lysine), or a pharmaceutically acceptable salt thereof.

Embodiment 9. The pharmaceutical composition according to any one of Embodiments 1 to 8, wherein the pharmaceutical composition comprises about 150 mM arginine (e.g., L-arginine) or lysine (e.g., L-lysine), preferably about 150 mM lysine (e.g., L-lysine).

Embodiment 10. The pharmaceutical composition according to any one of Embodiments 6 to 9, wherein the lysine is L-lysine or a pharmaceutically acceptable salt thereof, optionally L-lysine HCl.

Embodiment 11. The pharmaceutical composition according to any one of Embodiments 1 to 10, wherein the pharmaceutical composition has a pH of from about 5 to about 8.

Embodiment 12. The pharmaceutical composition according to any one of Embodiments 1 to 11, wherein the pharmaceutical composition has a pH of from about 5.5 to about 7.4.

Embodiment 13. The pharmaceutical composition according to any one of Embodiments 1 to 12, wherein the pharmaceutical composition has a pH of from about 6 to about 7.

Embodiment 14. The pharmaceutical composition according to any one of Embodiments 1 to 13, wherein the pharmaceutical composition has a pH of about 6.5.

Embodiment 15. The pharmaceutical composition according to any one of Embodiments 1 to 14, wherein the pharmaceutical composition comprises from about 50 mg/mL to about 175 mg/mL, from about 75 mg/mL to about 175 mg/mL, or from about 100 mg/mL to about 175 mg/mL of the multispecific antibody.

Embodiment 16. The pharmaceutical composition according to any one of Embodiments 1 to 15, wherein the pharmaceutical composition comprises about 150 mg/mL of the multispecific antibody.

Embodiment 17. The pharmaceutical composition according to any one of Embodiments 1 to 16, wherein the pharmaceutical composition comprises methionine or a pharmaceutically acceptable salt thereof.

Embodiment 18. The pharmaceutical composition according any one of Embodiments 1 to 17, wherein the pharmaceutical composition comprises from about 2.5 mM to about 20 mM methionine or a pharmaceutically acceptable salt thereof.

Embodiment 19. The pharmaceutical composition according to any one of Embodiments 1 to 18, wherein the pharmaceutical composition comprises about 5 mM methionine or a pharmaceutically acceptable salt thereof.

Embodiment 20. The pharmaceutical composition according to any one of Embodiments 17 to 19, wherein the methionine is L-methionine or a pharmaceutically acceptable salt thereof. Embodiment 21. The pharmaceutical composition according to any one of Embodiments 1 to 20, wherein the pharmaceutical composition comprises a sugar stabilizer.

Embodiment 22. The pharmaceutical composition according to Embodiment 21, wherein the pharmaceutical composition comprises from about 50 mM to about 220 mM of the sugar stabilizer.

Embodiment 23. The pharmaceutical composition according to Embodiments 21 or 22, wherein the sugar stabilizer is trehalose or sucrose.

Embodiment 24. The pharmaceutical composition according to any one of Embodiments 1 to 23, wherein the pharmaceutical composition comprises from about 100 mM to about 150 mM trehalose or sucrose, e.g., about 120 mM trehalose or sucrose.

Embodiment 25. The pharmaceutical composition according to any one of Embodiments 1 to 24, wherein the pharmaceutical composition comprises about 120 mM trehalose.

Embodiment 26. The pharmaceutical composition according to any one of Embodiments 23 to 25, wherein the trehalose is trehalose dihydrate.

Embodiment 27. The pharmaceutical composition according to any one of Embodiments 1 to 26, wherein the pharmaceutical composition comprises a buffering agent.

Embodiment 28. The pharmaceutical composition according to Embodiment 27, wherein the pharmaceutical composition comprises from about 5 mM to about 50 mM of the buffering agent.

Embodiment 29. The pharmaceutical composition according to Embodiment 27 or 28, wherein the buffering agent is selected from the group consisting of an acetate buffer, a citrate buffer, a phosphate buffer, a Tris buffer, a histidine buffer, and a combination thereof.

Embodiment 30. The pharmaceutical composition according to any one of Embodiments 1 to 29, wherein the pharmaceutical composition comprises from about 10 mM to about 25 mM of a histidine buffer, preferably about 20 mM of a L-histidine buffer.

Embodiment 31. The pharmaceutical composition according to Embodiment 29 or 30, wherein the histidine buffer is an L-His/L-His HCl buffer.

Embodiment 32. The pharmaceutical composition according to any one of Embodiments 1 to 31, wherein the pharmaceutical composition comprises about 20 mM L-His/L-His HCl buffer.

Embodiment 33. The pharmaceutical composition according to any one of Embodiments 1 to 32, wherein the pharmaceutical composition comprises a surfactant.

Embodiment 34. The pharmaceutical composition according to Embodiment 33, wherein the pharmaceutical composition comprises from about 0.01% (w/v) to about 0.1% (w/v) of the surfactant.

Embodiment 35. The pharmaceutical composition according to Embodiment 33 or 34, wherein the surfactant is selected from the group consisting of polysorbate 80 (PS80), polysorbate 20 (PS20), Polox188, and a combination thereof.

Embodiment 36. The pharmaceutical composition according to any one of Embodiments 1 to 35, wherein the pharmaceutical composition comprises from about 0.02% (w/v) to 0.08% (w/v) polysorbate 80 (PS80) or polysorbate 20 (PS20).

Embodiment 37. The pharmaceutical composition according to any one of Embodiments 1 to 36, wherein the pharmaceutical composition comprises about 0.04% (w/v) polysorbate 80 (PS80).

Embodiment 38. The pharmaceutical composition according to any one of Embodiments 1 to 37, wherein:
the first part that binds to IL-18 comprises a first variable heavy region (VH1) comprising SEQ ID NO: 41 and a first variable light region (VL1) comprising SEQ ID NO: 13, and;
the second part that binds to IL-13 comprises a second variable heavy region (VH2) comprising SEQ ID NO: 55 and a second variable light region (VL2) comprising SEQ ID NO: 27.

Embodiment 39. The pharmaceutical composition according to any one of Embodiments 1 to 38, wherein the first part that binds to IL-18 comprises a first light chain (LC1) that is a lambda light chain and the second part that binds to IL-13 comprises a second light chain (LC2) that is a kappa light chain.

Embodiment 40. The pharmaceutical composition according to any one of Embodiments 1 to 39, wherein the first part that binds to IL-18 comprises a first light chain (LC1) comprising SEQ ID NO: 14 and the second part that binds to IL-13 comprises a second light chain (LC2) comprising SEQ ID NO: 28.

Embodiment 41. The pharmaceutical composition according to any one of Embodiments 1 to 40, wherein:
the first part that binds to IL-18 comprises a first Fc polypeptide, and
the second part that binds to IL-13 comprises a second Fc polypeptide, wherein the first and second Fc polypeptides are associated forming an Fc region.

Embodiment 42. The pharmaceutical composition according to Embodiment 41, wherein the first and/or second Fc polypeptide comprises the amino acid sequence SEQ ID NO: 59 or a variant thereof.

Embodiment 43. The pharmaceutical composition according to Embodiment 41 or 42, wherein the first and/or second Fc polypeptide comprises a variant of SEQ ID NO: 59 comprising one or more amino acid substitutions selected from the group consisting of:
a. one or more half-life extension substitutions, e.g., an M252Y, an S254T, and a T256E substitution (“YTE”), and;
b. one or more K-i-H substitutions, e.g., an S354C and/or a T366W substitution or a Y349C, a T366S, an L368A, and/or a Y407V substitution, according to EU numbering.

Embodiment 44. The pharmaceutical composition according to any one of Embodiments 41 to 43, wherein:
the first part that binds to IL-18 comprises a first Fc polypeptide comprising a variant of SEQ ID NO: 59 comprising:
a) an M252Y, an S254T, and a T256E substitution (“YTE”), and;
b) a Y349C, a T366S, an L368A, and a Y407V substitution, and;
the second part that binds to IL-13 comprises a second Fc polypeptide comprising a variant of SEQ ID NO: 59 comprising:
c) an M252Y, an S254T, and a T256E substitution (“YTE”), and;
d) an S354C and a T366W substitution, or
the first part that binds to IL-18 comprises a first Fc polypeptide comprising a variant of SEQ ID NO: 59 comprising:
a) an M252Y, an S254T, and a T256E substitution (“YTE”), and;
b) an S354C and a T366W substitution, and;
the second part that binds to IL-13 comprises a second Fc polypeptide comprising a variant of SEQ ID NO: 59 comprising:
c) an M252Y, an S254T, and a T256E substitution (“YTE”), and;
d) a Y349C, a T366S, an L368A, and a Y407V substitution, according to EU numbering.

Embodiment 45. The pharmaceutical composition according to any one of Embodiments 1 to 44, wherein the multispecific antibody is bispecific and comprises:
i) a first part that binds to IL-18 comprising a first heavy chain (HCl) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28.

Embodiment 46. The pharmaceutical composition according to any one of Embodiments 1 to 45, wherein the multispecific antibody is bispecific and comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28.

Embodiment 47. The pharmaceutical composition according to any one of Embodiments 1 to 46, wherein the IL-18 and IL-13 are human.

Embodiment 48. The pharmaceutical composition according to any one of Embodiments 1 to 47, wherein the pharmaceutical composition is a stable pharmaceutical composition, preferably a stable liquid pharmaceutical composition.

Embodiment 49. The pharmaceutical composition according to Embodiment 48, wherein the pharmaceutical composition is stable at a temperature of about 5° C. for at least 6 months.

Embodiment 50. The pharmaceutical composition according to Embodiment 48 or 49, wherein the pharmaceutical composition is stable at a temperature of about 25° C. for at least 3 months.

Embodiment 51. The pharmaceutical composition according to any one of Embodiments 48 to 50, wherein the pharmaceutical composition is stable at a temperature of about 40° C. for at least 4 weeks.

Embodiment 52. The pharmaceutical composition according to any one of Embodiments 1 to 51, wherein the pharmaceutical composition is suitable for subcutaneous administration.

Embodiment 53. The pharmaceutical composition according to any one of Embodiments 1 to 52, wherein the pharmaceutical composition has a pH of from about 5 to about 8, wherein the multispecific antibody is bispecific and comprises:
i) a first part that binds to IL-18 comprising a first heavy chain (HCl) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28, and wherein the pharmaceutical composition is a liquid pharmaceutical composition comprising:
from about 100 mg/mL to about 175 mg/mL of the multispecific antibody;
from about 100 mM to about 175 mM lysine (e.g., L-lysine) or a pharmaceutically acceptable salt thereof (e.g., L-lysine HCl).

Embodiment 54. The pharmaceutical composition according to any one of Embodiments 1 to 52, wherein the pharmaceutical composition has a pH of from about 5 to about 8, wherein the multispecific antibody is bispecific and comprises:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) comprising SEQ ID NO: 58 and a second light chain (LC2) comprising SEQ ID NO: 28, and wherein the pharmaceutical composition is a liquid pharmaceutical composition comprising:
from about 100 mg/mL to about 175 mg/mL of the multispecific antibody;
from about 100 mM to about 175 mM lysine (e.g., L-lysine) or a pharmaceutically acceptable salt thereof (e.g., L-lysine HCl).

Embodiment 55. The pharmaceutical composition according to Embodiment 53 or 54, wherein the pharmaceutical composition comprises about 150 mg/mL of the bispecific antibody.

Embodiment 56. The pharmaceutical composition according to any one of Embodiments 53 to 55, wherein the pharmaceutical composition comprises from about 2.5 mM to about 20 mM methionine (e.g., L-methionine) or a pharmaceutically acceptable salt thereof.

Embodiment 57. The pharmaceutical composition according to Embodiment 56, wherein the pharmaceutical composition comprises about 5 mM methionine (e.g., L-methionine) or a pharmaceutically acceptable salt thereof.

Embodiment 58. The pharmaceutical composition according to any one of Embodiments 53 to 57, wherein the pharmaceutical composition comprises from about 100 mM to about 150 mM trehalose (e.g., trehalose dihydrate).

Embodiment 59. The pharmaceutical composition according to Embodiment 58, wherein the pharmaceutical composition comprises about 120 mM trehalose (e.g., trehalose dihydrate).

Embodiment 60. The pharmaceutical composition according to any one of Embodiments 53 to 59, wherein the pharmaceutical composition comprises from about 10 mM to about 25 mM of a histidine buffer (e.g., L-His/L-His HCl buffer).

Embodiment 61. The pharmaceutical composition according to Embodiment 60, wherein the pharmaceutical composition comprises about 20 mM of a histidine buffer (e.g., L-His/L-His HCl buffer).

Embodiment 62. The pharmaceutical composition according to any one of Embodiments 53 to 61, wherein the pharmaceutical composition comprises from about 0.02% (w/v) to about 0.08% (w/v) polysorbate 80 (PS80).

Embodiment 63. The pharmaceutical composition according to any one of Embodiments 53 to 62, wherein the pharmaceutical composition comprises about 0.04% (w/v) polysorbate 80.

Embodiment 64. The pharmaceutical composition according to any one of Embodiments 53 to 63, wherein the pharmaceutical composition has a pH of about 6.5.

Embodiment 65. The pharmaceutical composition according to any one of Embodiments 53 or 55 to 64, wherein the pharmaceutical composition comprises:
about 150 mg/mL of a bispecific antibody comprising:
i) a first part that binds to IL-18 comprising a first heavy chain (HCl) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28;
about 20 mM of a histidine buffer, e.g., L-His/L-His HCl buffer;
about 120 mM trehalose, e.g., trehalose dihydrate;
about 150 mM lysine (e.g., L-lysine) or a pharmaceutically acceptable salt thereof (e.g., L-lysine HCl);
about 5 mM methionine (e.g., L-methionine) or a pharmaceutically acceptable salt thereof;
about 0.04% (w/v) polysorbate 80 (PS80),
and wherein the pharmaceutical composition has a pH of about 6.5.

Embodiment 66. The pharmaceutical composition according to any one of Embodiments 54 to 64, wherein the pharmaceutical composition comprises:

about 150 mg/mL of a bispecific antibody comprising:
i) a first part that binds to IL-18 comprising a first heavy chain (HCl) comprising SEQ ID NO: 57 and a first light chain (LC1) comprising SEQ ID NO: 14, and;
ii) a second part that binds to IL-13 comprising a second heavy chain (HC2) SEQ ID NO: 58 and a second light chain (LC2) comprising SEQ ID NO: 28;
about 20 mM of a histidine buffer, e.g., L-His/L-His HCl buffer;
about 120 mM trehalose, e.g., trehalose dihydrate;
about 150 mM lysine (e.g., L-lysine) or a pharmaceutically acceptable salt thereof (e.g., L-lysine HCl);
about 5 mM methionine (e.g., L-methionine) or a pharmaceutically acceptable salt thereof;
about 0.04% (w/v) polysorbate 80 (PS80),
and wherein the pharmaceutical composition has a pH of about 6.5.

Embodiment 67. A pharmaceutical product comprising a container comprising the pharmaceutical composition of any one of Embodiments 1 to 66, optionally wherein the container is selected from the group consisting of a vial, an injection device, an injection pen, a vial and syringe, a cartridge, a pre-filled syringe, and an autoinjector.

Embodiment 68. The pharmaceutical product according to Embodiment 67, wherein the container comprises from about 1 mL to about 2 mL, e.g., about 1 mL or about 2 mL, injectable volume of the pharmaceutical composition.

Embodiment 69. A kit comprising the pharmaceutical composition of any one of Embodiments 1 to 66, or the pharmaceutical product of Embodiment 67 or 68, optionally further comprising instructions for use.

Embodiment 70. A lyophilisate obtained by or obtainable by lyophilisation of the pharmaceutical composition of any one of Embodiments 2 to 66.

Embodiment 71. A method of preventing and/or treating an IL-18 and/or IL-13 related disease or disorder in a subject in need thereof, comprising administering a therapeutically effective amount of the pharmaceutical composition of any one of Embodiments 1 to 66 to the subject.

Embodiment 72. The method according to Embodiment 71, wherein the IL-18 and/or IL-13 related disease or disorder is an inflammatory and/or immune disease or disorder.

Embodiment 73. The method according to Embodiment 71 or 72, wherein the IL-18 and/or IL-13 related disease or disorder is a skin disease or disorder.

Embodiment 74. The method according to any one of Embodiments 71 to 73, wherein the IL-18 and/or IL-13 related disease or disorder is atopic dermatitis or a related condition.

Embodiment 75. The method according to Embodiment 74, wherein the atopic dermatitis is moderate-to-severe atopic dermatitis.

Embodiment 76. The method according to any one of Embodiments 71 to 75, wherein the subject is a human.

Embodiment 77. A pharmaceutical composition according to any one of Embodiments 1 to 66 for use in preventing and/or treating an IL-18 and/or IL-13 related disease or disorder in a subject in need thereof.

Embodiment 78. The pharmaceutical composition for use according to Embodiment 77, wherein the IL-18 and/or IL-13 related disease or disorder is an inflammatory and/or immune disease or disorder.

Embodiment 79. The pharmaceutical composition for use according to Embodiment 77 or 78, wherein the IL-18 and/or IL-13 related disease or disorder is a skin disease or disorder.

Embodiment 80. The pharmaceutical composition for use according to any one of Embodiments 77 to 79, wherein the IL-18 and/or IL-13 related disease or disorder is atopic dermatitis or a related condition.

Embodiment 81. The pharmaceutical composition for use according to Embodiment 80, wherein the atopic dermatitis is moderate-to-severe atopic dermatitis.

Embodiment 82. The pharmaceutical composition for use according to any one of Embodiments 77 to 81, wherein the subject is a human.

Embodiment 83. A use of a pharmaceutical composition according to any one of Embodiments 1 to 66 for the manufacture of a medicament for preventing and/or treating an IL-18 and/or IL-13 related disease or disorder in a subject in need thereof.

Embodiment 84. The use according to Embodiment 83, wherein the IL-18 and/or IL-13 related disease or disorder is an inflammatory and/or immune disease or disorder.

Embodiment 85. The use according to Embodiment 83 or 84, wherein the IL-18 and/or IL-13 related disease or disorder is a skin disease or disorder.

Embodiment 86. The use according to any one of Embodiments 83 to 85, wherein the IL-18 and/or IL-13 related disease or disorder is atopic dermatitis or a related condition.

Embodiment 87. The use according to Embodiment 86, wherein the atopic dermatitis is moderate-to-severe atopic dermatitis.

Embodiment 88. The use according to any one of Embodiments 83 to 87, wherein the subject is a human.

### General

The contents of any patents, patent applications, and references cited throughout this specification are hereby incorporated by reference in their entireties. Unless otherwise required by context, singular terms used herein shall include pluralities and plural terms shall include the singular.

Various embodiments of the invention are described herein. It will be recognized that features specified in each embodiment may be combined with other specified features to provide further embodiments of the present invention. When an embodiment is described as being “according to” or “of” a previous embodiment, the previous embodiment includes sub-embodiments thereof.

The present disclosure includes a sequence listing. Certain sequences represented by specific SEQ ID NOs are further included in the detailed description. In cases where a discrepancy arises between a sequence having a SEQ ID NO in the detailed description and a sequence having the same SEQ ID NO as disclosed in the sequence listing, the detailed description prevails.

The following examples are provided to further illustrate the disclosure but should not be considered to limit its scope in any way. Other variants of the disclosure will be readily apparent to one of ordinary skill in the art and are encompassed by the description and the appended claims.

### EXAMPLES

### Example 1. Buffer-pH-Excipient Biophysical Screening to Evaluate Conformational (Tₒₙₛₑₜ, Tₘ) and Colloidal (kD) Stability

In Example 1, the conformational (thermal) stability of an anti-IL-18/IL-13 bispecific antibody (anti-IL-18 sequence: SEQ ID NO: 41 (VH1), SEQ ID NO: 13 (VL1), SEQ ID NO: 42 (HCl), SEQ ID NO: 14 (LC1); anti-IL-13 sequence: SEQ ID NO: 55 (VH2), SEQ ID NO: 27 (VL2), SEQ ID NO: 56 (HC2), SEQ ID NO: 28 (LC2)) was assessed by its formulation in a pH range of from 5 to 8 using five different buffer types, with or without the presence of lysine and trehalose at constant concentration of 150 mM and 120 mM respectively. A total of 28 different formulations (1-F1 to 1-F28) were tested.

The kD (dissociation constant) represents the binding affinity between protein molecules and potentially destabilizing agents. A lower kD value can lead to stronger binding, potentially causing protein aggregation and viscosity perturbation. Understanding kD is crucial for assessing how binding interactions may impact colloidal stability and viscosity in protein formulations.

The melting temperature (Tₘ) is a critical indicator of conformation (thermal) stability in proteins. A higher Tₘ value typically indicates a more stable protein that requires a higher temperature to unfold. The onset temperature (Tₒₙₛₑₜ) is the temperature at which the protein begins to denature or unfold. It signifies the initial stages of protein unfolding before complete denaturation occurs. Monitoring Tₒₙₛₑₜ can provide insights into the protein's thermal behavior and stability under different conditions. Therefore, both Tₘ and Tₒₙₛₑₜ are valuable parameters in studying the thermal stability of proteins and assessing their resistance to unfolding.

The parameters of kD, describing the colloidal stability (in linear range of 2.5-10 mg/mL anti-IL18/IL-13 bispecific), and Tₘ and Tₒₙₛₑₜ, describing the conformational (thermal) stability (on 7.5 mg/mL and 10 mg/mL anti-IL18/IL-13 bispecific), were measured using the UNCLE instrument (Unchained Labs, CA, USA). The Uncle instrument is all-in-one platform for evaluating protein stability that uses 3 detection methods: full-spectrum fluorescence, static light scattering (SLS), and dynamic light scattering (DLS) to fully profile protein stability. All parameters were measured according to the manufacturer's instructions.

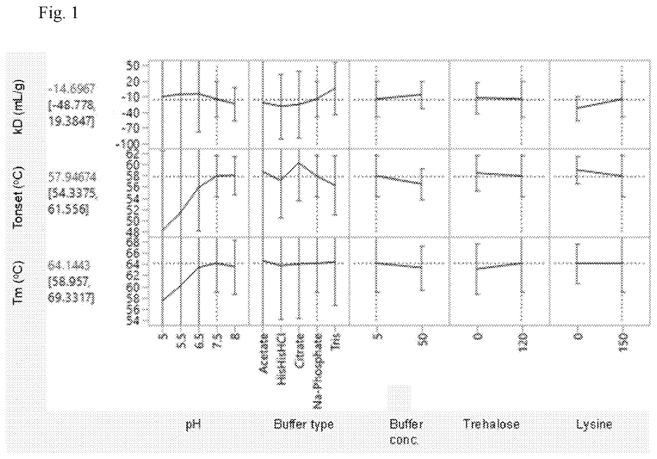

Results showed that pH value, buffer type, lysine concentration, and sugar concentration have an effect on colloidal and thermal stability. FIG. 1 shows a positive effect on thermal stability with increasing pH, while colloidal stability is the highest at pH 5.5 and 6.5. The presence of lysine had a positive effect on kD, enhancing colloidal stability.

TABLE 2
Formulations 1-F1 to 1-F28

| | | | | | Amino | |
|---|---|---|---|---|---|---|
| | mAb | | Buffer | | acid | Sugar |
| F# | [mg/mL] | Buffer type | [mM] | pH | [150 mM] | [120 mM] |
| 1-F1 | 10 | Na-Acetate | 5 | 5 | L-Lysine | — |
| | | | | | HCl | |
| 1-F2 | 10 | Na-Acetate | 50 | 5 | — | — |
| 1-F3 | 10 | Na-Acetate | 50 | 5.5 | L-Lysine | Trehalose |
| | | | | | HCl | dihydrate |
| 1-F4 | 10 | Na-Citrate | 50 | 5.5 | — | Trehalose |
| | | | | | | dihydrate |
| 1-F5 | 10 | Na-Citrate | 5 | 5.5 | — | — |
| 1-F6 | 10 | L-His/L-His | 5 | 5.5 | — | Trehalose |
| | | HCl | | | | dihydrate |
| 1-F7 | 10 | Na-Acetate | 5 | 5.5 | — | Trehalose |
| | | | | | | dihydrate |
| 1-F8 | 10 | L-His/L-His | 50 | 5.5 | L-Lysine | Trehalose |
| | | HCl | | | HCl | dihydrate |
| 1-F9 | 10 | L-His/L-His | 50 | 6.5 | L-Lysine | — |
| | | HCl | | | HCl | |
| 1-F10 | 10 | Na-Phosphate | 5 | 6.5 | — | Trehalose |
| | | | | | | dihydrate |
| 1-F11 | 10 | Na-Phosphate | 50 | 6.5 | L-Lysine | Trehalose |
| | | | | | HCl | dihydrate |
| 1-F12 | 10 | L-His/L-His | 50 | 6.5 | — | — |
| | | HCl | | | | |
| 1-F13 | 10 | L-His/L-His | 5 | 6.5 | L-Lysine | — |
| | | HCl | | | HCl | |
| 1-F14 | 10 | Na-Citrate | 5 | 6.5 | — | Trehalose |
| | | | | | | dihydrate |
| 1-F15 | 10 | Na-Citrate | 5 | 6.5 | L-Lysine | Trehalose |
| | | | | | HCl | dihydrate |
| 1-F16 | 10 | Na-Phosphate | 50 | 6.5 | — | — |
| 1-F17 | 10 | Tris-HCl | 5 | 7.5 | — | — |
| 1-F18 | 10 | Tris-HCl | 50 | 7.5 | L-Lysine | Trehalose |
| | | | | | HCl | dihydrate |
| 1-F19 | 10 | Na-Phosphate | 50 | 8 | — | Trehalose |
| | | | | | | dihydrate |
| 1-F20 | 10 | Na-Phosphate | 50 | 8 | L-Lysine | Trehalose |
| | | | | | HCl | dihydrate |
| 1-F21 | 10 | Na-Phosphate | 5 | 8 | L-Lysine | — |
| | | | | | HCl | |
| 1-F22 | 10 | Tris-HCl | 50 | 8 | — | Trehalose |
| | | | | | | dihydrate |
| 1-F23 | 10 | Na-Phosphate | 5 | 8 | — | Trehalose |
| | | | | | | dihydrate |
| 1-F24 | 10 | Tris-HCl | 5 | 8 | L-Lysine | — |
| | | | | | HCl | |
| 1-F25 | 10 | Na-Phosphate | 5 | 7.5 | — | Trehalose |
| | | | | | | dihydrate |
| 1-F26 | 10 | Na-Phosphate | 50 | 7.5 | — | Trehalose |
| | | | | | | dihydrate |
| 1-F27 | 10 | Na-Phosphate | 50 | 7.5 | L-Lysine | — |
| | | | | | HCl | |
| 1-F28 | 10 | Na-Phosphate | 5 | 7.5 | L-Lysine | Trehalose |
| | | | | | HCl | dihydrate |

### Example 2. Impact of Different Formulation Ingredients on Viscosity and Conformational (Tₒₙₛₑₜ, Tₘ) Stability

In Example 2, the effect of different anti-IL-18/IL-13 bispecific antibody (anti-IL-18 sequence: SEQ ID NO: 41 (VH1), SEQ ID NO: 13 (VL1), SEQ ID NO: 42 (HCl), SEQ ID NO: 14 (LC1); anti-IL-13 sequence: SEQ ID NO: 55 (VH2), SEQ ID NO: 27 (VL2), SEQ ID NO: 56 (HC2), SEQ ID NO: 28 (LC2)) concentrations, pH, buffer types, sugars, and viscosity reducing agents (VRAs) on formulation viscosity and conformational (thermal) stability was evaluated. Thermal stability was assessed using an UNCLE instrument (Unchained Labs, CA, USA) as described in Example 1. Viscosity measurements were performed using an Anton-Paar rheometer (Anton-Paar, Graz, AT). The experiment was conducted at a constant temperature of 25° C. to maintain the consistency of measurements. Dynamic viscosity was determined at a shear rate of 1000 s⁻¹.

The bispecific antibody was formulated in formulations spanning a pH range from 5.5 to 7 using different buffer types, with or without two different amino acids (lysine or arginine) and/or NaCl, in the presence of trehalose or sucrose. A total of 16 different formulations (2-F1 to 2-F16) were tested.

TABLE 3
Formulations 2-F1 to 2-F16

| | mAb | Buffer type | | Amino acid | NaCl | Sugar |
|---|---|---|---|---|---|---|
| F# | [mg/mL] | [20 mM] | pH | [mM] | [mM] | [mM] |
| 2-F1 | 150 | L-His/L-His | 7 | L-Lysine | — | Trehalose |
| | | HCl | | HCl 150 mM | | dihydrate |
| | | | | | | 220 mM |
| 2-F2 | 153 | L-His/L-His | 7 | L-Lysine | — | Trehalose |
| | | HCl | | HCl 100 mM | | dihydrate |
| | | | | | | 220 mM |
| 2-F3 | 150 | Na-Citrate | 7 | L-Lysine | — | Trehalose |
| | | | | HCl 150 mM | | dihydrate |
| | | | | | | 220 mM |
| 2-F4 | 136 | L-His/L-His | 7 | L-Lysine | — | Trehalose |
| | | HCl | | HCl 200 mM | | dihydrate |
| | | | | | | 220 mM |
| 2-F5 | 140 | Na-Citrate | 6.5 | — | 100 | Trehalose |
| | | | | | | dihydrate |
| | | | | | | 220 mM |
| 2-F6 | 140 | Na-Phosphate | 7 | L-Lysine | — | Trehalose |
| | | | | HCl 150 mM | | dihydrate |
| | | | | | | 220 mM |
| 2-F7 | 119 | L-His/L-His | 7 | L-Lysine | 100 | Trehalose |
| | | HCl | | HCl 150 mM | | dihydrate |
| | | | | | | 220 mM |
| 2-F8 | 128 | Na-Citrate | 6.5 | L-Lysine | — | Trehalose |
| | | | | HCl 150 mM | | dihydrate |
| | | | | | | 220 mM |
| 2-F9 | 121 | L-His/L-His | 7 | L-Lysine | — | Trehalose |
| | | HCl | | HCl 150 mM | | dihydrate |
| | | | | | | 120 mM |
| 2-F10 | 107 | Na-Citrate | 5.5 | L-Lysine | — | Trehalose |
| | | | | HCl 150 mM | | dihydrate |
| | | | | | | 220 mM |
| 2-F11 | 108 | L-His/L-His | 5.5 | — | 100 | Trehalose |
| | | HCl | | | | dihydrate |
| | | | | | | 220 mM |
| 2-F12 | 105 | L-His/L-His | 7 | — | 100 | Trehalose |
| | | HCl | | | | dihydrate |
| | | | | | | 220 mM |
| 2-F13 | 131 | L-His/L-His | 7 | L-Lysine | — | Sucrose |
| | | HCl | | HCl 150 mM | | 120 mM |
| 2-F14 | 150 | L-His/L-His | 7 | L-Arginine | — | Sucrose |
| | | HCl | | HCl 150 mM | | 120 mM |
| 2-F15 | 150 | L-His/L-His | 5.5 | L-Arginine | — | Sucrose |
| | | HCl | | HCl 150 mM | | 120 mM |
| 2-F16 | 137 | L-His/L-His | 5.5 | — | 100 | Sucrose |
| | | HCl | | | | 120 mM |

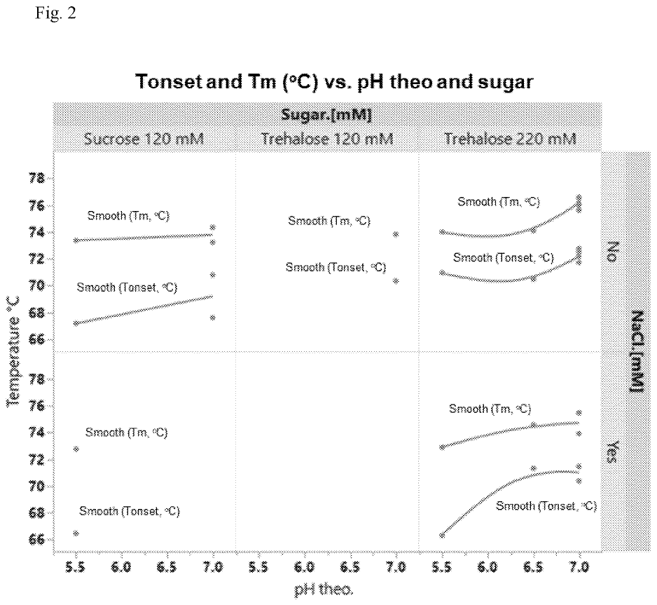

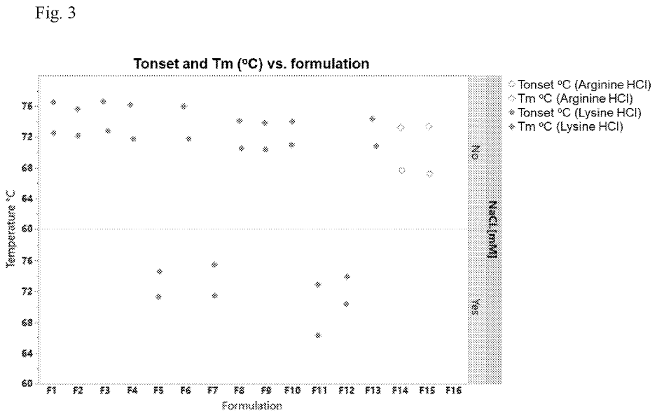

Tₒₙₛₑₜ and Tₘ showed higher temperature values in formulations with both higher pH values and trehalose concentrations (FIG. 2). Analytical results indicate a most pronounced positive effect concerning stabilization in the presence of lysine, compared to arginine (FIG. 3). Moreover, NaCl appears to affect both Tₘ and Tₒₙₛₑₜ in formulations comprising it, as they consistently exhibit lower values. There was no notable difference regarding various buffer types on thermal stability (FIG. 4).

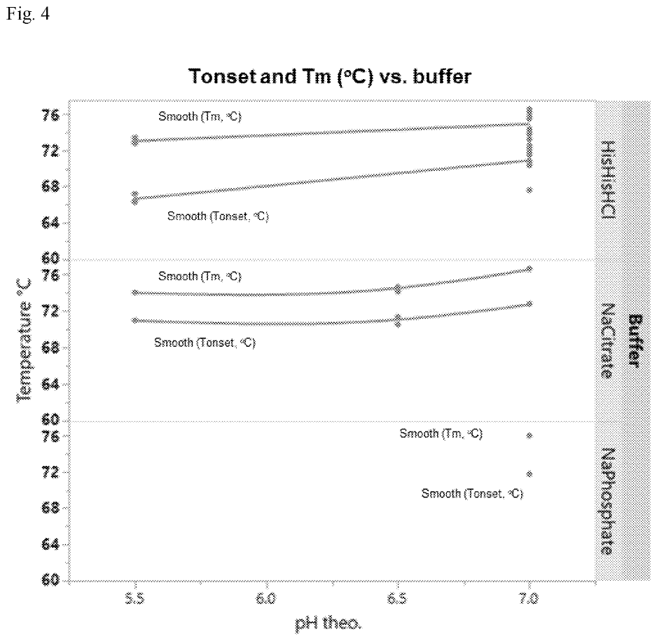

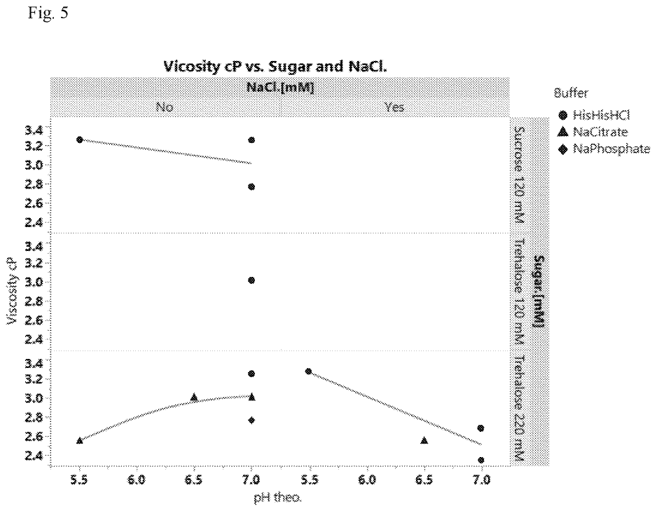

A trend towards reduced viscosity was obtained for formulations comprising 220 mM trehalose in presence of NaCl in histidine buffer with increasing pH (FIG. 5). Further, the addition of lysine showed a positive effect on reducing viscosity, particularly at higher pH values (FIG. 6).

### Example 3. Impact of Different Formulation Ingredients on Viscosity and Conformational (Tₒₙₛₑₜ, Tₘ) Stability in 20 mM Histidine pH 5 Buffer

In Example 3, the effect of different viscosity reducing agents (VRAs) on viscosity and conformational (thermal) stability of formulations comprising the bispecific antibody of Examples 1 and 2 was evaluated utilizing the same procedures, while viscosity was analyzed by dynamic light scattering (DLS) with an UNCLE instrument (Unchained Labs, CA, USA), by rearranging the Stokes-Einstein equation (He et al. (2010), Anal Biochem 399 (1): 141-143): Viscosity=kT/6 πDR_{H}, where D is the diffusion coefficient, k is the Boltzmann constant, T is the temperature, and R_{H} is the radius of the spherical diffusant. At a given temperature, the diffusion coefficient of a given particle depends only on its size and on the viscosity of the solution. The UNCLE method employs standard polystyrene beads as references particles with a known size, and uses DLS to measure their “apparent” size in a protein solution with unknown viscosity (prior to measurement). Viscosity measurements were performed at room temperature.

The bispecific antibody was formulated in formulations comprising 25 mg/mL antibody (thermal stability analysis) or 50 mg/mL (viscosity measurements), with L-His/L-His HCl as the buffer at pH 5, and with different viscosity reducing agents. A total of 8 different formulations (3-F1 to 3-F8) were tested, with 3-F1 (minimally formulated) serving as the reference formulation.

TABLE 4
Formulations 3-F1 to 3-F8

| | mAb | Buffer type | | Amino acid | Salt |
|---|---|---|---|---|---|
| F# | [mg/mL] | [10 mM] | pH | [mM] | [mM] |
| 3-F1 | 25/50 | L-His/L-His HCl | 5 | — | — |
| 3-F2 | 25/50 | L-His/L-His HCl | 5 | L-Lysine HCl 100 mM | — |
| 3-F3 | 25/50 | L-His/L-His HCl | 5 | L-Arginine HCl 200 mM | — |
| 3-F4 | 25/50 | L-His/L-His HCl | 5 | Glycine 150 mM | — |
| 3-F5 | 25/50 | L-His/L-His HCl | 5 | Glycinamide HCl 100 mM | — |
| 3-F6 | 25/50 | L-His/L-His HCl | 5 | Proline 100 mM | — |
| 3-F7 | 25/50 | L-His/L-His HCl | 5 | — | MgCl₂ 200 mM |
| 3-F8 | 25/50 | L-His/L-His HCl | 5 | — | NaCl 250 mM |

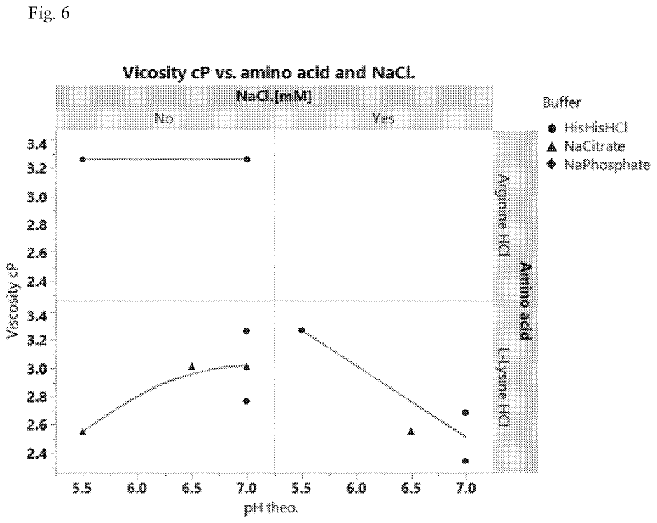

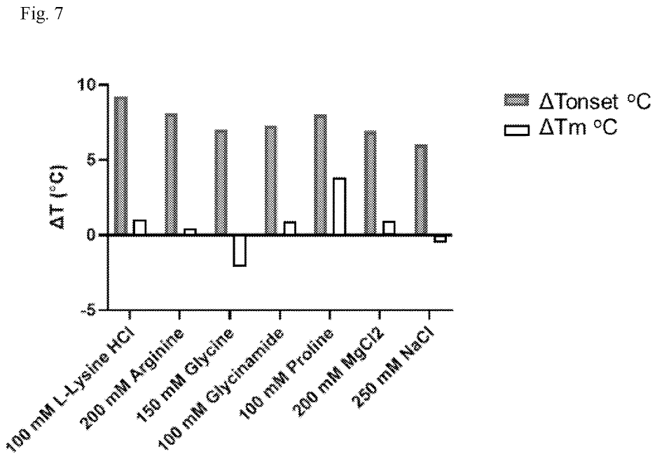

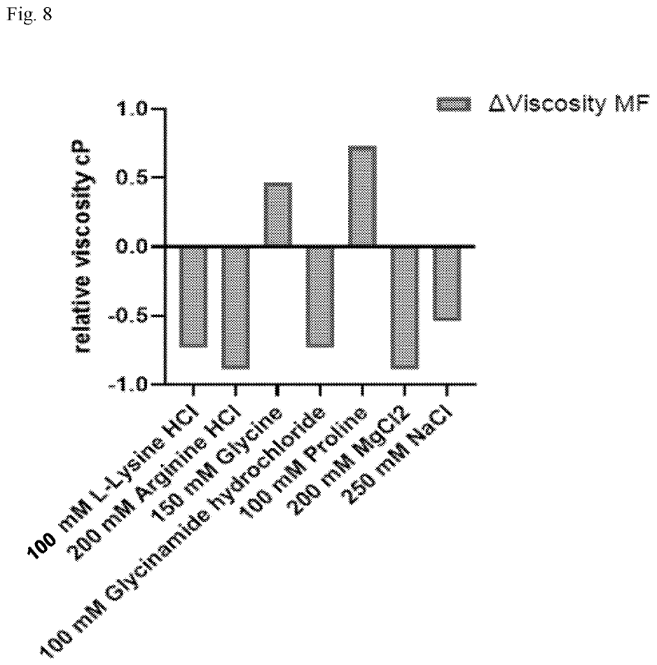

Changing of melting temperature in each formulation (ΔTₒₙₛₑₜ) showed that lysine offers the best performance in terms of thermal stability compared to other tested agents (FIG. 7). The most prominent effects on viscosity reduction were observed in formulations comprising MgCl₂, arginine, or lysine (FIG. 8).

### Example 4. Impact of Various Excipients on Colloidal and Conformational (Thermal) Stability

In Example 4, the effect of different types of surfactants, buffer types, and stabilizers on conformational (thermal) and colloidal stability of formulations comprising the bispecific antibody of Examples 1 and 2 was evaluated utilizing the same procedures. Stability was assessed at different pH values. Lysine and/or NaCl were included as viscosity reducing agents, and methionine was added as an anti-oxidant agent. The design and evaluation of the formulation conditions was done using JMP (SAS Institute Inc., Cary, NC, 1989-2023). A total of 18 different formulations (4-F1 to 4-F18) were tested.

TABLE 5
Formulations 4-F1 to 4-F18

| | | Buffer | | L-Lysine | | | | |
|---|---|---|---|---|---|---|---|---|
| | mAb | type | | HCl | NaCl | Sugar | L-Met | Surfactant |
| F# | [mg/mL] | [20 mM] | pH | [mM] | [mM] | [mM] | [mM] | [%] |
| 4-F1 | 10 | L-His/L-His | 7 | — | 100 | 220 mM | — | — |
| | | HCl | | | | trehalose | | |
| | | | | | | dihydrate | | |
| 4-F2 | 10 | L-His/L-His | 7 | — | 100 | 220 mM | — | 0.04% |
| | | HCl | | | | trehalose | | PS20 |
| | | | | | | dihydrate | | |
| 4-F3 | 10 | L-His/L-His | 7 | — | 100 | 220 mM | — | 0.04% |
| | | HCl | | | | trehalose | | PS80 |
| | | | | | | dihydrate | | |
| 4-F4 | 10 | L-His/L-His | 7 | — | 100 | 220 mM | — | 0.1% |
| | | HCl | | | | trehalose | | Polox188 |
| | | | | | | dihydrate | | |
| 4-F5 | 10 | L-His/L-His | 7 | 150 | — | 220 mM | — | — |
| | | HCl | | | | trehalose | | |
| | | | | | | dihydrate | | |
| 4-F6 | 10 | L-His/L-His | 7 | 150 | — | 220 mM | — | 0.04% |
| | | HCl | | | | trehalose | | PS20 |
| | | | | | | dihydrate | | |
| 4-F7 | 10 | L-His/L-His | 7 | 150 | — | 220 mM | — | 0.04% |
| | | HCl | | | | trehalose | | PS80 |
| | | | | | | dihydrate | | |
| 4-F8 | 10 | L-His/L-His | 7 | 150 | — | 220 mM | — | 0.1% |
| | | HCl | | | | trehalose | | Polox188 |
| | | | | | | dihydrate | | |
| 4-F9 | 10 | L-His/L-His | 7 | 100 | 100 | 220 mM | — | 0.04% |
| | | HCl | | | | trehalose | | PS80 |
| | | | | | | dihydrate | | |
| 4-F10 | 10 | L-His/L-His | 7 | 150 | — | 220 mM | 10 | 0.04% |
| | | HCl | | | | trehalose | | PS80 |
| | | | | | | dihydrate | | |
| 4-F11 | 10 | L-His/L-His | 5.5 | 150 | — | 220 mM | 10 | 0.04% |
| | | HCl | | | | trehalose | | PS80 |
| | | | | | | dihydrate | | |
| 4-F12 | 10 | L-His/L-His | 5.5 | 150 | — | 220 mM | — | 0.04% |
| | | HCl | | | | trehalose | | PS80 |
| | | | | | | dihydrate | | |
| 4-F13 | 10 | L-His/L-His | 5.5 | 150 | — | 220 mM | — | 0.04% |
| | | HCl | | | | trehalose | | PS20 |
| | | | | | | dihydrate | | |
| 4-F14 | 10 | Na-Citrate | 5.5 | 150 | — | 220 mM | — | 0.04% |
| | | | | | | trehalose | | PS80 |
| | | | | | | dihydrate | | |
| 4-F15 | 10 | Na-Citrate | 5.5 | 100 | 100 | 220 mM | — | 0.04% |
| | | | | | | trehalose | | PS80 |
| | | | | | | dihydrate | | |
| 4-F16 | 10 | L-His/L-His | 7 | 150 | — | 120 mM | — | 0.04% |
| | | HCl | | | | Sucrose | | PS20 |
| 4-F17 | 10 | L-His/L-His | 7 | 150 | — | 120 mM | — | 0.04% |
| | | HCl | | | | Sucrose | | PS80 |
| 4-F18 | 10 | L-His/L-His | 7 | 150 | — | 120 mM | — | 0.1% |
| | | HCl | | | | Sucrose | | Polox 188 |

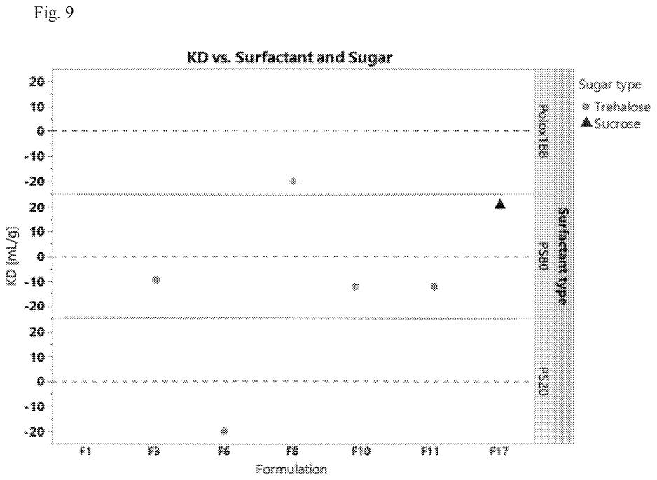

Formulations containing PS80 showed improved colloidal stability versus PS20-containing formulations (FIG. 9). Further, in terms of thermal stability, PS80 performed better than PS20, with Polox188 also showing good performance. Formulations with higher pH in L-His/L-His HCl buffer resulted in overall improved thermal stability (FIG. 10 and FIG. 11).

### Example 5. Viscosity and Long-Term Stability of Formulations Comprising High Concentrations of Bispecific Antibody

In Example 5, different formulations comprising 100-150 mg/mL of the anti-IL-18/IL-13 bispecific antibody (anti-IL-18 sequence: SEQ ID NO: 41 (VH1), SEQ ID NO: 13 (VL1), SEQ ID NO: 42 (HCl), SEQ ID NO: 14 (LC1); anti-IL-13 sequence: SEQ ID NO: 55 (VH2), SEQ ID NO: 27 (VL2), SEQ ID NO: 56 (HC2), SEQ ID NO: 28 (LC2)) were assessed for liquid stability under long-term storage and/or stressed conditions. A total of 10 different formulations (5-F1 to 5-F10) were tested, including formulations comprising different buffer types, pH values, amino acids, sugar concentrations, and surfactant concentrations.

TABLE 6
Formulations 5-F1 to 5-F10

| | | | | | L-Lysine | | | | |
|---|---|---|---|---|---|---|---|---|---|
| | mAb | Buffer | Buffer | | HCl | NaCl | Trehalose | L-Methionine | PS80 |
| F# | [mg/mL] | type | [mM] | pH | [mM] | [mM] | [mM] | [mM] | [%] |
| 5-F1 | 150 | Na- | 20 | 6.5 | 100 | 100 | 120 | 5 | 0.04 |
| | | Citrate | | | | | | | |
| 5-F2 | 150 | L-His/L- | 20 | 6.5 | 0 | 0 | 0 | 0 | 0 |
| | | His HCl | | | | | | | |
| 5-F3 | 150 | L-His/L- | 20 | 6.5 | 150 | 0 | 120 | 5 | 0.04 |
| | | His HCl | | | | | | | |
| 5-F4 | 150 | L-His/L- | 20 | 6.5 | 0 (150 | 0 | 120 | 5 | 0.04 |
| | | His HCl | | | mM L- | | | | |
| | | | | | arginine | | | | |
| | | | | | HCl) | | | | |
| 5-F5 | 150 | L-His/L- | 20 | 6.5 | 150 | 0 | 120 | 0 | 0.04 |
| | | His HCl | | | | | | | |
| 5-F6 | 150 | L-His/L- | 20 | 6.5 | 150 | 0 | 50 | 5 | 0.04 |
| | | His HCl | | | | | | | |
| 5-F7 | 150 | L-His/L- | 20 | 6.5 | 150 | 0 | 220 | 5 | 0.04 |
| | | His HCl | | | | | | | |
| 5-F8 | 150 | L-His/L- | 20 | 6 | 150 | 0 | 120 | 5 | 0.04 |
| | | His HCl | | | | | | | |
| 5-F9 | 150 | L-His/L- | 20 | 7 | 150 | 0 | 120 | 5 | 0.04 |
| | | His HCl | | | | | | | |
| 5-F10 | 100 | L-His/L- | 20 | 6.5 | 150 | 0 | 120 | 5 | 0.04 |
| | | His HCl | | | | | | | |

The compounded formulations were sterile-filtered through a 0.22 μm PVDF filter and aliquoted in 6R vials each filled with 1.2 mL solution, stoppered and crimped. Formulations were incubated in stability chambers at 5° C., 25° C. and 40° C. and analyzed according to the Table below.

Specifically, study conditions and sampling points (X) were as follows:

TABLE 7
Study conditions and sampling points

| Study | | | | | | |
|---|---|---|---|---|---|---|
| condition | Storage condition | T0 | 2 W | 4 W | 3 M | 6 M |
| Stability | 5° C. | | — | | X | X |
| samples | 25° C. | X | X | X | X | — |
| | 40° C. | | X | X | — | — |
| Freeze thaw | Samples were | X | — | — | — | — |
| stress | transferred at room | | | | | |
| (5 cycles) | temperature until | | | | | |
| | visually thawed | | | | | |
| | before analysis | | | | | |
| Shaking | Shaking for 48 h at | X | — | — | — | — |
| stress | room temperature | | | | | |
| samples | and stored at 2-8° C. | | | | | |
| | before analysis | | | | | |
| Forced | 40° C. | X | — | X | — | — |
| oxidation | | | | | | |
| samples | | | | | | |

Mechanical stress in 2 different categories (shaking and freeze thaw stress) and forced oxidation was applied to tested formulations. Shaking stress was applied using a Heidolph MultiReax vibrational shaker at room temperature for 48 h (1000 rpm/level 5). Afterwards, the stressed vials were stored at 2-8° C. before being analyzed.

Five freeze and thaw cycles were applied by placing a vial into a <−60° C. (−80° C.) freezer for at least 16 h, followed by positioning on a lab bench at room temperature until visually thawed. Then, the vial was placed back into the freezer. This process was repeated 5 times. The last thawing step was applied before sample analysis.

Forced oxidation sensitivity was assessed in real time by 10 mM hydrogen peroxide spiking to assess the impact of VHP (vaporized hydrogen peroxide) on the formulation oxidation (formulations were diluted with placebo up to 10 mg/mL) at TO and over stress storage conditions (40° C. for 4 weeks).

### Results

### Osmolality

Formulation osmolality was measured using an Advanced Instruments Model 2020 osmometer (Advanced Instruments, MA, USA), which utilizes freezing point depression (FPD) for measurements. All measurements were performed according to the manufacturer's instructions. As shown in FIG. 12, osmolality was within <600 mOsm/kg for all formulations but 5-F1 (citrate buffer) and 5-F7 (histidine buffer, 220 mM trehalose, pH 7).

### Viscosity

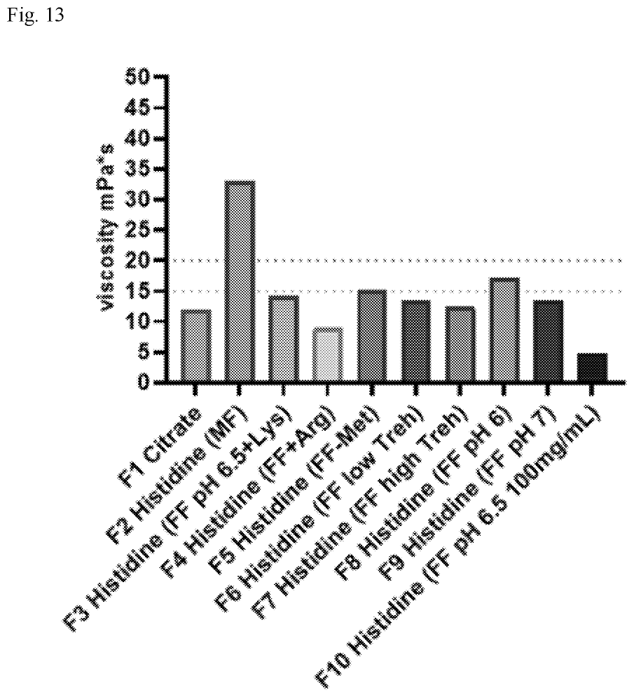

Formulation viscosity was measured as described in Example 2. The unformulated anti-IL-18/IL-13 bispecific antibody at a 150 mg/mL concentration has a viscosity of ≥25 cP. As shown in FIG. 13, all formulations but 5-F2 (minimally formulated) showed a viscosity of <20 cP. Lysine and arginine were effective in decreasing formulation viscosity.

### Thermal Stability

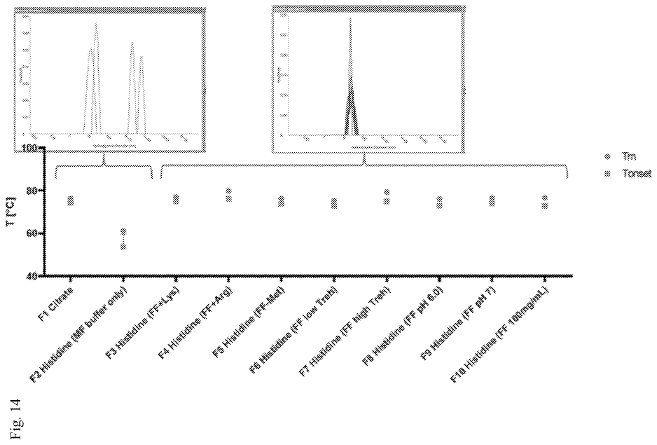

Thermal stability was measured as described in Examples 1 and 2. All formulations showed high and comparable conformational (thermal) stability, with an increase in both Tₒₙₛₑₜ & Tₘ of up to 15-20° C. However, dynamic light scattering results showed two different populations of a different protein size at TO for 5-F1 (citrate buffer) and 5-F2 (minimally formulated) (FIG. 14).

### Turbidity

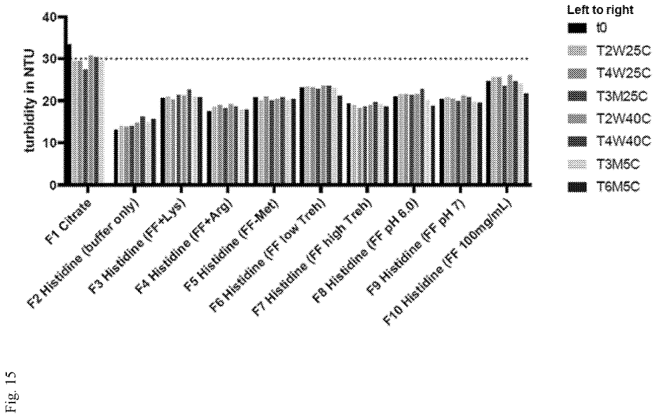

Turbidity values for formulations 5-F2 to 5-F10 (measured using a 2100 AN Turbidimeter (Hach, IA, USA)) were below 30 NTU, and thermal stress did not have a notable impact (25° C. for up to 3 months and 40° C. for up to 4 weeks). 5-F1 (in citrate buffer) showed a higher turbidity value compared to the other formulations (FIG. 15).

Purity Measured with Size Exclusion Chromatography (SEC)

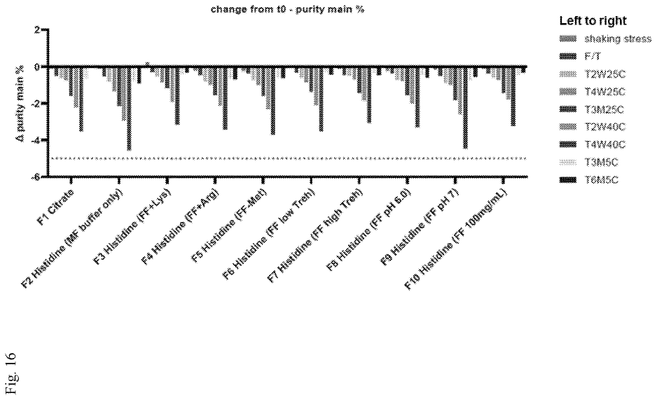

No relevant decrease in main peak purity was observed for all the formulations after 6 months storage at 5° C. and after 3 months at 25° C. compared to the TO time point. Stress conditions (40° C. for up to 4 weeks storage) resulted in a decrease in monomer content for all the formulations, with 5-F3 and 5-F7 performing the best (FIG. 16). This decrease was highest for 5-F2 (minimally formulated) and 5-F9 (histidine buffer, pH 7). Shaking and freeze thaw stress did not impact SEC monomer content.

Sum of Aggregates and Fragments Measured with Size Exclusion Chromatography (SEC)

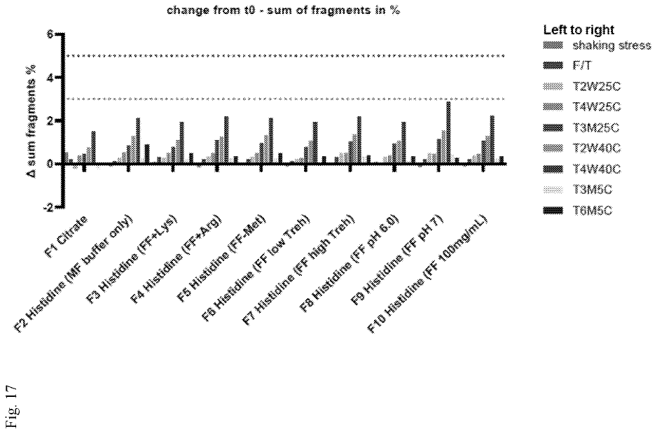

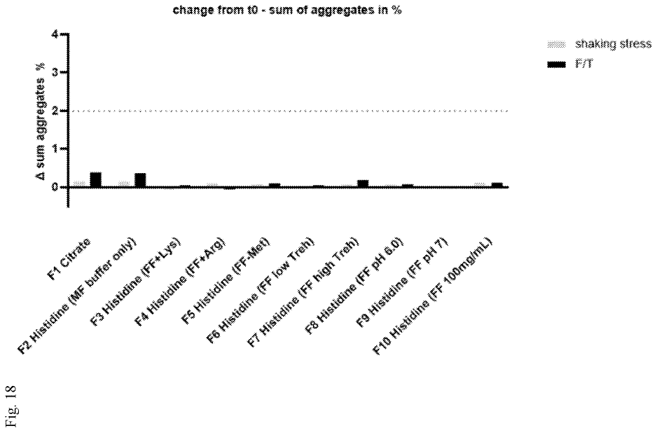

No significant change in sum of fragments and aggregates by SEC was observed for the formulations stored at 5° C. for 6 months and at 25° C. for 3 months compared to the TO time point (FIG. 17, FIG. 18). At 40° C. (up to 4 weeks storage), there was an increase in sum of fragments and aggregates for all the formulations, with 5-F3, 5-F6 and 5-F8 showing the smallest increase (FIG. 17, FIG. 18). 5-F2 showed the highest number of aggregates (FIG. 18), whereas 5-F7 showed the highest number of fragments species at 40° C. stability storage (FIG. 17).

Basic and Acidic Variants Measured with Capillary Zone Electrophoresis (CZE)

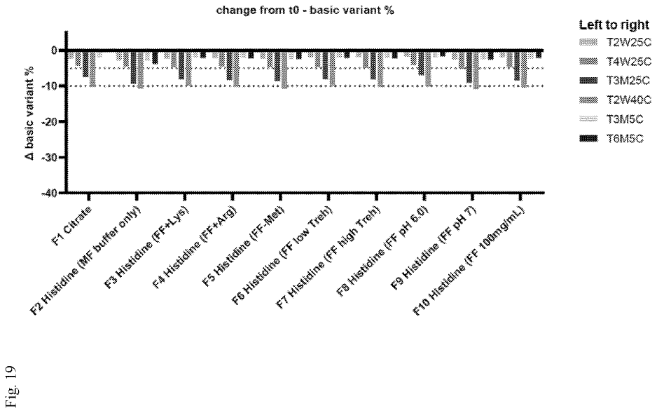

There were no notable differences in CZE basic variants between the formulations after 6 months storage at 5° C. A slight change in the number of basic variants under stress conditions (25° C. up to 3 months and 40° C. up to 2 weeks) was observed for all formulations (FIG. 19).

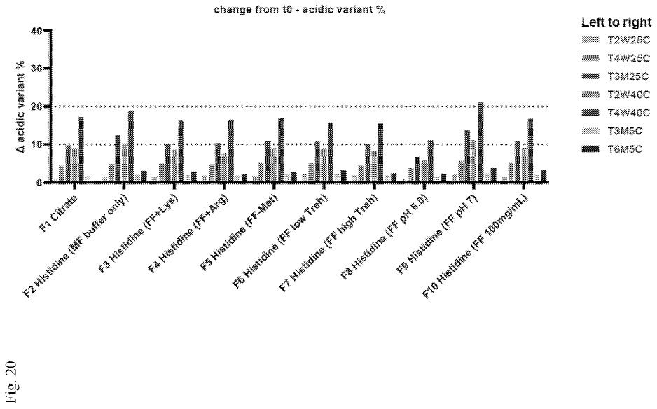

No relevant differences in acidic species were observed between the formulations after 6 months storage at 5° C. With thermal stress, an impact of higher pH towards the increase of sum of acidic peaks was observed (FIG. 20). 5-F7 showed the highest number of acidic variants at 40° C. (4 weeks).

Post-Translational Modifications Measured with Multi-Attribute Method Mass Spectrometry (MAM-MS)

Deamidation, isomerization, and pyroglutamate formation was assessed with MAM-MS using an LC-ESI-MS system in formulation samples stored at 40° C. (4 weeks) relative to the TO timepoint (formulation average).

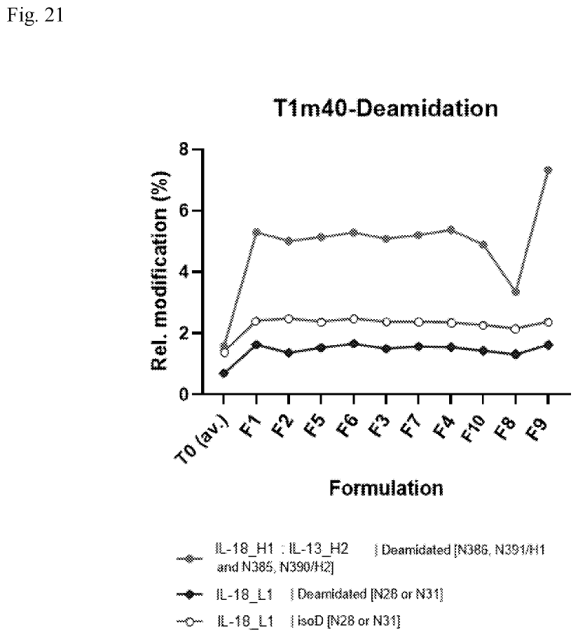

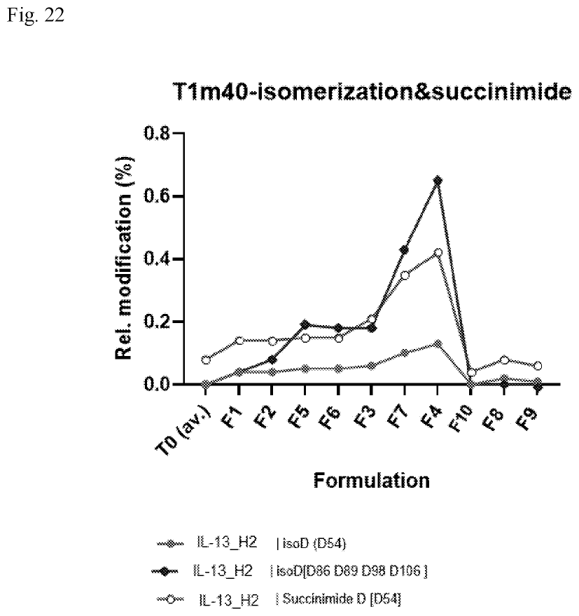

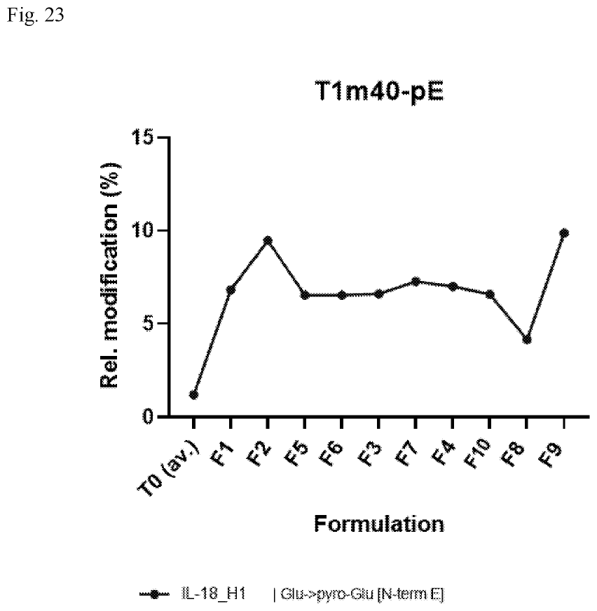

FIG. 21 shows the impact of temperature stress on deamidation. A lower deamidation level of amino acid residues N386, N391 (HCl; SEQ ID NO: 42)/N385, N390 (HC2; SEQ ID NO: 56) in formulation 5-F8 (pH 6) was observed. FIG. 22 shows the impact of temperature stress on isomerization. 5-F4 (arginine) and 5-F7 (higher trehalose conc.) showed higher isoD formation (amino acid residues D54, D86, D89, D98, D106; SEQ ID NO: 56) and succinimide (amino acid residue D54; SEQ ID NO: 56) compared to the other formulations. FIG. 23 shows the impact of temperature stress on pyroglutamate formation (N-terminal E residue; SEQ ID NO: 42). A higher impact was observed in formulation 5-F2 (minimally formulated) and lower level in 5-F8 (pH 6).

Forced Oxidation with H₂O₂

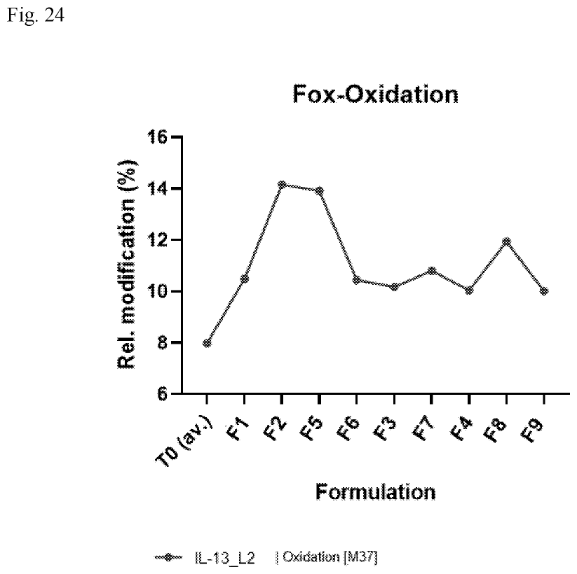

The impact of hydrogen peroxide treatment on formulations stored at 40° C. (4 weeks) relative to the TO timepoint (formulation average) was assessed with MAM-MS. FIG. 24 shows that the largest impact (oxidation of M37 residue; SEQ ID NO: 28) was observed in formulations wherein methionine was absent.

### Potency

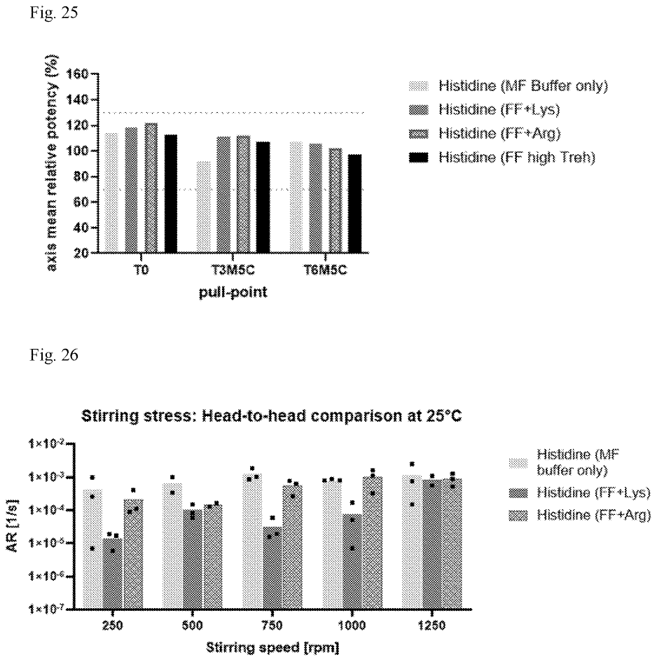

Potency change over time was measured (using ELISA; binding to IL-13, binding to IL-18) in non-stressed samples stored at 5° C. for 3 and 6 months for formulations 5-F2 (minimally formulated), 5-F3 (lysine), 5-F4 (arginine), and 5-F7 (high trehalose). Potency was maintained in tested formulations (FIG. 25).

### Aggregation Rate Following Shear Stress

The impact of shear stress on aggregation rate was evaluated for formulations 5-F2 (minimally formulated), 5-F3 (lysine) and 5-F4 (arginine). Aggregation rate (AR) was measured utilizing an ARGEN (Aggregate Rate GENerator) instrument (Yokogawa Fluence Analytics, TX, USA). ARGEN functions by analyzing variations in molecular size (Mw (t)) over time (dt) under thermal and/or mechanical stress (Mo in the following formula refers to initial molecular weight):

$$AR ( {S}^{- 1} ) = \frac{d ( \frac{Mw ( t )}{Mo} )}{dt}$$

The detection method leverages the well-established technique of right angle static light scattering (SLS) (e.g., see Drenski et al. (2013), Anal Biochem 437 (2): 185-197). Measurements were done according to the manufacturer's instructions, by applying 5 different stirring speed rpm values (250, 500, 750, 1000, and 1250 rpm) at 25° C. FIG. 26 demonstrates that lysine provides significantly better protection against shear stress than the one provided by arginine, and has an improved stabilizing effect in addition to its viscosity reducing properties.

1. A stable liquid pharmaceutical composition comprising a bispecific antibody, wherein the bispecific antibody comprises:
i) a first part that binds to IL-18 comprising a first heavy chain (HCl) comprising the amino acid sequence set forth in SEQ ID NO: 42 and a first light chain (LC1) comprising the amino acid sequence set forth in SEQ ID NO: 14, and;
ii) a second part that binds to IL-13 comprising: a second heavy chain (HC2) comprising the amino acid sequence set forth in SEQ ID NO: 56 and a second light chain (LC2) comprising the amino acid sequence set forth in SEQ ID NO: 28,
wherein the pharmaceutical composition comprises from about 15 mg/mL to about 200 mg/ml of the bispecific antibody and from about 100 mM to about 250 mM L-lysine or a pharmaceutically acceptable salt thereof, and wherein the pharmaceutical composition has a pH of from about 6 to about 7.

2. (canceled)

3. (canceled)

4. The pharmaceutical composition according to claim 1, wherein the pharmaceutical composition has a viscosity of about ≤20 cP, measured at a temperature of about 25° C.

5. (canceled)

6. The pharmaceutical composition according to claim 1, wherein the pharmaceutical composition comprises from about 100 mM to about 175 mM L-lysine or a pharmaceutically acceptable salt thereof.

7. The pharmaceutical composition according to claim 1, wherein the pharmaceutical composition comprises about 150 mM L-lysine or a pharmaceutically acceptable salt thereof).

8. (canceled)

9. (canceled)

10. The pharmaceutical composition according to claim 1, wherein the pharmaceutical composition has a pH of about 6.5.

11. The pharmaceutical composition according to claim 1, wherein the pharmaceutical composition comprises from about 50 mg/mL to about 175 mg/mL, from about 75 mg/mL to about 175 mg/mL, or from about 100 mg/mL to about 175 mg/ml of the bispecific antibody.

12. The pharmaceutical composition according to claim 1, wherein the pharmaceutical composition comprises about 150 mg/mL of the bispecific antibody.

13. The pharmaceutical composition according to claim 1, wherein the pharmaceutical composition comprises about 2.5 mM or more L-methionine or a pharmaceutically acceptable salt thereof.

14. The pharmaceutical composition according to claim 1, wherein the pharmaceutical composition comprises about 5 mM L-methionine or a pharmaceutically acceptable salt thereof.

15. (canceled)

16. The pharmaceutical composition according to claim 1, wherein the pharmaceutical composition comprises a sugar stabilizer.

17. The pharmaceutical composition according to claim 16, wherein the sugar stabilizer is trehalose or sucrose.

18. The pharmaceutical composition according to claim 1, wherein the pharmaceutical composition comprises from about 50 mM to about 220 mM or from about 100 mM to about 150 mM trehalose or sucrose.

19. The pharmaceutical composition according to claim 18, wherein the pharmaceutical composition comprises about 120 mM trehalose or sucrose.

20. The pharmaceutical composition according to claim 1, wherein the pharmaceutical composition comprises a buffering agent.

21. The pharmaceutical composition according to claim 20, wherein the buffering agent is selected from the group consisting of an acetate buffer, a citrate buffer, a phosphate buffer, a Tris buffer, a histidine buffer, and a combination thereof.

22. The pharmaceutical composition according to claim 1, wherein the pharmaceutical composition comprises from about 5 mM to about 50 mM or from about 10 mM to about 25 mM of an L-histidine buffer.

23. The pharmaceutical composition according to claim 22, wherein the pharmaceutical composition comprises about 20 mM of the L-histidine buffer.

24. The pharmaceutical composition according to claim 1, wherein the pharmaceutical composition comprises a surfactant.

25. The pharmaceutical composition according to claim 24, wherein the surfactant is selected from the group consisting of polysorbate 80 (PS80), polysorbate 20 (PS20), Polox188, and a combination thereof.

26. The pharmaceutical composition according to claim 1, wherein the pharmaceutical composition comprises from about 0.01% (w/v) to about 0.1% (w/v) or from about 0.02% (w/v) to 0.08% (w/V) polysorbate 80 (PS80) or polysorbate 20 (PS20).

27. The pharmaceutical composition according to claim 1, wherein the pharmaceutical composition comprises about 0.04% (w/v) polysorbate 80 (PS80).

28.-38. (canceled)

39. The pharmaceutical composition according to claim 13, wherein the pharmaceutical composition is stable at a temperature of about 5° C. for at least 6 months.

40. The pharmaceutical composition according to claim 1, wherein the pharmaceutical composition is stable at a temperature of about 25° C. for at least 3 months.

41. The pharmaceutical composition according to claim 1, wherein the pharmaceutical composition is stable at a temperature of about 40° C. for at least 4 weeks.

42. The pharmaceutical composition according to claim 1, wherein the pharmaceutical composition is suitable for subcutaneous administration.

43.-50. (canceled)

51. The A stable liquid pharmaceutical composition, wherein the pharmaceutical composition comprises:
about 150 mg/ml of a bispecific antibody comprising:
i) a first part that binds to IL-18 comprising a first heavy chain (HCl) comprising SEQ ID NO: 42 and a first light chain (LC1) comprising SEQ ID NO: 14, and;
ii) a second part that binds to IL-13 comprising a second heavy chain (HC2) comprising SEQ ID NO: 56 and a second light chain (LC2) comprising SEQ ID NO: 28;
about 20 mM of an L-histidine buffer;
about 120 mM trehalose;
about 150 mM L-lysine) or a pharmaceutically acceptable salt thereof;
about 5 mM L-methionine or a pharmaceutically acceptable salt thereof;
about 0.04% (w/v) polysorbate 80 (PS80),
and wherein the pharmaceutical composition has a pH of about 6.5.

52.-56. (canceled)

57. A method of preventing and/or treating an IL-18 and/or IL-13 related disease or disorder in a human subject in need thereof, comprising administering a therapeutically effective amount of the pharmaceutical composition of claim 1 to the subject.

58. The method according to claim 57, wherein the IL-18 and/or IL-13 related disease or disorder is an inflammatory and/or immune disease or disorder.

59. The method according to claim 57, wherein the IL-18 and/or IL-13 related disease or disorder is a skin disease or disorder.

60. The method according to claim 57, wherein the IL-18 and/or IL-13 related disease or disorder is atopic dermatitis, or a related condition.

61. (canceled)

62. The method according to claim 60, wherein the atopic dermatitis is moderate to severe atopic dermatitis.

63. A pharmaceutical product comprising a container comprising the pharmaceutical composition of claim 1.
